(19) United States
(12) Patent Application Publication
(10) Pub. No.: US 2025/0262287 A1
(43) Pub. Date: Aug. 21, 2025

(54) ARTHROSPIRA PLATENSIS ORAL VACCINE DELIVERY PLATFORM

(71) Applicant: Lumen Bioscience, Inc., Seattle, WA (US)

(72) Inventors: James ROBERTS, Seattle, WA (US); Michael A. TASCH, Seattle, WA (US); Tracy SAVERIA, Seattle, WA (US)

(21) Appl. No.: 19/202,734

(22) Filed: May 8, 2025

Related U.S. Application Data
(62) Division of application No. 17/056,306, filed on Nov. 17, 2020.
(60) Provisional application No. 62/672,891, filed on May 17, 2018.

(51) Int. Cl. A61K 39/015; A61K 39/00; A61P 37/04

(52) U.S. Cl. CPC A61K 39/015; A61K 39/00; A61P 37/04; A61K 2039/51; A61K 2039/542; A61K 2039/545; C12N 2730/10034; C12N 2750/14334

(57) ABSTRACT

The present disclosure provides oral antigenic compositions comprising a recombinant Spirulina comprising at least one exogenous antigenic epitope. Oral antigenic compositions of the present disclosure can be used as vaccines. Oral antigenic compositions of the present disclosure can be used to induce a protective immune response to infectious microorganism, tumor antigens, or self-antigens.

### CROSS REFERENCE TO RELATED APPLICATIONS

This Application is a divisional of U.S. application Ser. No. 17/056,306, filed Nov. 17, 2020, which is a U.S. National Stage of PCT/US2019/032998, filed May 17, 2019, which claims the benefit of priority to U.S. Provisional Application No. 62/672,891, filed on May 17, 2018, the contents of each of which are hereby incorporated by reference in their entireties.

### INCORPORATION BY REFERENCE OF THE SEQUENCE LISTING

The contents of the electronic sequence listing (LUBI_024_02US_SeqList_ST26.xml; Size: 95,086 bytes; and Date of Creation: May 7, 2025) are herein incorporated by reference in its entirety.

### FIELD

The disclosure is directed to oral antigenic compositions. In particular, the disclosure provides oral antigenic compositions comprising recombinant Spirulina, wherein the recombinant Spirulina comprises one or more exogenous antigenic epitopes.

### BACKGROUND

Vaccination is an efficient and cost-effective form of inducing immunity in an individual and a population, thereby saving lives and reducing morbidity and/or disability for billions of people. Despite the early success of the oral polio vaccine, most vaccines are delivered parenterally, and as such are associated with pain, non-compliance, biohazardous medical waste and strict requirements for expensive production, transport and storage logistics (the “cold chain”) and for trained technical personnel. Oral/mucosal vaccines eliminate or significantly reduce these drawbacks. Oral vaccines have been attempted in numerous plant species as well as eukaryotic algae, various yeasts and some bacteria. These platforms have suffered from technical and logistical challenges, including but not limited to poor vaccine protein expression, inherent toxicity, unpalatability, poor IgG induction or expensive extraction/purification procedures. Thus, there is an unmet need to provide a delivery platform for the large-scale and cost-effective production of oral vaccines. The present disclosure provides a new oral vaccine platform that eliminates or reduces some of these draw backs and serves as both, a production as well as a delivery platform, for oral vaccines. Specifically, the present disclosure provides Arthrospira platensis, commonly known as Spirulina, engineered to express high amounts of target antigens in a form that can be ingested orally without purification.

### SUMMARY OF THE INVENTION

Provided herein are oral antigenic compositions comprising a recombinant Spirulina, wherein the recombinant Spirulina comprises at least one exogenous antigenic epitope. In some embodiments, the at least one exogenous antigenic epitope is comprised in an exogenous antigen expressed by Spirulina.

In some embodiments, the exogenous antigen is a naturally-occurring antigen. For example, a recombinant Spirulina may express one or more exogenous antigens such as circumsporozoite proteins or TRAP proteins from Plasmodium that contain one or more antigenic epitopes.

In some embodiments, the exogenous antigen is a fusion protein. In some embodiments, the fusion protein comprises a viral protein. In some embodiments, the viral protein is a virus-like particle (VLP)-forming protein.

In some embodiments, the fusion protein comprises a scaffold protein.

In some embodiments, at least 2, at least 3, at least 4, or at least 5 copies of a nucleic acid sequence encoding the at least one exogenous antigenic epitope are present in the recombinant Spirulina.

In some embodiments, 2, 3, 4, 5, 6, 8, 10, 15, 20, 25, 30, 40, or 50 copies of a nucleic acid sequence encoding the at least one exogenous antigenic epitope are present in the recombinant Spirulina.

In some embodiments, at least 2, at least 3, at least 4, or at least 5 copies of the at least one exogenous antigenic epitope are present in a single molecule of the exogenous antigen expressed in the recombinant Spirulina.

In some embodiments, 2, 3, 4, 5, 6, 8, 10, 15, 20, 25, 30, 40, or 50 copies of the at least one exogenous antigenic epitope are present in a single molecule of the exogenous antigen expressed in the recombinant Spirulina.

In some embodiments, within the molecule of the exogenous antigen, the copies of the exogenous antigenic epitope are linked in tandem.

In some embodiments, within the molecule of the exogenous antigen, the copies of the exogenous antigenic epitope are separated by a spacer sequence.

In some embodiments, within the molecule of exogenous antigen, some of the copies of the exogenous antigenic epitope are linked in tandem and the remaining copies of the exogenous antigenic epitope are separated by a spacer sequence.

In some embodiments, the spacer sequence is between about 1 and 50 amino acids long. In some embodiments, more than one spacer sequence is present within the molecule of the exogenous antigen.

In some embodiments, the recombinant Spirulina comprises at least 2, at least 3, at least 4, or at least 5 different antigenic epitopes.

In some embodiments, the at least one exogenous antigenic epitope present in a recombinant Spirulina is derived from an infectious microorganism, a tumor antigen or a self-antigen associated with an autoimmune disease.

In some embodiments, the infectious microorganism is a virus, bacterium, parasite, or fungus.

In some embodiments, the infectious microorganism is a bacterium selected from the group consisting of: Mycobacterium, Streptococcus, Staphylococcus, Shigella, Campylobacter, Salmonella, Clostridium, Corynebacterium, Pseudomonas, Neisseria, Listeria, Vibrio, Bordetella, Helicobacter pylori, and Legionella.

In some embodiments, the infectious microorganism is a virus selected from the group consisting of: bacteriophage, RNA bacteriophage (e.g. MS2, AP205, PP7 and Qβ), Infectious Haematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, Poliovirus, Norovirus, Zika Virus, Denge Virus, Rabies Virus, Newcastle Disease Virus, and White Spot Syndrome Virus.

In some embodiments, the infectious microorganism is a parasite selected from the group consisting of: Plasmodium, Trypanosoma, Toxoplasma, Giardia, Leishmania Cryptosporidium, helminthic parasites: Trichuris spp., Enterobius spp., Ascaris spp., Ancylostoma spp. and Necator spp., Strongyloides spp., Dracunculus spp., Onchocerca spp. and Wuchereria spp., Taenia spp., Echinococcus spp., and Diphyllobothrium spp., Fasciola spp., and Schistosoma spp.

In some embodiments, the infectious microorganism is a fungus selected from the group consisting of: Aspergillus, Candida, Blastomyces, Coccidioides, Cryptococcus, and Histoplasma.

In some embodiments, the infectious microorganism is a Plasmodium. In some embodiments, the Plasmodium is P. falciparum, P. malariae, P. ovale or P. vivax. In some embodiments, the Plasmodium is Plasmodium falciparum.

In some embodiments, the at least one exogenous antigenic epitope is from a Plasmodium antigen selected from the group consisting of: circumsporozoite protein, thrombospondin-related anonymous protein (TRAP), Apical Membrane Antigen 1 (AMA1), the major merozoite surface proteins 1-3 (MSP1-3), sexual stage antigen 25 (s25), and sexual stage antigen s230.

In some embodiments, the at least one exogenous antigenic epitope is from a circumsporozoite protein of a Plasmodium.

In some embodiments, the at least one exogenous antigenic epitope comprises the sequence of NANP.

In some embodiments, the recombinant Spirulina comprises at least 2 exogenous antigenic epitopes, wherein one of the exogenous antigenic epitope comprises the sequence of NANP and the second exogenous antigenic epitope comprises the sequence of NVDP.

In some embodiments, the recombinant Spirulina comprises at least 3 exogenous antigenic epitopes, wherein one of the exogenous antigenic epitope comprises the sequence of NANP, the second exogenous antigenic epitope comprises the sequence of NVDP, and the third exogenous antigenic epitope comprises the sequence of NPDP.

In some embodiments, the at least one exogenous antigenic epitope is from a glycoprotein (SEQ ID NO: 68) of IHNV.

In some embodiments, the at least one exogenous antigenic epitope is from a viral capsid protein of canine parvovirus. In some embodiments, the at least one exogenous antigenic epitope is from a viral capsid protein of canine parvovirus having SEQ ID NO: 69.

In some embodiments, the at least one exogenous antigenic epitope is from the gp41 subunit of an envelope glycoprotein of HIV. In some embodiments, the at least one exogenous antigenic epitope is from the gp41 subunit of an envelope glycoprotein of HIV having SEQ ID NO: 70.

In some embodiments, the at least one exogenous antigenic epitope is comprised in a fusion protein comprising an amino acid sequence derived from a capsid protein of a virus.

In some embodiments, the capsid protein is Hepadnaviridae core antigen (HBcAg).

In some embodiments, the capsid protein is woodchuck hepadnaviridae core antigen (WHcAg).

In some embodiments, the fusion protein comprises an amino acid sequence derived from WHcAg and an at least one exogenous antigenic epitope from the circumsporozoite protein of Plasmodium. In some embodiments, the at least one exogenous antigenic epitope from the circumsporozoite (CSP) protein of Plasmodium is from the CSP sequence listed in Table 3.

In some embodiments, the fusion protein comprises an amino acid sequence derived from WHcAg and an at least one exogenous antigenic epitope from a glycoprotein of IHNV having SEQ ID NO: 68.

In some embodiments, the fusion protein comprises an amino acid sequence derived from WHcAg and an at least one exogenous antigenic epitope from a viral capsid protein of canine parvovirus. In some embodiments, the at least one exogenous antigenic epitope is from a viral capsid protein of canine parvovirus having SEQ ID NO: 69.

In some embodiments, the fusion protein comprises an amino acid sequence derived from WHcAg and an at least one exogenous antigenic epitope from the gp41 subunit of an envelope glycoprotein of HIV. In some embodiments, the at least one exogenous antigenic epitope is from the gp41 subunit of an envelope glycoprotein of HIV having SEQ ID NO: 70.

In some embodiments, the fusion protein comprises an amino acid sequence derived from WHcAg and the at least one exogenous antigenic epitope selected from the group consisting of: NANP, NVDP, NPDP, and a combination thereof.

In some embodiments, the fusion protein comprises an amino acid sequence derived from WHcAg and an at least one exogenous antigenic epitope selected from Table 1.

In some embodiments, the at least one exogenous antigenic epitope is comprised in a fusion protein comprising a scaffold protein.

In some embodiments, the at least one exogenous antigenic epitope is linked to a scaffold protein at the N-terminus or the C-terminus, or in the body of the scaffold protein.

In some embodiments, the scaffold protein is selected from the oligomerization domain of C4b-binding protein (C4BP), cholera toxin b subunit, or oligomerization domains of extracellular matrix proteins.

In some embodiments, in a fusion protein comprising the at least one exogenous antigenic epitope and the scaffold protein, the at least one exogenous antigenic epitope and the scaffold protein are separated by about 1 to about 50 amino acids.

In some embodiments, at least 2, at least 3, at least 4, or at least 5 copies of the at least one exogenous antigenic epitope are present in a fusion protein expressed by recombinant Spirulina.

In some embodiments, the fusion protein comprises 2-1000 copies of the at least one exogenous antigenic epitope.

In some embodiments, the copies of the at least one exogenous antigenic epitope present in a fusion protein are linked in tandem and/or separated by a spacer sequence of between about 1 to about 50 amino acids.

In some embodiments, in a fusion protein comprising the at least one exogenous antigenic epitope and the scaffold protein, the fusion protein comprises multiple copies of the at least one exogenous antigenic epitope, wherein the at least one exogenous antigenic epitope and the scaffold protein are arranged in any one of the following patterns: (E)n-(SP), (SP)-(E)n, (SP)-(E)n-(SP), (E)n₁-(SP)-(E)n₂, (SP)-(E)n₁-(SP)-(E)n₂, and (SP)-(E)n₁-(SP)-(E)n₂-(SP), wherein E is the at least one exogenous antigenic epitope, SP is the scaffold protein, n, n₁, and n₂ represent the number of copies of the at least one exogenous antigenic epitope.

In some embodiments, the recombinant Spirulina comprises a fusion protein comprising one or more antigenic epitopes selected from Table 1.

In some embodiments, the recombinant Spirulina comprises a fusion protein comprising a sequence selected from Table 2.

In some embodiments, the recombinant Spirulina comprises a fusion protein comprising one or more antigenic epitopes from the sequences listed in Table 3.

In some embodiments, the recombinant Spirulina is non-living.

In some embodiments, the recombinant Spirulina is dried, spray dried, freeze-dried, or lyophilized.

In some embodiments, the oral antigenic composition comprises a pharmaceutically acceptable excipient.

In some embodiments, provided herein are methods of inducing an immune response in a subject in need thereof, comprising administering to the subject an oral antigenic composition described herein.

In some embodiments, methods of the disclosure induce a humoral immune response.

In some embodiments, methods of the disclosure induce a cellular immune response.

In some embodiments, methods of the disclosure induce an innate immune response.

In some embodiments, provided herein are methods of reducing the severity of an infection in a subject in need thereof, comprising administering to the subject an oral antigenic composition described herein, wherein the composition comprises at least one exogenous antigenic epitope derived from a microorganism causing the infection.

In some embodiments, methods of the disclosure reduce the severity of a viral, bacterial, parasitic, or fungal infection in a subject in need thereof.

In some embodiments, methods of the disclosure reduce the severity of malaria in a subject in need thereof.

In some embodiments, methods of the disclosure reduce the severity of an infection selected from tetanus, diphtheria, pertussis, pneumonia, meningitis, campylobacteriosis, mumps, measles, rubella, polio, flu, hepatitis, chickenpox, malaria, toxoplasmosis, giardiasis, or leishmaniasis.

In some embodiments, methods of the disclosure reduce the severity of an infection caused by a bacterium selected from the group consisting of: Mycobacterium, Streptococcus, Staphylococcus, Shigella, Campylobacter, Salmonella, Clostridium, Corynebacterium, Pseudomonas, Neisseria, Listeria, Vibrio, Bordetella, Helicobacter pylori, and Legionella.

In some embodiments, methods of the disclosure reduce the severity of an infection caused by a virus selected from the group consisting of: bacteriophage, RNA bacteriophage (e.g. MS2, AP205, PP7 and Qβ), Infectious Haematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, and Poliovirus, Norovirus, Zika virus, Denge Virus, Rabies Virus, Newcastle Disease Virus, and White Spot Syndrome Virus.

In some embodiments, methods of the disclosure reduce the severity of an infection caused by a parasite selected from the group consisting of: Plasmodium, Trypanosoma, Toxoplasma, Giardia, Leishmania, ,Cryptosporidium, helminthic parasites: Trichuris spp., Enterobius spp., Ascaris spp., Ancylostoma spp. and Necator spp., Strongyloides spp., Dracunculus spp., Onchocerca spp. and Wuchereria spp., Taenia spp., Echinococcus spp., and Diphyllobothrium spp., Fasciola spp., and Schistosoma spp.

In some embodiments, methods of reducing the severity of an infection in a subject in need thereof comprise administering a priming dose of an oral antigenic composition described herein and subsequently administering one or more booster doses of the oral antigenic composition.

In some embodiments, methods of reducing the severity of an infection in a subject in need thereof comprise administering a priming dose of an antigenic composition that is different from the oral antigenic composition and subsequently administering one or more booster doses of the oral antigenic composition.

In some embodiments, the booster dose is administered about two weeks, 1 month, 2 months, 3 months, 4 months, 6 months, 1 year, 2 years, and/or 5 years after the priming dose.

In some embodiments, provided herein are methods of making the oral antigenic composition described herein, the method comprising introducing a nucleic acid sequence encoding the at least one exogenous antigenic epitope into a Spirulina.

In some embodiments, provided herein are oral antigenic compositions comprising a recombinant Spirulina, wherein the recombinant Spirulina comprises at least one exogenous antigenic epitope, wherein a nucleic acid sequence encoding the at least one exogenous antigenic epitope is integrated into the Spirulina via homologous recombination.

In some embodiments, provided herein are oral antigenic compositions prepared by a method comprising: introducing a nucleic acid sequence encoding at least one exogenous antigenic epitope into a Spirulina and integrating the nucleic acid sequence into the Spirulina via homologous recombination.

### BRIEF DESCRIPTION OF THE DRAWINGS

FIG. 1A shows a schematic of the fusion protein described in Example 1 comprising Woodchuck Hepatitis Virus Capsid protein (WHcAg) and Plasmodium yoelii circumsporozoite (CSP) protein B cell epitopes and CSP T cell epitopes.

FIG. 1B shows a homodimer of WHcAg assembled into a virus-like particle (VLP).

FIG. IC shows a ribbon diagram of a WHcAg homodimer showing the spike (arrows) formed by the Major Insertion Region (MIR).

FIG. 1D shows a sonicated recombinant Spirulina culture comprising the fusion protein described in Example 1 before discontinuous sucrose density ultracentrifugation.

FIG. 1E shows a sonicated recombinant Spirulina culture comprising the fusion protein described in Example 1 after discontinuous sucrose density ultracentrifugation.

FIG. 1F shows the bottom fractions collected after discontinuous sucrose density ultracentrifugation of a sonicated recombinant Spirulina culture and resolved by native polyacrylamide gel electrophoresis (PAGE) or SDS-PAGE.

FIG. 1G shows a growth curve for a recombinant Spirulina culture described in Example 1.

FIG. 1H shows a scale-up to small pilot scale (100 liters) using Fence-type bioreactor with full spectrum LED lighting, illuminated glass tubing. O₂ scrubbing and CO₂ injection.

FIG. 1I shows an amino acid sequence of the fusion protein shown in FIG. 1A and the corresponding nucleotide sequence.

FIG. 2 shows a schematic of the experimental design (panel A); a graph summarizing the results of a CSP-ELISA assay (panel B); and a graph summarizing the results for a Day 5 blood smear data showing mean parasites per high powered field (panel C).

FIG. 3 shows a schematic of the experimental design (panel A); a graph summarizing results of liver burden as assessed by Plasmodium 18S IRNA RT-PCR (panel B); a graph summarizing results of an in vitro inhibition of spz invasion (ISI) assay; and a graph summarizing results of a CSP-ELISA assay (panel D).

FIG. 4A shows a schematic of the fusion protein comprising WHcAg domains and E1E2 epitopes from infectious haematopoietic necrosis virus (IHNV).

FIG. 4B shows an amino acid sequence of the fusion protein shown in FIG. 4A and the corresponding nucleotide sequence.

FIG. 4C shows a schematic of the fusion protein comprising WHcAg domains and DIII epitopes from IHNV.

FIG. 5A shows a schematic of the fusion protein comprising WHcAg and 2L21 B cell epitopes from canine parvovirus.

FIG. 5B shows an amino acid sequence of the fusion protein shown in FIG. 5A and the corresponding nucleotide sequence.

FIG. 6A shows a schematic of the fusion protein comprising WHcAg and 3L17 epitopes from canine parvovirus.

FIG. 6B shows an amino acid sequence of the fusion protein shown in FIG. 6A and the corresponding nucleotide sequence.

FIG. 7 shows a graph summarizing the results of a systemic IgG response to oral immunization of mice with recombinant Spirulina comprising the fusion proteins shown in FIGS. 5A and 6A.

FIG. 8A shows a schematic of the fusion protein comprising WHcAg domains and CSP B cell epitopes from Plasmodium falciparum.

FIG. 8B shows an amino acid sequence of the fusion protein shown in FIG. 8A and the corresponding nucleotide sequence.

FIG. 9. shows murine survival after immunization with P. falciparum CSP Spirulina vaccine and subsequent sporozoite challenge.

FIG. 10 shows a graph summarizing the results of an IgG response in mice, orally-dosed with Spirulina containing WHcAg nanoparticles with P. falciparum (NANPx) epitopes (PfCSP-VLP) and control mice orally dosed with Spirulina containing WHcAg nanoparticles without P. falciparum epitopes (empty VLP).

FIG. 11 shows a ribbon diagram of a human HepB core trimer of dimers.

FIG. 12 shows a ribbon diagram showing a “canyon” from C-term to spike.

FIG. 13 shows a ribbon diagram showing a “canyon” exit.

FIG. 14 shows a ribbon diagram of GCN4-pII coiled-coil trimerization domain.

FIG. 15 shows a ribbon diagram of GCN4-pII coiled-coil trimerization domain with mutations from HIV gp41.

FIG. 16 shows a ribbon diagram of N-terminal of HIV gp41-derived.

FIG. 17 shows a ribbon diagram of Juxtaposing GCN4-pII trimerization coiled-coil domain onto the Hepatitis B core protein VLP.

### DETAILED DESCRIPTION

Oral vaccines are safe, easy to administer and convenient for all ages. Various recombinant or attenuated viral or bacterial strains have been developed as carriers for oral delivery of vaccines. For example, both Salmonella typhimurium and Salmonella enterica have been engineered to express Plasmodium antigens or antigenic epitopes for oral vaccination (Schorr, J., et al., Surface expression of malarial antigens in Salmonella typhimurium: induction of serum antibody response upon oral vaccination of mice. Vaccine, 1991. 9(9): p. 675-81; Ruiz-Perez, F., et al., Expression of the Plasmodium falciparum immunodominant epitope (NANP)(4) on the surface of Salmonella enterica using the autotransporter MisL. Infect Immun, 2002. 70(7): p. 3611-20). While these studies induced protective antibodies and some T cell responses, they also required either extensive protein purification or use of live-attenuated organisms.

Another known vaccination strategy comprises the use of virus-like particles (VLPs), where antigenic targets are fused with VLPs. VLPs are non-infectious, robust and highly immunogenic nanoparticles that spontaneously form when viral capsid proteins are expressed in heterologous systems. Oral VLP delivery to healthy volunteers has been shown to be safe and effective. VLPs fused to malaria antigens have been expressed in plants (Jones, R. M., et al., A plant-produced Pfs25 VLP malaria vaccine candidate induces persistent transmission blocking antibodies against Plasmodium falciparum in immunized mice. PLOS One, 2013. 8(11): p. e79538). However, as with the Salmonella-based vaccines, these systems also require purification of the VLPs or use of live vectors for antigen delivery.

Chlamydomonas reinhardtii has been used to express blood-stage malarial proteins. When purified and admixed with recombinant heat labile toxin (LTB; homologous to cholera toxin B (CTB) in structure and function) and fed to mice, protective antibody responses were observed (Dauvillee, D., et al., Engineering the chloroplast targeted malarial vaccine antigens in Chlamydomonas starch granules. PLOS One, 2010. 5(12): p. e15424). In a different study, a Pfs25-CTB fusion was similarly expressed in C. reinhardtii and shown to be protective when purified and injected intraperitoneally (i.p.) (Gregory, J. A., et al., Algae-produced Pfs25 elicits antibodies that inhibit malaria transmission. PLoS One, 2012. 7(5): p. e37179). However when tested orally, non-purified Chlamydomonas/CTB-Pfs25 biomass induced Pfs25-specific non-protective IgA only, but not systemic IgG (Gregory, J. A., et al., Alga-produced cholera toxin-Pfs25 fusion proteins as oral vaccines. Appl Environ Microbiol, 2013. 79(13): p. 3917-25). In this study, whole recombinant biomass was tested orally; however, a protective immune response to the Pfs25 target antigen was not observed. The low expression (0.09% of total soluble protein) and/or degradation in the stomach were invoked as possible explanations for failure of whole recombinant biomass as a vaccine.

The present disclosure is the first in which Plasmodium antigens are expressed in edible prokaryotic algae and then administered to a subject, and where administration of the algae induces protective serum anti-parasite IgG antibodies. Furthermore, the expression levels of the exogenous antigens or the antigenic epitopes in the Spirulina delivery systems of the present disclosure are 10 to 100-fold higher compared to other systems.

Provided herein are oral antigenic compositions comprising a recombinant Spirulina comprising at least one exogenous antigenic epitope, methods of making, and use thereof.

Before describing certain embodiments in detail, it is to be understood that this disclosure is not limited to particular compositions or biological systems, which can vary. It is also to be understood that the terminology used herein is for the purpose of describing particular illustrative embodiments only, and is not intended to be limiting. The terms used in this specification generally have their ordinary meaning in the art, within the context of this disclosure and in the specific context where each term is used. Certain terms are discussed below or elsewhere in the specification, to provide additional guidance to the practitioner in describing the compositions and methods of the disclosure and how to make and use them. The scope and meaning of any use of a term will be apparent from the specific context in which the term is used. As such, the definitions set forth herein are intended to provide illustrative guidance in ascertaining particular embodiments of the disclosure, without limitation to particular compositions or biological systems.

Following long-standing patent law convention, the terms “a”, “an”, and “the” refer to “one or more” when used in this application, including the claims, unless clearly indicated otherwise. By way of example, “an antigenic epitope” means one epitope or more than one epitope.

As use herein, the term “antigenic composition” refers to a preparation which, when administered to a subject will induce a protective immune response that provides immunity to a disease or disorder, or can be used to treat a disease or disorder as described herein.

The term “antigen” as used herein refers to a protein or a peptide that binds to a receptor of an immune cell and induces an immune response in a human or an animal. The antigen can be from infectious microorganisms including viruses, bacteria, parasite, or fungi or the antigen can be a tumor antigen or a self-antigen associated with an autoimmune disease.

The term “antigenic epitope” as used herein refers to a short amino acid sequence, for example, of about 4 to 1000 amino acids, of an antigen that is recognized by, and binds to, a receptor of an immune cell and induces an immune response in a human or an animal. The antigenic epitopes of the present disclosure are from the antigens described above.

The term “subject” as used herein refers to a vertebrate or an invertebrate, and includes mammals, birds, fish, reptiles, and amphibians. Subjects include humans and other primates, including non-human primates such as chimpanzees and other apes and monkey species. Subjects include farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like; and aquatic animals such as fish, shrimp, and crustaceans.

### Oral Antigenic Compositions

Provided herein are oral antigenic compositions comprising a recombinant Spirulina, wherein the Spirulina is engineered to express at least one exogenous antigenic epitope.

In some embodiments, the at least one exogenous antigenic epitope is expressed in Spirulina by itself, i.e., the antigenic epitope is not fused to another protein.

In some embodiments, the at least one exogenous antigenic epitope expressed in Spirulina is comprised in an exogenous antigen. In some embodiments, the exogenous antigen is a natural antigen. For example, a recombinant Spirulina may express the entire circumsporozoite protein containing one or more antigenic epitopes or a portion or a domain of the circumsporozoite protein that contains one or more antigenic epitopes. In this case, the exogenous antigen is considered a natural antigen. Other examples of natural antigens that can be expressed in Spirulina to prepare oral antigenic compositions include hemagglutinin (HA), neuraminidase (NA), and matrix (M1) proteins of an influenza virus.

In addition to immunogenic epitopes, the present disclosure provides structures and/or ligands to stimulate the innate immune system (e.g. by engineering the epitopes into VLP structures). The innate immune system can be activated by adjuvant-like properties inherent in the VLP and/or adjuvants added to vaccine compositions. In some embodiments, these structures and/or ligands that stimulate the innate immune system include, but are not limited to, fragments of Salmonella flagellin, fliC, human and mouse TNF-alpha, and human and mouse CD40-Ligand.

In some embodiments, the exogenous antigen is a fusion protein. For example, in some embodiments, a recombinant Spirulina may express a fusion protein comprising at least one exogenous antigenic epitope and a portion of another protein such as a viral protein or a scaffold protein.

In some embodiments, at least one exogenous epitope is expressed in Spirulina as a fusion protein, wherein the fusion protein forms a three-dimensional structure (sometimes referred to herein as “particles”) that presents the at least one antigenic epitope in a spatially recognizable form that can elicit an innate and adaptive immune response. In some embodiments, the fusion protein that forms a three-dimensional structure may comprise multiple functional domains and one or more exogenous antigenic epitopes. Such fusion proteins can be engineered in a number of ways. In some embodiments, a fusion protein is a single polypeptide with multiple modular domains. An example of this is the woodchuck hepadnavirus core antigen (WHcAg) engineered with a B cell antigen at the Major Insertion Region/spike position, and a T cell epitope at the C-terminus. Another example is an RNA bacteriophage (ie, MS2, PP7, AP205 or Q_{β}), engineered to be a tandem dimer, with an antigen at the N-terminus, and a fragment of Salmonella flagellin at the C-terminus, thus combining an immunogenic epitope with an innate immune system stimulant to act as an intrinsic adjuvant, which self-organizes into a three-dimensional structure with two functional domains displayed on its surface. In some embodiments, recombinant Spirulina may express two heterologous polypeptides. For example, a recombinant Spirulina may express one gene that encodes a tandem RNA bacteriophage capsid protein dimer with an N-terminal antigenic structure, and a second gene that encodes an identical capsid dimer but with an adjuvant like Salmonella flagellin at its C-terminus. These two nearly identical polypeptides expressed in Spirulina can cooperatively form a three-dimensional mosaic particle in which the two polypeptides contribute to the “tiling” that forms a VLP capsid. Another example of this is to express a gene encoding a viral capsid protein like WHcAg or one of the RNA phage particles with an antigen genetically linked. and a second gene with the native viral protein. This allows for the avoidance of stearic conflicts that might arise if every particle had a bulky hybrid partner attached. The particles formed in this example can self-organize forming further higher-order structures.

In some embodiments, the recombinant Spirulina comprises a fusion protein comprising at least one exogenous antigenic epitope and a trimerization domain of certain proteins that naturally exist as trimers. Exemplary proteins that comprise trimerization domains are described below. For example, the HA protein from influenza virus (either the whole ectodomain or the minimal stem region) naturally forms trimers, and interfaces between monomeric subunits are considered to be important immunodominant epitopes. The fusion protein (F protein) from respiratory syncytial virus (RSV) is an obligate trimer. Similarly, Tumor Necrosis Factor alpha (TNFα) and the ligand for CD40 (CD40L) are obligate trimers. A recombinant Spirulina comprising a fusion protein comprising at least one exogenous antigenic epitope and a trimerization domain of any of these proteins is encompassed by the present disclosure. In exemplary embodiments, to facilitate trimerization, the inventors have genetically linked the WHcAg monomer to a number of coiled-coil domains that both facilitate trimer formation and situate bulky domains like influenza HA away from the potential stearic interference by the spike domains of the WHcAg. The inevtnors have used a trimerization derivative of the Saccharomyces cerevisiae transcription factor GCN4, a parallel trimeric-coiled coil, and a related structure based on CGN4 with the addition of mutations informed by the HIV GP41 trimer structure. The inventors have genetically linked these two trimers, with varying length linker sequences, to WHcAg, as well as to a number of RNA bacteriophages.

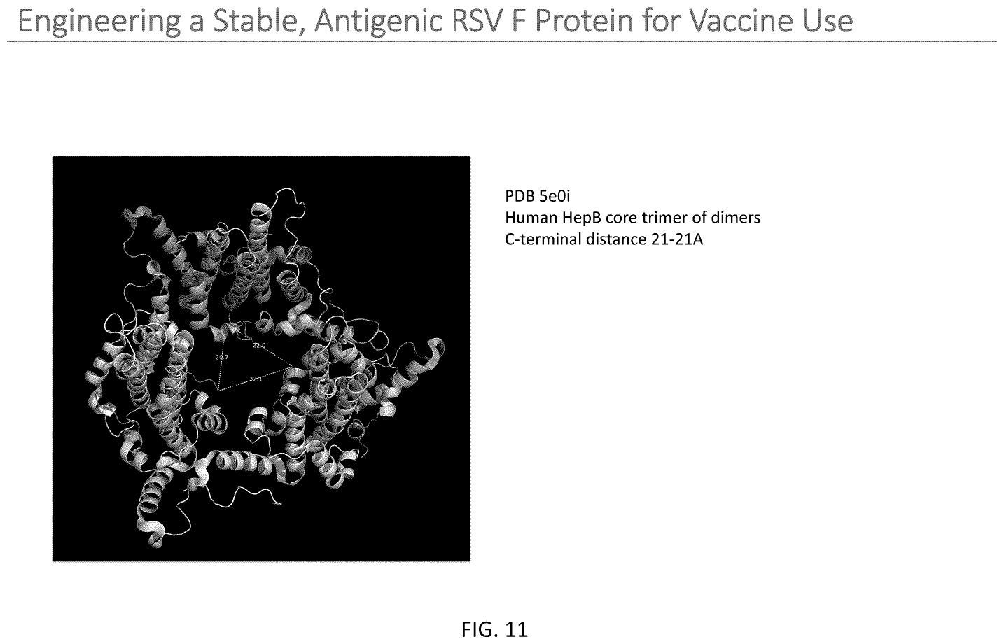

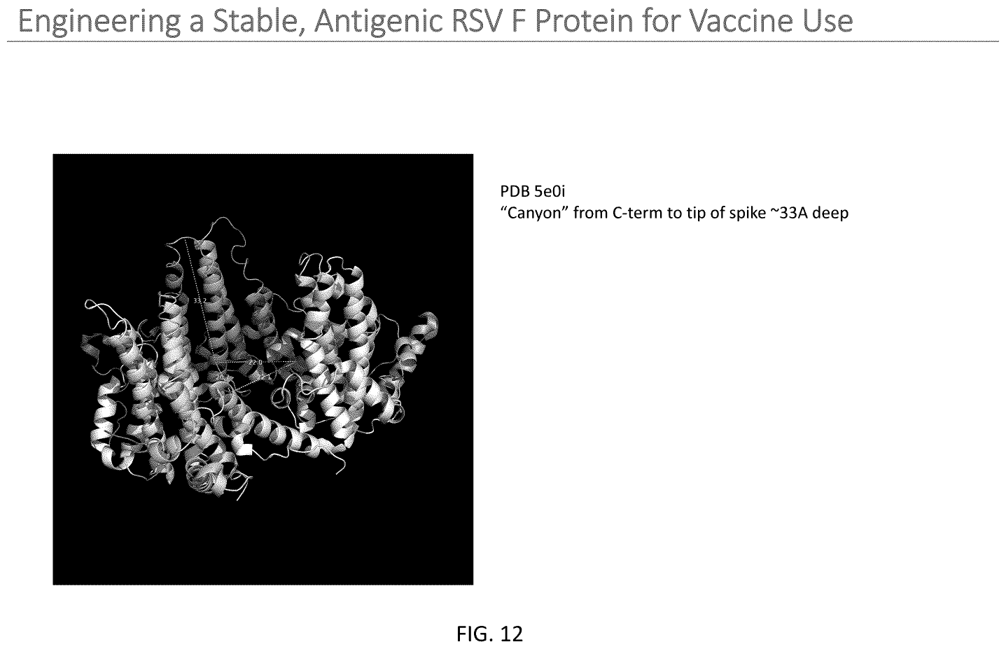

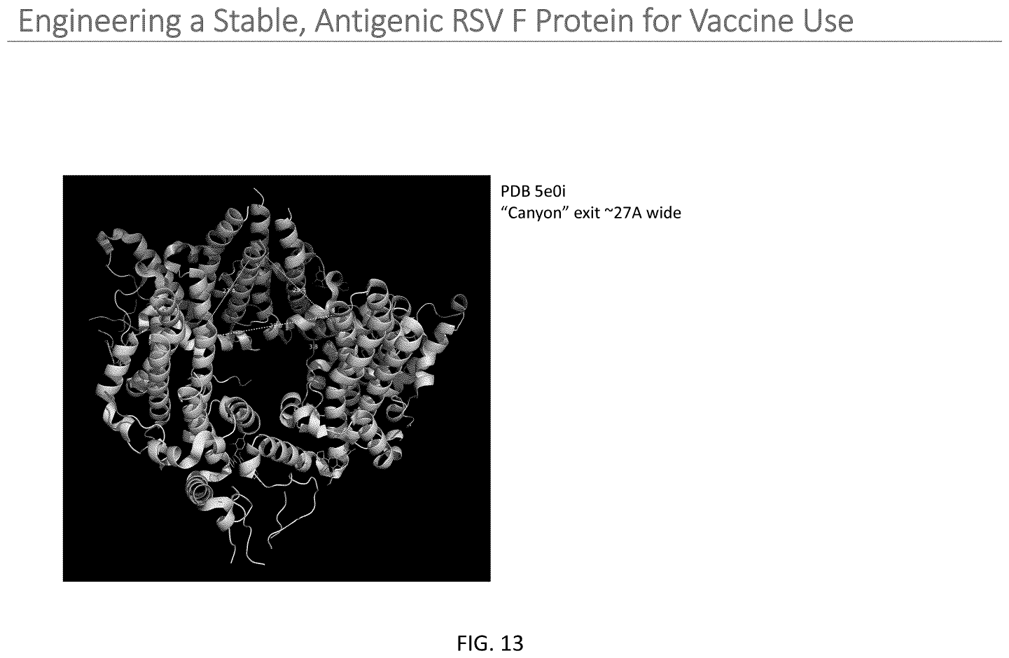

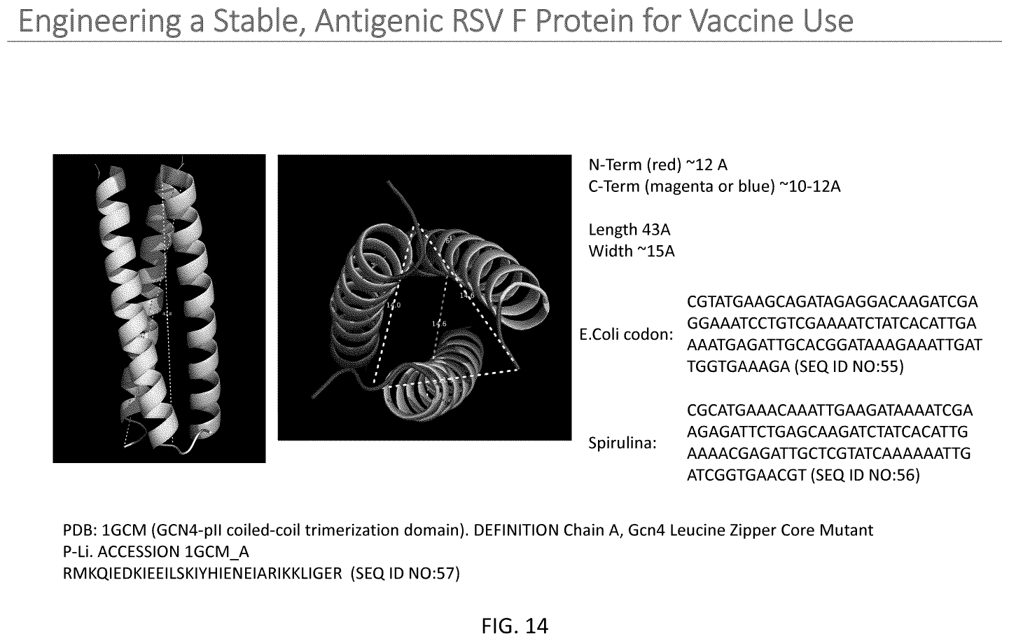

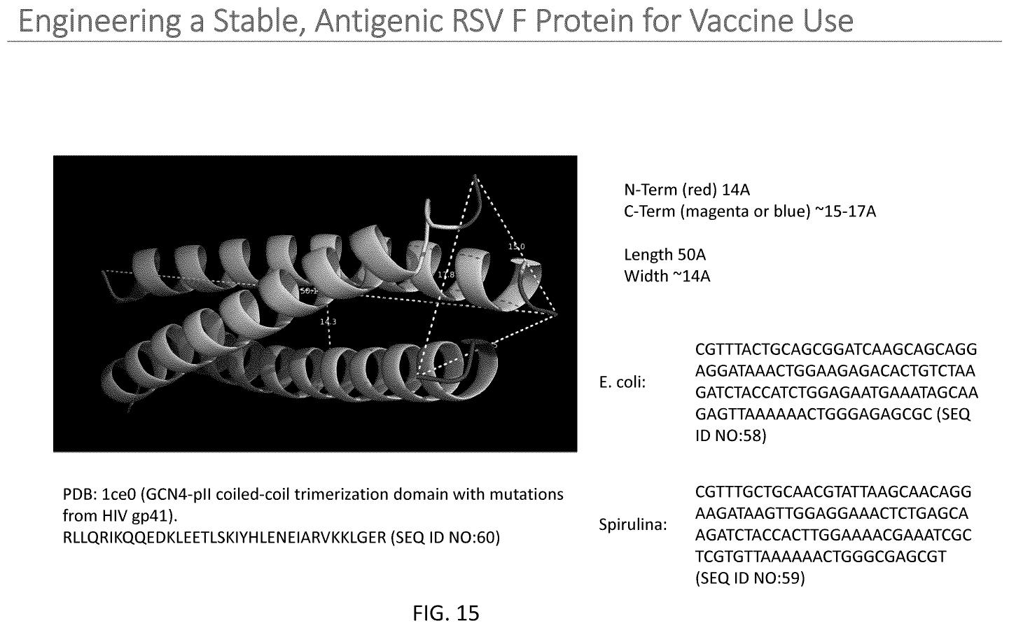

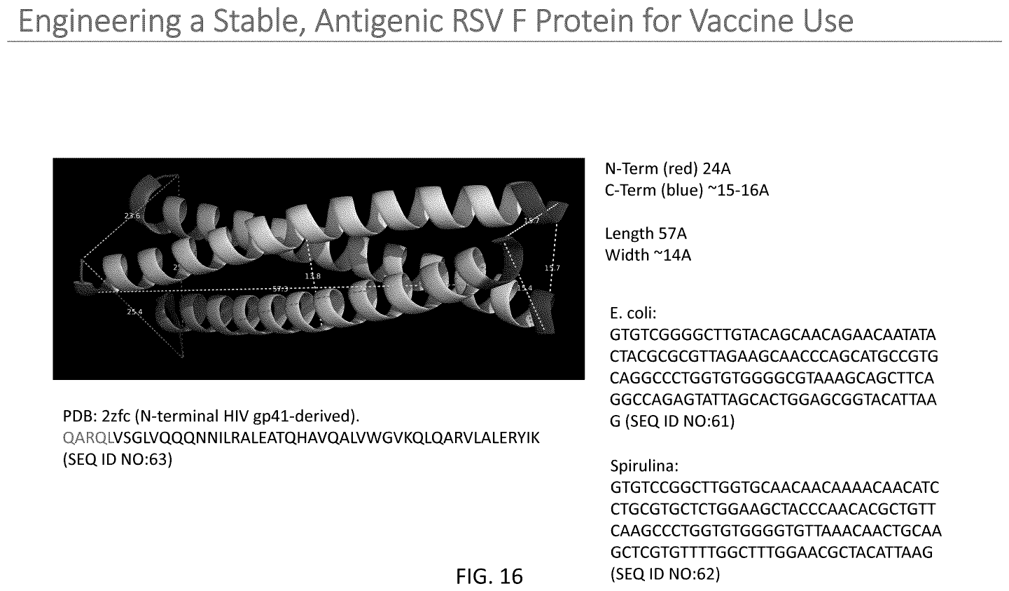

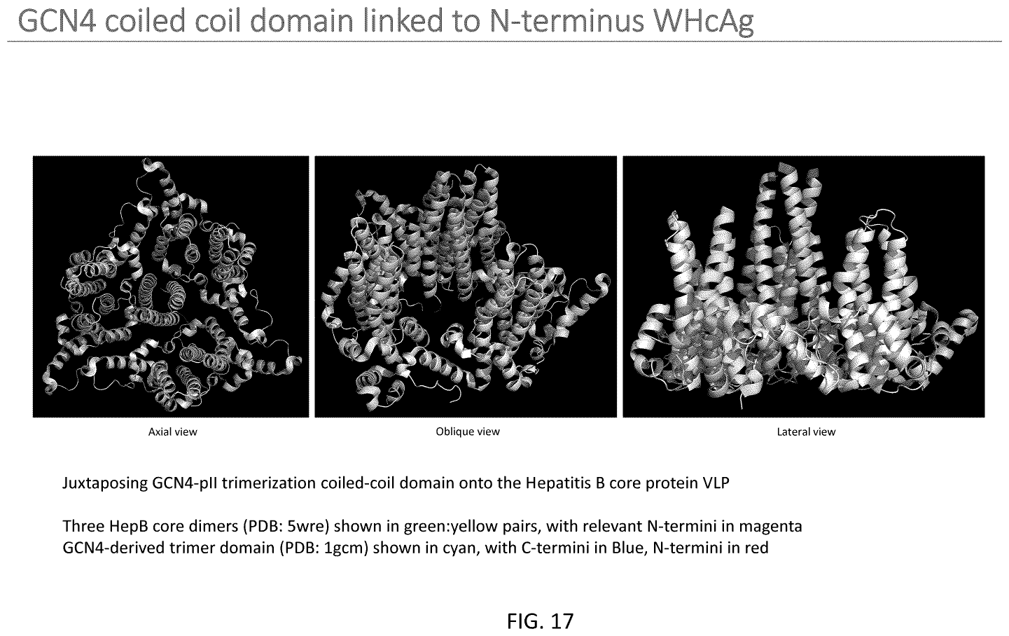

In an exemplary embodiment, as illustrated in FIGS. 11-17, the inventors have examined the geometry of the WHcAg VLP particle, and engineered the above-noted coiled coil structures to fit in the “canyon” between the spikes of the native WHcAg particle.

In some embodiments, the recombinant Spirulina comprises a fusion protein comprising at least one exogenous antigenic epitope and a viral protein capable of forming a virus-like particle (VLP). In these embodiments, the exogenous antigenic epitope is expressed in Spirulina as a protein macromolecular particle, such as virus-like particles (VLPs). VLPs mimic the overall structure of a virus particle by retaining the three-dimensional structure of a virus without containing infectious material. VLPs have the ability to stimulate B-cell and T-cell mediated responses. When viral proteins are expressed in a heterologous system, such as Spirulina, they can spontaneously form VLPs. Accordingly, in some embodiments, the at least one exogenous antigenic epitope is fused to a VLP-forming viral protein. When this fusion protein is expressed in Spirulina, it forms a VLP.

In some embodiments, tethering the exogenous antigenic epitope to a VLP-forming viral protein (or other protein that forms tertiary structures) allows the expression of hundreds of monomer proteins per VLP (e.g. 180-240 monomer proteins per VLP when using the hepatitis VLP). This allows the expression of thousands of millions of VLPs per cell. In some embodiments, the exogenous antigenic epitope is tethered to a VLP-forming viral protein. In some embodiments, the exogenous antigenic epitope is tethered to a VLP-forming viral protein at the C-terminus or the N-terminus of the viral protein. That is, the amino acid sequence for the antigen or the antigenic epitope is preceded by (attachment of the viral protein at the N-terminus of the antigen or the epitope), or followed by (attachment of the viral protein at the N-terminus of the antigen or the epitope), the amino acid sequence of the viral protein. In some other embodiments, the exogenous antigenic epitope is inserted into a VLP-forming viral protein. For example, the at least one exogenous antigenic epitope can be inserted between two adjacent amino acid residues of the viral protein. Alternatively, a region of the viral protein that is not required for the formation of a VLP can be replaced by inserting the at least one exogenous antigenic epitope in that region. Throughout this disclosure, when it is said that the at least one exogenous antigenic epitope is comprised in a VLP or is present in a VLP, it refers to the fusion protein comprising at least one exogenous antigenic epitope and a VLP-forming viral protein described herein.

Viral proteins that can be used to form antigenic epitope-containing VLPs of the present disclosure include capsid proteins of various viruses. Exemplary capsid proteins that can be used in the VLPs of the present disclosure include capsid proteins of viruses from the Hepadnaviridae family, papillomaviruses, picornaviruses, caliciviruses, rotaviruses, and reoviruses. In some embodiments, viral proteins that can be used to form antigen-or antigenic epitope-expressing VLPs of the present disclosure include the Hepadnaviridae core antigen (HBcAg). An exemplary HBcAg that can be used in the present disclosure is Woodchuck Hepadnaviral core antigen (WHcAg) from the Woodchuck Hepadnavirus (also referred to herein as Woodchuck Hepatitis Virus).

In some embodiments, the recombinant Spirulina comprises a fusion protein comprising at least one exogenous antigenic epitope and a protein that forms a trimer. In some embodiments, the trimer-forming protein is from an RNA bacteriophage or Helicobacter pylori. In some embodiments, the trimer-forming protein is the Helicobacter pylori ferritin protein. The at least one exogenous antigenic epitope can be attached at the C-terminus or the N-terminus, or within the body of the protein that forms a trimer. In some embodiments, these proteins that form a trimer include but are not limited to, GCN4 polypeptides from S. cerevisiae and/or HIV or fragments, mutants or variants thereof.

In some embodiments, the recombinant Spirulina comprises a fusion protein comprising at least one exogenous antigenic epitope and a scaffold protein. The term “scaffold protein” as used herein refers to a protein that acts as a docking protein and facilitates the interaction between two or more proteins. For example, a fusion protein comprising at least one exogenous antigenic epitope and a scaffold protein can facilitate the binding of the exogenous antigenic epitope with a receptor on an immune cell. In some embodiments, the exogenous antigenic epitope is tethered to a scaffold protein at the C-terminus or the N-terminus of the scaffold protein. In some other embodiments, the exogenous antigenic epitope is inserted into a scaffold protein (e.g. in the body of the scaffold protein). For example, the at least one exogenous antigenic epitope can be inserted between two adjacent amino acid residues of the scaffold protein. Alternatively, a region of the scaffold protein that is not required for the scaffolding function can be replaced by inserting the at least one antigenic epitope in that region. For example, in a recombinant Spirulina comprising multiple copies of the exogenous antigenic epitope and a scaffold protein, the exogenous antigenic epitope and the scaffold protein can be arranged in any one of the following patterns: (E)n-(SP), (SP)-(E)n, (SP)-(E)n-(SP), (E)n₁-(SP)-(E)n₂, (SP)-(E)n₁-(SP)-(E)n₂, and (SP)-(E)n₁-(SP)-(E)n₂-(SP), wherein E is the exogenous antigenic epitope. SP is the scaffold protein, and n, n₁, and n₂ represent the number of copies of the exogenous antigenic epitope. It is understood that the recombinant Spirulina may comprise more than one exogenous antigenic epitope and one or more scaffold proteins, where the multiple exogenous antigenic epitopes and the scaffold proteins can be arranged in various patterns as described above.

In some embodiments, recombinant Spirulina may comprise a fusion protein comprising at least one exogenous antigenic epitope, a scaffold protein, a VLP-forming viral protein, and/or a trimer-forming protein. In these embodiments, the at least one exogenous antigenic epitope can be tethered to or inserted into one or more scaffold proteins as described above and the fusion protein comprising the scaffold proteins and the at least one exogenous antigenic epitopes is tethered to or inserted into a VLP-forming viral protein and/or the trimer-forming protein.

Exemplary scaffold proteins include the oligomerization domain of C4b-binding protein (C4BP), a cholera toxin b subunit, or oligomerization domains of extracellular matrix proteins. In some embodiments, a scaffold protein used in the oral antigenic compositions of the present disclosure comprises a sequence from the oligomerization domain of C4BP selected from the group consisting of:

(SEQ ID NO: 1)
SAGAHAGWETPEGCEQVLTGKRLMQCLPNPEDVKMALEVYKLSLEIEQL
ELQRDSARQSTLDKEL,
(SEQ ID NO: 2)
WVIPEGCGHVLAGRKVMQCLPNPEDVKMALEVYKLSLEIELLEIQRDKA
RDPAMD,
(SEQ ID NO: 3)
WEYAEGCEQVVKGKKLMQCLPTPEEVRLALEVYKLYLEIQKLELQKDEA
KQA,
and
(SEQ ID NO: 4)
WVVPAGCEQVIAGRELTQCLPSVEDVKMALELYKLSLEIELLELQKDKA
KKSTLESPL.

The recombinant Spirulina present in the oral antigenic compositions of the present disclosure can comprise multiple copies of the at least one exogenous antigenic epitope. In some embodiments, the recombinant Spirulina expresses an exogenous antigen (natural antigen or a fusion protein as described above), wherein the exogenous antigen comprises at least 1, 2, 3, 4, 5, 6, 7, 8, 9, or 10 copies of the at least one exogenous antigenic epitope per single molecule of the exogenous antigen. In some embodiments, the recombinant Spirulina expresses an exogenous antigen, wherein the exogenous antigen comprises 1-5, 2-5, 2-4, 3-6, 3-8, or 4-5 copies of the at least one exogenous antigenic epitope per single molecule of the exogenous antigen. In some embodiments, the recombinant Spirulina comprises at least 1, 2, 3, 4, 5, 6, 7, 8, 9, 10, 15, 20, 25, 30, 35, 40, 45, 50, 55, or 60 copies of the at least one exogenous antigenic epitope per single molecule of the exogenous antigen. In some embodiments, the recombinant Spirulina expresses an exogenous antigen, wherein the exogenous antigen comprises 1-10, 1-15, 1-20, 1-25, 1-30, 1-40, 1-50, 5-10, 5-15, 5-20, 5-25, 5-30, 5-40, 5-50, 10-25, 10-50, 10-60, 15-30, 15-45, 15-60, 20-50, 20-60, 20-70, 25-50, 25-60, 30-60, or 2-100 copies of the at least one exogenous antigenic epitope per single molecule of the exogenous antigen. In some embodiments, the recombinant Spirulina cell can comprise thousands of copies of the at least one exogenous antigenic epitope (e.g. by expressing the corresponding nucleic acid sequences via one or more vectors in the cell or via integration into the Spirulina genome).

The recombinant Spirulina present in the oral antigenic compositions of the present disclosure can comprise multiple copies of a nucleic acid sequence encoding the at least one exogenous antigenic epitope. The multiple copies of the nucleic acid sequence encoding the at least one exogenous antigenic epitope can be integrated into the genome of the Spirulina or can be present on one or more vectors introduced into the Spirulina. In some embodiments, the recombinant Spirulina comprises between 2 and 100 copies of the nucleic acid sequence encoding the at least one exogenous antigenic epitope. In some embodiments, the recombinant Spirulina comprises at least 1, 2, 3, 4, 5, 6, 7, 8, 9, or 10 copies of a nucleic acid sequence encoding the at least one exogenous antigenic epitope integrated into its genome or present on one or more vectors. In some embodiments, the recombinant Spirulina comprises 1-5, 2-5, 2-4, 3-6, 3-8, or 4-5 copies of a nucleic acid sequence encoding the at least one exogenous antigenic epitope integrated into its genome or present on one or more vectors. In some embodiments, the recombinant Spirulina comprises at least 1, 2, 3, 4, 5, 6, 7, 8, 9, 10, 15, 20, 25, 30, 35, 40, 45, 50, 55, or 60 copies of a nucleic acid sequence encoding the at least one exogenous antigenic epitope integrated into its genome or present on one or more vectors. In some embodiments, the recombinant Spirulina comprises 1-10, 1-15, 1-20, 1-25, 1-30, 1-40, 1-50, 5-10, 5-15, 5-20, 5-25, 5-30, 5-40, 5-50, 10-25, 10-50, 10-60, 15-30, 15-45, 15-60, 20-50, 20-60, 20-70, 25-50, 25-60, or 30-60 copies of a nucleic acid sequence encoding the at least one exogenous antigenic epitope integrated into its genome or present on one or more vectors.

In some embodiments, multiple copies of the at least one exogenous antigenic epitope present, for example, in the exogenous antigen, are linked in tandem, i.e., the first copy is immediately followed by the second copy without being separated by any amino acids, the second copy is immediately followed by the third copy, and so on. For example, if NANP (SEQ ID NO: 6) represents an antigenic epitope and at least 4 copies of this epitope linked in tandem are present in an exogenous antigen expressed in the recombinant Spirulina, then the recombinant Spirulina comprises a protein or a peptide comprising a sequence of—NANPNANPNANPNANP-(SEQ ID NO: 5). In some embodiments, the repeating antigenic epitope can be comprised in an exogenous antigen expressed by recombinant Spirulina, where the exogenous antigen can be a natural antigen (e.g., CSP protein from Plasmodium), a fusion protein, or natural or fusion peptides. In some embodiments, where the recombinant Spirulina comprises more than one exogenous antigenic epitope, the individual antigenic epitope can be similarly linked in tandem to the other antigenic epitope. For example, in a recombinant Spirulina comprising E1 and E2 as exogenous antigenic epitopes, these two epitopes can be linked in tandem in the following ways: (E1E2)x, (E2E1)x, (E1)x(E2)y, (E1)x(E2)y(E1)z, (E2)x(E1)y(E2)z, where x, y, and z represent the number of copies of the epitopes. Similar arrangement patterns for more than two exogenous antigenic epitopes are contemplated.

In some embodiments, multiple copies of the at least one exogenous antigenic epitope present in a protein can be separated by spacer sequences. In some embodiments, multiple copies of the exogenous antigenic epitope can be separated by about 1 to about 50 amino acid space sequences. For example, in some embodiments, multiple copies of the exogenous antigenic epitope can be separated by about 1, 2, 3, 4, 5, 6, 7, 8, 9, 10, 15, 20, 30, 40, or 50 amino acid spacer sequences. It is understood that in these embodiments, when more than 2 copies of the exogenous antigenic epitope are present, some copies can be linked in tandem and some copies can be separated by spacer sequences. For example, in a recombinant Spirulina comprising multiple copies of E1 as the at least one exogenous antigenic epitope, the multiple copies of this epitope can be separated in the following ways: (E1)x-S-(E1)y, (E1)(E1)x-S-(E1)y, (E1)x-S-(E1)y-S-(E1)z, where S represents the spacer sequence and x, y, and z represent the number of copies of the exogenous epitope. When multiple spacer sequences are present, these sequences can be identical or different in length and/or the amino acid sequence.

In embodiments, where the recombinant Spirulina comprises a protein comprising more than one exogenous antigenic epitope, the first exogenous antigenic epitope can be separated from the other exogenous antigenic epitope by spacer sequences of about 1, 2, 3, 4, 5, 6, 7, 8, 9, 10, 15, 20, 30, 40, or 50 amino acids. If multiple copies of each of the exogenous antigenic epitopes are present, some of the copies can be linked in tandem with the other epitope while some copies can be separated by spacer sequences: alternatively, all copies of one epitope can be linked in tandem followed by a spacer sequence followed by all copies of the second epitope, and the like. For example, in a recombinant Spirulina comprising E1 and E2 as exogenous antigenic epitopes, the two epitopes can be arranged in the following ways: (E1)x-S-(E2)y, (E2)x-S-(E1)y, (E1)x-S-(E2)y-S(E1)z-S-(E2)v, (E1)x-S-(E2)y(E1)z, (E1)x-S-(E2)y-S-(E1)z, (E2)x-S-(E1)y(E2)z, and the like, where v, x, y, and z represent the number of copies of the epitopes.

In some embodiments, a recombinant Spirulina may comprise one or more exogenous antigenic epitopes and multiple copies thereof in the arrangement patterns described above directly, i.e., without being part of or fused to another protein. In some embodiments, the one or more exogenous antigenic epitopes can be from the same antigen. In some embodiments, the one or more exogenous antigenic epitopes can be from different antigens. In some other embodiments, one or more exogenous antigenic epitopes and multiple copies thereof can be comprised in an exogenous antigen in the arrangement patterns described above. The exogenous antigen can be a natural antigen or a fusion protein as discussed above.

In some embodiments, the exogenous antigenic epitopes can be from different antigens that activate different types of immunity (e.g. innate, cellular, or humoral). In some embodiments, the one or more exogenous antigenic epitopes from different antigens are from at least one B-cell antigen and at least one T-cell antigen. In some embodiments, the one or more exogenous antigenic epitopes are in a fusion protein with a viral protein (e.g. a coronavirus spike protein). In some embodiments, the one or more exogenous antigenic epitopes are in a fusion protein with a viral protein (e.g. a coronavirus spike protein) with one epitope at either terminus. In some embodiments, the one or more exogenous antigenic epitopes are a B-cell epitope fused to one terminus of a virus protein and a T-cell epitope fused to the other terminus of the virus protein.

In some embodiments, recombinant Spirulina comprises a fusion protein comprising a VLP-forming viral protein or a trimer-forming protein and one or more exogenous antigenic epitopes, where the exogenous antigenic epitopes and multiple copies thereof, if present, can be arranged within the fusion protein in various patterns as described above. In some other embodiments, recombinant Spirulina may comprise a fusion protein comprising a scaffold protein and one or more exogenous antigenic epitopes, where the exogenous antigenic epitopes and multiple copies thereof, if present, can be arranged within the fusion protein in various patterns as described above. In some other embodiments, recombinant Spirulina may comprise a fusion protein comprising a VLP-forming viral protein, a trimer-forming protein, and/or a scaffold protein, and one or more exogenous antigenic epitopes, where the exogenous antigenic epitopes and multiple copies thereof, if present, can be arranged within the fusion protein in various patterns as described above.

The oral antigenic compositions provided by the present disclosure comprise a recombinant Spirulina, wherein the recombinant Spirulina comprises at least one exogenous antigenic epitope in any of the ways described above.

In various embodiments, oral antigenic compositions of the present disclosure comprise a recombinant Spirulina comprising at least one exogenous antigenic epitope derived from (e.g. a portion or fragment thereof, or antigenic variant thereof) an infectious microorganism, a tumor antigen or a self-antigen associated with an autoimmune disease.

In some embodiments, oral antigenic compositions comprise a recombinant Spirulina comprising at least one exogenous antigenic epitope derived from an infectious microorganism such as a virus, bacterium, parasite, or fungus. The infectious microorganism can be a microorganism that causes infections in a human or an animal such as a species of livestock, poultry, and fish.

In some embodiments, oral antigenic compositions of the present disclosure comprise a recombinant Spirulina comprising at least one antigenic epitope from a virus including but not limited to, bacteriophage, RNA bacteriophage (e.g. MS2, AP205, PP7 and Qβ), Helicobacter pylori, infectious haematopoietic necrosis virus (IHNV), parvovirus, Herpes Simplex Virus, Hepatitis A virus, Hepatitis B virus, Hepatitis C virus, Measles virus, Mumps virus, Rubella virus, Human Immunodeficiency Virus (HIV), Influenza virus, Rhinovirus, Rotavirus A, Rotavirus B, Rotavirus C, Respiratory Syncytial Virus (RSV), Varicella zoster, Poliovirus, Norovirus, Zika Virus, Denge Virus, Rabies Virus, Newcastle Disease Virus, and White Spot Syndrome Virus. In some embodiments, oral antigenic compositions of the present disclosure comprise a recombinant Spirulina comprising at least one antigenic epitope from IHNV. In some embodiments, oral antigenic compositions of the present disclosure comprise a recombinant Spirulina comprising at least one antigenic epitope from a parvovirus, e.g., canine parvovirus.

In some embodiments, oral antigenic compositions comprise a recombinant Spirulina comprising at least one antigenic epitope from a bacterium including but not limited to, Mycobacterium, Streptococcus, Staphylococcus, Shigella, Campylobacter, Salmonella, Clostridium, Corynebacterium, Pseudomonas, Neisseria, Listeria, Vibrio, Bordetella, E. coli (including pathogenic E. coli), and Legionella.

In some embodiments, oral antigenic compositions comprise a recombinant Spirulina comprising at least one antigenic epitops from a parasite including but not limited to, Plasmodium, Trypanosoma, Toxoplasma, Giardia, and Leishmania, Cryptosporidium, helminthic parasites: Trichuris spp. (whipworms), Enterobius spp. (pinworms), Ascaris spp. (roundworms), Ancylostoma spp. and Necator spp. (hookworms), Strongyloides spp. (threadworms), Dracunculus spp. (Guinea worms), Onchocerca spp. and Wuchereria spp. (filarial worms), Taenia spp., Echinococcus spp., and Diphyllobothrium spp. (human and animal cestodes), Fasciola spp. (liver flukes) and Schistosoma spp. (blood flukes).

In some embodiments, oral antigenic compositions comprise a recombinant Spirulina comprising at least one antigenic epitope from a Plasmodium selected from the group consisting of: P. falciparum, P. malariae, P. ovale and P. vivax.

In some embodiments, oral antigenic compositions comprise a recombinant Spirulina comprising at least one antigenic epitope from a fungus including but not limited to, Aspergillus, Candida, Blastomyces, Coccidioides, Cryptococcus, and Histoplasma. In some embodiments, oral antigenic compositions comprise a recombinant Spirulina comprising at least one antigenic epitope from Candida albicans or Candida auris.

In some embodiments, provided herein are oral antigenic compositions comprising a recombinant Spirulina, wherein the recombinant Spirulina comprises at least one antigenic epitope from a Plasmodium. In some embodiments, provided herein are oral antigenic composition comprising a recombinant Spirulina, wherein the recombinant Spirulina comprises at least one antigenic epitope derived from a Plasmodium antigen selected from the group consisting of: circumsporozoite protein (CSP or CS), thrombospondin-related anonymous protein (TRAP), Apical Membrane Antigen 1 (AMA1), the major merozoite surface proteins 1-3 (MSP1-3), sexual stage antigen 25 (s25), and sexual stage antigen s230. In some embodiments, the at least one Plasmodium antigenic epitope is comprised in a VLP. In some embodiments, the VLP comprises the sequence of a capsid protein of a virus. In an exemplary embodiment, the capsid protein is woodchuck hepadnaviral core antigen (WHcAg).

In some embodiments, provided herein are oral antigenic compositions comprising a recombinant Spirulina, wherein the recombinant Spirulina comprises at least one antigenic epitope derived from a circumsporozoite protein of Plasmodium. In some embodiments, provided herein are oral antigenic compositions comprising a recombinant Spirulina, wherein the recombinant Spirulina comprises a VLP containing at least one antigenic epitope derived from a circumsporozoite protein of Plasmodium. In some embodiments, the VLP comprises the sequence of a capsid protein of a virus. In an exemplary embodiment, the capsid protein is woodchuck hepadnaviral core antigen (WHcAg).

In some embodiments, oral antigenic compositions of the present disclosure comprise a recombinant Spirulina, wherein the recombinant Spirulina comprises one or more antigenic epitopes from Table 1 or a fusion protein comprising an epitope-containing sequence selected from Table 1.

TABLE 1

| | | SEQ |
|---|---|---|
| Epitope | Epitope containing sequence | ID NO |
| P. falciparum CSP | NANP | 6 |
| P. falciparum CSP | NVDP | 7 |
| P. falciparum CSP | NPDP | 8 |
| P. falciparum CSP | KLKQPGDGNPDPNANPNVDPNANPNVDPNA | 9 |
| | NPNVDPNANP | |
| P. falciparum CSP | EYLKKIQNSLSTEWSPCSVT | 10 |
| P. falciparum CSP | NANPNANPNANPNANPNANPNANPNANPNA | 11 |
| | NPNANPNANPNANPNANPNANPNANPNANP | |
| P. falciparum CSP | GDGNPDPNANPNVDPNANPNVDPNANPNVD | 12 |
| | PNANP | |
| P. falciparum CSP | GDGNPDPNANPNVDPNANPNVDPNANPNVDP | 13 |
| | NANPNANPNANPNANPNANPNANPNANPNA | |
| | NPNANPNANPNANPNANPNANPNANPNANP | |
| P. falciparum CSP | DGNNEDNEKLRKPKHKKLKQPGDGNPDPNA | 14 |
| | NPNVDPNANPNVDPNANPNVDPNANPNANPN | |
| | ANPNANPNANPNANPNANPNANPNANPNANP | |
| | NANPNANPNANPNANPNANP | |
| P. falciparum | HPSDGKC | 15 |
| TRAP | | |
| P. falciparum | DRYI | 16 |
| TRAP | | |
| P. falciparum | TRPHGR | 17 |
| TRAP | | |
| P. yoelii CSP | QGPGAPQGPGAPQGPGAPQGPGAPQGPGAPQ | 18 |
| | GPGAPQEPPQQPPQQPPQQPPQQPPQQ | |
| P. yoelii CSP | NEDSYVPSAEQILEF | 19 |
| P. falciparum CSP | EEGNANPNANPNANPNANPNANPNANPNANP | 20 |
| | NANPNANPNANPNANPNANPNANPNANPNA | |
| | NPGEE | |
| Canine parvovirus | SDGAVQPDGGQPAVRNERATG | 21 |
| 2L21 peptide | | |
| epitope | | |
| Canine parvovirus | DGAVQPDGGQPAVRNER | 22 |
| 3L17 peptide | | |
| epitope | | |
| IHNV E1 + E2 | DDENRGLIAYPTSIRSLSVGGSGGSDLISVVYN | 23 |
| epitopes | SGSEILSF | |
| IHNV DIII epitope | DDENRGLIAYPTSIRSLSVGNDGGSGGSSQEIK | 24 |
| | AHLFVDKISNRVVKATSYGHHPWGLHQACMI | |
| | EFCGQQWIRTDLGDLISVVYNSGSEILSFPKCE | |
| | DKTVGPAEGGG | |
| IHNV DIII epitope | DDENRGLIAYPTSIRSLSVGNDGGSGGSSQEIK | 64 |
| | GHLFVDKISNRVVKATSYGHHPWGLHQACMI | |
| | EFCGQQWIRTDLGDLISVVYNSGSEILSFPKCE | |
| | DKTVG | |
| HIV gp41 fusion | AVGIGAVF | 65 |
| peptide A epitope | | |
| HIV gp41 fusion | AVGLGAVF | 66 |
| peptide B epitope | | |
| HIV gp41 fusion | AVGIGAMI | 67 |
| peptide C epitope | | |

In some embodiments, oral antigenic compositions of the present disclosure comprise a recombinant Spirulina, wherein the recombinant Spirulina comprises a fusion protein comprising one or more antigenic epitopes, wherein the fusion protein comprises a sequence selected from Table 2.

TABLE 2

| Description of the | | |
|---|---|---|
| fusion protein | Sequence | SEQ ID NO |
| P. yoelii CSP B cell | MDIDPYKEFGSSYQLLNFLPLDFFPDLNALVD | 25 |
| epitope in monomer | TATALYEEELTGREHCSPHHTAIRQALVCWD | |
| WHcAg | ELTKLIAWMSSNITSQGPGAPQGPGAPQGPG | |
| | APQGPGAPQGPGAPQGPGAPQEPPQQPPQQP | |
| | PQQPPQQPPQQEQVRTIIVNHVNDTWGLKVR | |
| | QSLWFHLSCLTFGQHTVQEFLVSFGVWIRTP | |
| | APYRPPNAPILSTLPEHTVINEDSYVPSAEQIL | |
| | EFEQKLISEEDLEQKLISEEDL | |
| P. falciparum CSP | MDIDPYKEFGSSYQLLNFLPLDFFPDLNALVD | 26 |
| B cell epitope in | TATALYEEELTGREHCSPHHTAIRQALVCWD | |
| tandem WHcAg | ELTKLIAWMSSNITSEQVRTIIVNHVNDTWGL | |
| dimer | KVRQSLWFHLSCLTFGQHTVQEFLVSFGVWI | |
| | RTPAPYRPPNAPILSTLPEHTVIGGSGGSGGSG | |
| | GSGGSDIDPYKEFGSSYQLLNFLPLDFFPDLN | |
| | ALVDTATALYEEELTGREHCSPHHTAIRQAL | |
| | VCWDELTKLIAWMSSNITSEEGNANPNANPN | |
| | ANPNANPNANPNANPNANPNANPNANPNAN | |
| | PNANPNANPNANPNANPNANPGEEEQVRTII | |
| | VNHVNDTWGLKVRQSLWFHLSCLTFGQHTV | |
| | QEFLVSFGVWIRTPAPYRPPNAPILSTLPEHTV | |
| | IHHHHHH | |
| P. falciparum CSP | MDIDPYKEFGSSYQLLNFLPLDFFPDLNALVD | 27 |
| B cell epitope + | TATALYEEELTGREHCSPHHTAIRQALVCWD | |
| Salmonella fliC | ELTKLIAWMSSNITSEQVRTIIVNHVNDTWGL | |
| sequences in tandem | KVRQSLWFHLSCLTFGQHTVQEFLVSFGVWI | |
| WHcAg dimer | RTPAPYRPPNAPILSTLPEHTVIGGSGGSGGSG | |
| | GSGGSDIDPYKEFGSSYQLLNFLPLDFFPDLN | |
| | ALVDTATALYEEELTGREHCSPHHTAIRQAL | |
| | VCWDELTKLIAWMSSNITSEEGAQVINTNSLS | |
| | LLTQNNLNKSQSALGTAIERLSSGLRINSAKD | |
| | DAAGQAIANRFTANIKGLTQASRNANDGISIA | |
| | QTTEGALNEINNNLQRVRELAVQSANSTNSQ | |
| | SDLDSIQAEITQRLNEIDRVSGQTQFNGVKVL | |
| | AQDNTLTIQVGANDGETIDIDLKQINSQTLGL | |
| | DTLNVQGSNANPNANPNANPNANPNANPNA | |
| | NPNANPNANPNANPNANPNANPNANPNANP | |
| | NANPNANPGSTTTENPLQKIDAALAQVDTLR | |
| | SDLGAVQNRFNSAITNLGNTVNNLTSARSRIE | |
| | DSDYATEVSNMSRAQILQQAGTSVLAQANQ | |
| | VPQNVLSLLRGEEEQVRTIIVNHVNDTWGLK | |
| | VRQSLWFHLSCLTFGQHTVQEFLVSFGVWIR | |
| | TPAPYRPPNAPILSTLPEHTVIHHHHHH | |
| Canine parvovirus | MDIDPYKEFGSSYQLLNFLPLDFFPDLNALVD | 28 |
| 2L21 peptide | TATALYEEELTGREHCSPHHTAIRQALVCWD | |
| epitope in 2x | ELTKLIAWMSSNITSEEGSDGAVQPDGGQPA | |
| configuration; | VRNERATGGGSSDGAVQPDGGQPAVRNERA | |
| monomer WhcAg | TGGEEEQVRTIIVNHVNDTWGLKVRQSLWFH | |
| | LSCLTFGQHTVQEFLVSFGVWIRTPAPYRPPN | |
| | APILSTLPEHTVIHHHHHH | |
| Canine parvovirus | MDIDPYKEFGSSYQLLNFLPLDFFPDLNALVD | 29 |
| 3L17 peptide | TATALYEEELTGREHCSPHHTAIRQALVCWD | |
| epitope in 4x | ELTKLIAWMSSNITSEEGDGAVQPDGGQPAV | |
| configuration; | RNERGGSDGAVQPDGGQPAVRNERGGSDGA | |
| monomer WhcAg | VQPDGGQPAVRNERGGSDGAVQPDGGQPAV | |
| | RNERGEEEQVRTIIVNHVNDTWGLKVRQSL | |
| | WFHLSCLTFGQHTVQEFLVSFGVWIRTPAPY | |
| | RPPNAPILSTLPEHTVIHHHHHH | |
| IHNV E1 + E2 | MDIDPYKEFGSSYQLLNFLPLDFFPDLNALVD | 30 |
| epitopes in tandem | TATALYEEELTGREHCSPHHTAIRQALVCWD | |
| WHcAg dimer | ELTKLIAWMSSNITSEQVRTIIVNHVNDTWGL | |
| | KVRQSLWFHLSCLTFGQHTVQEFLVSFGVWI | |
| | RTPAPYRPPNAPILSTLPEHTVIGGSGGSGGSG | |
| | GSGGSDIDPYKEFGSSYQLLNFLPLDFFPDLN | |
| | ALVDTATALYEEELTGREHCSPHHTAIRQAL | |
| | VCWDELTKLIAWMSSNITSPGGSGDDENRGL | |
| | IAYPTSIRSLSVGGSGGSDLISVVYNSGSEILSF | |
| | PGGSGEQVRTIIVNHVNDTWGLKVRQSLWFH | |
| | LSCLTFGQHTVQEFLVSFGVWIRTPAPYRPPN | |
| | APILSTLPEHTVIEQKLISEEDLEQKLISEEDL | |
| IHNV DIII epitope | MDIDPYKEFGSSYQLLNFLPLDFFPDLNALVD | 31 |
| in tandem WHcAg | TATALYEEELTGREHCSPHHTAIRQALVCWD | |
| dimer | ELTKLIAWMSSNITSEQVRTIIVNHVNDTWGL | |
| | KVRQSLWFHLSCLTFGQHTVQEFLVSFGVWI | |
| | RTPAPYRPPNAPILSTLPEHTVIGGSGGSGGSG | |
| | GSGGSDIDPYKEFGSSYQLLNFLPLDFFPDLN | |
| | ALVDTATALYEEELTGREHCSPHHTAIRQAL | |
| | VCWDELTKLIAWMSSNITSPGGSGDDENRGL | |
| | IAYPTSIRSLSVGNDGGSGGSSQEIKAHLFVD | |
| | KISNRVVKATSYGHHPWGLHQACMIEFCGQ | |
| | QWIRTDLGDLISVVYNSGSEILSFPKCEDKTV | |
| | GPAEGGGPAGGSGEQVRTIIVNHVNDTWGLK | |
| | VRQSLWFHLSCLTFGQHTVQEFLVSFGVWIR | |
| | TPAPYRPPNAPILSTLPEHTVIEQKLISEEDLEQ | |
| | KLISEEDL | |

In some embodiments, oral antigenic compositions of the present disclosure comprise a recombinant Spirulina, wherein the recombinant Spirulina expresses a protein comprising a sequence selected from Table 3. In some embodiments, recombinant Spirulina expresses a fusion protein comprising one or more antigenic epitopes from the proteins listed in Table 3.

TABLE 3
P. falciparum CSP sequences

| CSP Accession | |
|---|---|
| Number | CSP sequence |
| FJ232362 | MRKLAILSVSSFLFVEALFQEYQCYGSSSNTRVLNELNYDNAGTNLY |
| | NELEMNYYGKQENWYSLKKNSRSLGENDDGNNNNGDNGREGKDE |
| | DKRDGNNEDNEKLRKPKHKKLKQPGDGNPDPNANPNVDPNANPNV |
| | DPNANPNVDPNANPNANPNANPNANPNANPNANPNANPNANPNANP |
| | NANPNANPNANPNANPNANPNANPNANPNANPNANPNANPNANPN |
| | ANPNANPNANPNANPNANPNANPNANPNANPNANPNANPNANPNA |
| | NPNANPNANPNANPNANPNANPNANPNANPNANPNKNNQGNGQGH |
| | NMPNDPNRNVDENANANNDVKNNNNEEPSDKHIEEYLKKIQNSLSTE |
| | WSPCSVTCGNGIQVRIKPGSANKPKDELNYENDIEKKICKMEKCSSVF |
| | NVV NSSIGLIMVLSFLFLN (SEQ ID NO: 32) |
| GQ890770 | MRKLAILSVSSFLFVEALFQEYQCYGSSSNTRVLNELNYDNAGTNLY |
| | NELEMNYYGKQENWYSLKKNSRSLGENDDGNNNNGDNGREGKDE |
| | DKRDGNNEDNEKLRKPKHKKLKQPGDGNPDPNANPNVDPNANPNV |
| | DPNANPNVDPNANPNANPNANPNANPNANPNANPNANPNANPNANP |
| | NANPNANPNANPNANPNANPNANPNVDPNANPNANPNANPNANPN |
| | ANPNANPNANPNANPNANPNANPNANPNANPNANPNANPNANPNA |
| | NPNANPNANPNANPNANPNANPNANPNANPNANPNANPNKNNQGN |
| | GQGHNMPNDPNRNVDENANANNDENNNNEEPSDKHIEQYLKKIQY |
| | SLSTEWSPCSVTCGNGIQVRIKPGSADKPKDQLDYENDIEKKICKMEK |
| | CSSVFNVV NSSIGLIMVLSFLFLN (SEQ ID NO: 33) |
| FJ232364 | MRKLAILSVSSFLFVEALFQEYQCYGSSSNTRVLNELNYDNAGTNLY |
| | NELEMNYYGKQENWYSLKKNSRSLGENDDGNNNNGDNGREGKDE |
| | DKRDGNNEDNEKLRKPKHKKLKQPGDGNPDPNANPNVDPNANPNV |
| | DPNANPNVDPNANPNANPNANPNANPNANPNANPNANPNANPNANP |
| | NANPNANPNANPNANPNANPNANPNANPNANPNANPNANPNANPN |
| | ANPNANPNANPNANPNANPNANPNANPNANPNANPNANPNANPNA |
| | NPNANPNANPNANPNANPNANPNANPNKNNQGNGQGHNMPNDPNR |
| | NVDENANANNAVKNNNNEEPSDKHIEQYLKKIQNSLSTEWSPCSVTC |
| | GNGIQVRIKPGSANKPKDELDYENDIEKKICKMEKCSSVFNVVNSSIG |
| | LIMVLSFLFLN (SEQ ID NO: 34) |
| M22982.1 | MMRKLAILSVSSFLFVEALFQEYQCYGSSSNTRVLNELNYDNAGTNL |
| | YNELEMNYYGKQENWYSLKKNSRSLGENDDGNNEDNEKLRKPKH |
| | KKLKQPADGNPDPNANPNVDPNANPNVDPNANPNVDPNANPNANP |
| | NANPNANPNANPNANPNANPNANPNANPNANPNANPNANPNANPN |
| | ANPNANPNANPNANPNANPNANPNVDPNANPNANPNANPNANPNA |
| | NPNANPNANPNANPNANPNANPNANPNANPNANPNANPNANPNAN |
| | PNANPNANPNKNNQGNGQGHNMPNDPNRNVDENANANSAVKNNN |
| | NEEPSDKHIKEYLNKIQNSLSTEWSPCSVTCGNGIQVRIKPGSANKPK |
| | DELDYANDIEKKICKMEKCSSVFNVVNSSIGLIMVLSFLFLN (SEQ ID |
| | NO: 35) |
| FJ232355 | MRKLAILSVSSFLFVEALFQEYQCYGSSSNTRVLNELNYDNAGTNLY |
| | NELEMNYYGKQENWYSLKKNSRSLGENDDGNNNNGDNGREGKDED |
| | KRDGNNEDNEKLRKPKHKKLKQPGDGNPDPNANPNVDPNANPNVDP |
| | NANPNVDPNANPNANPNANPNANPNANPNANPNANPNANPNANPN |
| | ANPNANPNANPNANPNANPNANPNANPNANPNANPNANPNANPNA |
| | NPNANPNANPNANPNANPNANPNANPNANPNANPNANPNANPNAN |
| | PNANPNANPNANPNANPNANPNANPNANPNKNNQGNGQGHNMPND |
| | PNRNVDENANANNAVKNNNNEEPSDKHIEQYLKKIQNSLSTEWSPCS |
| | VTCGNGIQVRIKPGSANKPKDELDYENDIEKKICKMEKCSSVFNVV |
| | NSSIGLIMVLSFLFLN (SEQ ID NO: 36) |
| AB502949 | MMRKLAILSVSSFLFVEALFQEYQCYGSSSNTRVLNELNYDNAGTNL |
| | YNELEMNYYGKQENWYSLKKNSRSLGENDDGNNNNGDNGREGKDE |
| | DKRDGNNEDNEKLRKPKHKKLKQPGDGNPDPNANPNVDPNANPNV |
| | DPNANPNVDPNANPNVDPNANPNANPNANPNANPNANPNANPNANP |
| | NANPNANPNANPNANPNANPNANPNANPNANPNANPNANPNANPN |
| | ANPNANPNANPNANPNANPNANPNANPNANPNANPNANPNANPNA |
| | NPNANPNANPNANPNANPNANPNANPNANPNKNNQGNGQGHNMPN |
| | DPNRNVDENANANNAVKNNNNEEPSDKHIEQYLKKIQNSLSTEWSPC |
| | SVTCGNGIQVRIKPGSANKPKDELDYENDIEKKICKMEKCSSVFNVV |
| | NSSIGLIMVLSFLFLN (SEQ ID NO: 37) |
| AB715565 | MMRKLAILSVSSFLFVEALFQEYQCYGSSSNTRVLNELNYDNAGTNL |
| | YNELEMNYYGKQENWYSLKKNSRSLGENDDGNNNNGDNGREGKD |
| | EDKRDGNNEDNEKLRKPKHKKLKQPGDGNPDPNANPNVDPNANPN |
| | VDPNANPNVDPNANPNANPNANPNANPNANPNANPNANPNANPNA |
| | NPNANPNANPNANPNANPNANPNANPNANPNANPNANPNANPNAN |
| | PNANPNANPNANPNANPNANPNANPNANPNANPNANPNANPNANPN |
| | ANPNANPNANPNANPNANPNKNNQGNGQGHNMPNDPNRNVDENAN |
| | ANNAVKNNNNEEPSDKHIEQYLKKIQNSLSTEWSPCSVTCGNGIQVRI |
| | KPGSANKPKDELDYENDIEKKICKMEKCSSVFNVV NSSIGLIMVLSFL |
| | FLN (SEQ ID NO: 38) |
| AB116603 | MMRKLAILSVSSFLFVEALFQEYQCYGSSSNTRVLNELNYDNAGTNL |
| | YNELEMNYYGKQENWYSLKKNSRSLGENDDGNNNNGDNGREGKDE |
| | DKRDGNNEDNEKLRKPKHKKLKQPGDGNPDPNANPNVDPNANPNV |
| | DPNANPNVDPNANPNANPNANPNANPNANPNANPNANPNANPNANP |
| | NANPNANPNANPNANPNANPNANPNANPNANPNANPNANPNANPN |
| | ANPNANPNANPNANPNANPNANPNANPNANPNANPNANPNANPNA |
| | NPNANPNANPNANPNANPNKNNQGNGQGHNMPNDPNRNVDENAN |
| | ANNAVKNNNNEEPSDKHIEQYLKKIQNSLSTEWSPCSVTCGNGIQV |
| | RIKPGSANKPKDELDYENDIEKKICKMEKCSSVFNVV NSSIGLIMVL |
| | SFLFLN (SEQ ID NO: 39) |
| AB503046 | MMRKLAILSVSSFLFVEALFQEYQCYGSSSNTRVLNELNYDNAGTNL |
| | YNELEMNYYGKQENWYSLKKNSRSLGENDDGNNNNGDNGREGKD |
| | EDKRDGNNEDNEKLRKPKHKKLKQPGDGNPDPNANPNVDPNANP |
| | NVDPNANPNVDPNANPNANPNANPNANPNANPNANPNANPNANPN |
| | ANPNANPNANPNANPNANPNANPNANPNVDPNANPNANPNANPN |
| | ANPNANPNANPNANPNANPNANPNANPNANPNANPNANPNANPNA |
| | NPNANPNANPNANPNANPNKNNQGNGQGHNMPNDPNRNVDENANA |
| | NNAVKNNNNEEPSDKHIEQYLKKIQNSLSTEWSPCSVTCGNGIQVRIK |
| | PGSADKPKDQLDYENDIEKKICKMEKCSSVFNVVNSSIGLIMVLSFLF |
| | LN (SEQ ID NO: 40) |

P. falciparum TRAP sequences

| TRAP | |
|---|---|
| accession | |
| number | TRAP Sequence |
| AB807859 | MNHLGNVKYLVIVFLIFFDLFLVNGRDVQNNIVDEIKYREEVCNDEV |
| | DLYLLMDCSGSIRRHNWVKHAVPLAMKLIQQLNLNENAIHLYLNIFS |
| | NAREIIRLHSDASKNKEKALIIIKSLLNTNLPYGRTNLTDALLQVRKHL |
| | NDRINRENANQLVVILTDGIPDSIQDSLKESRKLNDRGVKIAVFGIGQG |
| | INVAFNRFLVGCHPSDGKCNLYADSAWENVKNVIGPFMKAVCVEVE |
| | KTASCGVWDEWSPCSVTCGKGTRSRKREILHEGCTSELQEQCEEERC |
| | PPKREPLDVPDEPEDDQPRPRGDNFAVEKPEENIIDNNPQEPSPNPEEG |
| | KGENPNGFDLDENPENPPNPPNPPNPDIPEQEPNIPEDSEKEVPSDVPK |
| | NPEDDREENFDIPKKPENKHDNQNNLPNDKSDRYIPYSPLPPKVLDNE |
| | RKQSDPQSQDNNGNRHVPNSEDRETRPHGRNNENRSYNRKHNNTPK |
| | HPEREEHEKPDNNKKKGGSDNKYKIAGGIAGGLALLACAGLAYKFV |
| | VPGAATPYAGEPAPFDETLGEEDKDLDEPEQFRLPEENEWN (SEQ ID |
| | NO: 41) |
| AB807845 | MNHLGNVKYLVIVFLIFFDLFLVNGRDVQNNIVDEIKYREEVCNDEV |
| | DLYLLMDCSGSIRRHNWVKHAVPLAMKLIQQLNLNENAIHLYVNIFS |
| | NNAKEIIRLHSDASKNKEKALIIIKSLLSTNLPYGRTNLSDALLQVRKH |
| | LNDRINRENANQLVVILTDGIPDSIQDSLKESRKLNDRGVKIAVFGIGQ |
| | GINVAFNRFLVGCHPSDGKCNLYADSAWENVKNVIGPFMKAVCVEV |
| | EKTASCGVWDEWSPCSVTCGKGTRSRKREILHEGCTSELQEQCEEER |
| | CPPKREPLDVPDEPEDDQPRPRGDNFAVEKPEENIIDNNPQEPSPNPEE |
| | GKGENPNGFDLDENPENPPNPDIPEQEPNIPEDSEKEVPSDVPKNPEDD |
| | REENFDIPKKPENKHDNQNNLPNDKSDRSIPYSPLPPKVLDNERKQSD |
| | PQSQDNNGNRHVPNSEDRETRPHGRNNENRSYNRKYNDTPKHPEREE |
| | HEKPDNNKKKGGSDNKYKIAGGIAGGLALLACAGLAYKFVVPGAAT |
| | PYAGEPAPFDETLGEEDKDLDEPEQFRLPEENEWN (SEQ ID NO: 42) |
| X13022 | MNHLGNVKYLVIVFLIFFDLFLVNGRDVQNNIVDEIKYSEEVCNDQV |
| | DLYLLMDCSGSIRRHNWVNHAVPLAMKLIQQLNLNDNAIHLYVNVF |
| | SNNAKEIIRLHSDASKNKEKALIIIRSLLSTNLPYGRTNLTDALLQVRK |
| | HLNDRINRENANQLVVILTDGIPDSIQDSLKESRKLSDRGVKIAVFGIG |
| | QGINVAFNRFLVGCHPSDGKCNLYADSAWENVKNVIGPFMKAVCVE |
| | VEKTASCGVWDEWSPCSVTCGKGTRSRKREILHEGCTSEIQEQCEEER |
| | CPPKWEPLDVPDEPEDDQPRPRGDNSSVQKPEENIIDNNPQEPSPNPEE |
| | GKDENPNGFDLDENPENPPNPDIPEQKPNIPEDSEKEVPSDVPKNPEDD |
| | REENFDIPKKPENKHDNQNNLPNDKSDRNIPYSPLPPKVLDNERKQSD |
| | PQSQDNNGNRHVPNSEDRETRPHGRNNENRSYNRKYNDTPKHPEREE |
| | HEKPDNNKKKGESDNKYKIAGGIAGGLALLACAGLAYKFVVPGAAT |
| | PYAGEPAPFDETLGEEDKDLDEPEQFRLPEENEWN (SEQ ID NO: 43) |
| AB807836 | MNHLGNVKYLVIVFLIFFDLFLVNGRDVQNNIVDEIKYREEVCNDEV |
| | DLYLLMDCSGSIRRHNWVKHAVPLAMKLIQQLNLNESAIHLYVNIFS |
| | NNAREIIRLHSDASKNKEKALIIIKSLLSTNLPYGRTNLTDALLQVRKH |
| | LNDRINRENASQLVVILTDGIPDSIQDSLKESRKLNDLGVKIAVFGIGQ |
| | GINVAFNRFLVGCHPSDGKCNLYADSAWENVKNVIGPFMKAVCVEV |
| | EKTASCGVWDEWSPCSVTCGKGTRSRKREILHEGCTSELQEQCEEER |
| | CPPKREPLDVPHEPEDDQPRPRGDNFAVEKPKENIIDNNPQEPSPNPEE |
| | GKGENPNGFDLDENPENPPNPDIPEQEPNIPEDSEKEVPSDVPKNPEDD |
| | REENFDIPKKPENKHDNQNNLPNDKSDRSIPYSPLPPKVLDNERKQSD |
| | PQSQDNNGNRHVPNSEDRETRPHGRNNENRSYNRKYNDTPKHPEREE |
| | HEKPDNNKKKGGSDNKYKIAGGIAGGLALLACAGLAYKFVVPGAAT |
| | PYAGEPAPFDETLGEEDKDLDEPEQFRLPEENEWN (SEQ ID NO: 44) |
| AB807839 | MNHLGNVKYLVIVFLIFFDLFLVNGRDVQNNIVDEIKYREEVCNDEV |
| | DLYLLMDCSGSIRRHNWVNHAVPLAMKLIQQLNLNESAIHLYLNIFS |
| | NNAREIIRLHSDASKNKEKALIIIKSLLNTNLPYGRTNLTDALLQVRKH |
| | LNDRINRENANQLVVILTDGIPDSIQDSLKESRKLNDRGVKIAVFGIGQ |
| | GINVAFNRFLVGCHPSDGKCNLYADSAWENVKNVIGPFMKAVCVEV |
| | EKTASCGVWDEWSPCSVTCGKGTRSRKREILHEGCTSELQEQCEEER |
| | CPPKREPLDVPHEPEDDQPRPRGDNFVVEKPEENIIDNNPQEPSPNPEE |
| | GKGENPNGFDLDENPENPPNPPNPPNPPNPDIPEQEPNIPEDSEKEVPSD |
| | VPKNPEDDREENFDIPKKPENKHDNQNNLPNDKSDRYIPYSPLPPKVL |
| | DNERKQSDPQSQDNNGNRHVPNSEDRETRPHGRNNENRSYNRKHNN |
| | TPKHPEREEHEKPDNNKKKGGSDNKYKIAGGIAGGLALLACAGLAY |
| | KFVVPGAATPYAGEPAPFDETLGEEDKDLDEPEQFRLPEENEWN |
| | (SEQ ID NO: 45) |

Sequences of other exogenous antigens

| | |
|---|---|
| IHNV | MDTMITTPLILILITCGANSQTVKPDTASESDQPTWSNPLFTYPEGCTL |
| Glycoprotein | DKLSKVNASQLRCPRIFDDENRGLIAYPTSIRSLSVGNDLGEIHTQGNH |
| | IHKVLYRTICSTGFFGGQTIEKALVEMKLSTKEAGAYDTTTAAALYFP |
| | APRCQWYTDNVQNDLIFYYTTQKSVLRDPYTRDFLDSDFIGGKCTKS |
| | PCQTHWSNVVWMGDAGIPACDSSQEIKGHLFVDKISNRVVKATSYG |
| | HHPWGLHQACMIEFCGQQWIRTDLGDLISVVYNSGSEILSFPKCEDKT |
| | VGMRGNLDDFAYLDDLVKASESREECLEAHAEIISTNSVTPYLLSKFR |
| | SPHPGINDVYAMHKGSIYHGMCMTVAVDEVSKDRTTYRAHRATSFT |
| | KWERPFGDEWEGFHGLHGNNTTIIPDLEKYVAQYKMSMMEPMSIKS |
| | VPHPSILALYNETDVSGISIRKLDSFDLQSLHWSFWPTISALGGIPFVLL |
| | LAVAACCCWSGRPPTPSVPQSIPMYHLANRS (SEQ ID NO: 68) |
| Canine | MSDGAVQPDGGQPAVRNERATGSGNGSGGGGGGGSGGVGISTGTFN |
| parvovirus | NQTEFKFLENGWVEITANSSRLVHLNMPESENYRRVVVNNLDKTAV |
| VP2 capsid | NGNMALDDTHAQIVTPWSLVDANAWGVWFNPGDWQLIVNTMSELH |
| | LVSFEQEIFNVVLKTVSESATQPPTKVYNNDLTASLMVALDSNNTMPF |
| | TPAAMRSETLGFYPWKPTIPTPWRYYFQWDRTLIPSHTGTSGTPTNIY |
| | HGTDPDDVQFYTIENSVPVHLLRTGDEFATGTFFFDCKPCRLTHTWQT |
| | NRALGLPPFLNSLPQAEGGTNFGYIGVQQDKRRGVTQMGNTNIITEAT |
| | IMRPAEVGYSAPYYSFEASTQGPFKTPIAAGRGGAQTDENQAADGDP |
| | RYAFGRQHGQKTTTTGETPERFTYIAHQDTGRYPEGDWIQNINFNLPV |
| | TNDNVLLPTDPIGGKTGINYTNIFNTYGPLTALNNVPPVYPNGQIWDK |
| | EFDTDLKPRLHVNAPFVCQNNCPGQLFVKVAPNLTNEYDPDASANM |
| | SRIVTYSDFWWKGKLVFKAKLRASHTWNPIQQMSINVDNQFNYVPS |
| | NIGGMKIVYEKSQLAPRKLY (SEQ ID NO: 69) |
| HIV envelope | MRVRGMQRNWQHLGKWGFLFLGILIICNAEDNLWVTVYYGVPVWK |
| glycoprotein | EATTTLFCASDAKGYEREVHNVWATHACVPTDPSPQEMVLENVTEN |
| GP41 | FNMWKNEMVEQMHTDIISLWDQSLKPCVKLTPLCVTLNCTDVDTNR |
| | TQNDNMTEERGVLKNCSFNMTTEVKDKRLKVSALFYRLDVVPISNNS |
| | NSSEYRLINCNTSTIKQACPKVSWDPIPIHYCAPAGYAILQCRDKQFNG |
| | TGPCKNVSTVQCTHGIKPVVSTQLLLNGSLAEKDIIIRCQNISDNTKTII |
| | VHLNESVQINCTRPNNNVVESIHLGPGQAFYATRRITGNIRKAYCNIN |
| | GTQWNNTLERVKTKLKTYFNKTITFNSASGGDLEVTMHSFNCRGEFF |
| | YCNTSELFKNAITPNVTIILQCRIKQIINMWQGVGQAMYASPIAGSITC |
| | NSSITGLLLTRDGGNDNVSTEEIFRPGGGNMKDNWRSELYKYKVVKI |
| | EPLGVAPTRAKRQVVKRDKRAVGLGAVFLGFLGAAGSTMGAASITL |
| | MVQARQLLSGIVQQQNNLLRAIEAQQHMLQLTVWGIKQLQARLLAV |
| | ERYLKDQQLLGIWGCSGKLICTTNVPWNSSWSNKSQEEIWGNMTWM |
| | EWEKEISNYSSTIYSLIEQSQNQQEKNEQELLALDKWTSLWNWFDISN |
| | WLWYIKIFIMIVGGLIGLRIVFAVLSIVNRVRKGYSPLSLQTLIPSPRGP |
| | DRPEGIEEGGGEQNRDRSVRLVNGFLALVWDDLRNLCLFSYRHLRDF |
| | ILIAARTVDRGLRRGWEALKYLWNLIQYWSRELKNSTTSLLDTTAVV |
| | VAEGTDRVIEALQRAGRAVLNVPRRIRQGAERALL (SEQ ID NO: 70) |

In some embodiments, oral antigenic compositions comprise a recombinant Spirulina, wherein the recombinant Spirulina a comprises at least one exogenous antigenic epitope having a sequence selected from the group consisting of: NANP (SEQ ID NO: 6), NVDP (SEQ ID NO: 7), NPDP (SEQ ID NO: 8), and a combination thereof. In some embodiments, multiple copies of these epitopes can be present in the recombinant Spirulina without being linked to any other protein; or as a part of a circumsporozoite protein containing these epitopes; or in the form of a fusion protein comprising one or more of these epitopes. Multiple copies of these epitopes can be present in the recombinant Spirulina in a variety of arrangement patterns, e.g., tandem and/or separated by spacer sequences, as described herein.

In some embodiments, oral antigenic compositions comprise a recombinant Spirulina comprising at least one antigenic epitope from a tumor antigen. The term “tumor antigen” as used herein refers to an antigen expressed on a cancer cell. In some embodiments, the recombinant Spirulina comprises at least one antigenic epitope from a tumor antigen expressed on a cancer cell including but not limited to, breast cancer cell, colon cancer cell, brain cancer cell, pancreatic cancer cell, lung cancer cell, cervical cancer cell, uterine cancer cell, prostate cancer cell, ovarian cancer cell, melanoma cancer cell, lymphoma cancer cell, myeloma cancer cell, and/or leukemic cancer cell.

In some embodiments, oral antigenic compositions comprise a recombinant Spirulina comprising at least one antigenic epitope from a self-antigen. The term “self-antigen” as used herein refers to an antigen associated with an autoimmune disease. In some embodiments, the recombinant Spirulina comprises at least one antigenic epitope from a self-antigen associated with an autoimmune disease including but not limited to, ulcerative colitis, rheumatoid arthritis, systemic lupus erythematosus (SLE), celiac disease, inflammatory bowel disease, Hashimoto's disease, Addison's disease, Grave's disease, type I diabetes, autoimmune thrombocytopenia purpura (ATP), idiopathic pulmonary fibrosis, idiopathic thrombocytopenia purpura (ITP), Crohn's disease, multiple sclerosis, and myasthenia gravis.

Oral antigenic compositions of the present disclosure comprise recombinant Spirulina in a non-living form. These non-living Spirulina containing an expressed exogenous antigen or epitope are then administered to a subject to elicit an immune response in the subject. In some embodiments, non-living recombinant Spirulina comprising at least one exogenous antigen or at least one exogenous antigenic epitope is prepared by drying the live culture of the recombinant Spirulina. Methods of drying include heat drying, e.g., drying in an oven; air-drying, spray drying, lyophilizing, or freeze-drying. Accordingly, in some embodiments, oral antigenic compositions of the present disclosure comprise a dried biomass of a recombinant Spirulina comprising at least one exogenous antigen or at least one exogenous antigenic epitope as described herein.

As used herein “Spirulina” is synonymous with “Arthrospira.” Oral antigenic compositions of the present disclosure can comprise any one of the following species of Spirulina: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jennerif, purpurea. A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor.

In some embodiments, oral antigenic compositions of the present disclosure can comprise one or more pharmaceutically acceptable excipients. Pharmaceutically acceptable carriers include but are not limited to saline, buffered saline, dextrose, water, glycerol, sterile isotonic aqueous buffer, and combinations thereof. In some embodiments, a pharmaceutically acceptable excipient is sodium bicarbonate.

In some embodiments, oral antigenic compositions of the present disclosure may comprise an adjuvant. As known in the art, the immunogenicity of a particular composition can be enhanced by the use of non-specific stimulators of the immune response, known as adjuvants. Exemplary adjuvants include a water-in-oil (W/O) emulsion composed of a mineral oil and a surfactant from the mannide monooleate family (e.g. MONTANIDE™ class of adjuvants) and flagellin adjuvants.

In some embodiments, oral antigenic compositions of the present disclosure comprise about 0.1% to about 5% of the total Spirulina biomass. In some embodiments, oral antigenic compositions of the present disclosure comprise about 1 mg to about 50 mg of the exogenous antigenic epitope per gram of dried Spirulina biomass. In some embodiments, oral antigenic compositions of the present disclosure comprise at least about 1 mg, 5 mg, 10 mg, 25 mg, 50mg, 100 mg, 200 mg, 300 mg, 500 mg, 750 mg, 1 mg, 5 mg, 10 mg, or 50 of the exogenous antigenic epitope per gram of dried Spirulina biomass.

### Uses of Oral Antigenic Compositions

Oral antigenic compositions of the present disclosure can be used as a vaccine. In some embodiments, oral antigenic compositions can be used to induce an immune response in a subject. For example, oral antigenic compositions can be used to induce an immune response directed to an infectious microorganism, a tumor antigen, or a self-antigen. In some embodiments, oral antigenic compositions can be used to reduce the severity of an infection in a subject in need thereof. In some embodiments, oral antigenic compositions can be used to prevent infection in a subject. In some embodiments, oral antigenic compositions can be used to prevent disease in a subject. In some embodiments, oral antigenic compositions can be used to reduce the severity of a disease in a subject. In some embodiments, oral antigenic compositions can be used to prevent or delay recurrence of a disease in a subject. In some embodiments, oral antigenic compositions can be used to prevent or delay recurrence of a cancer in a subject.

In some embodiments, provided herein are methods of inducing an immune response in a subject in need thereof comprising administering to the subject any of the oral antigenic compositions described herein. Without wishing to be bound to a theory, it is expected that when the oral antigenic composition of the present disclosure is administered to a subject, the at least one exogenous antigenic epitope is recognized by immune cells of the subject, such as T cells or B cells, thereby activating an immune response against the exogenous antigenic epitope. In some embodiments, administration of oral antigenic compositions described herein can induce a humoral immune response and/or a cellular immune response.

Oral antigenic compositions of the present disclosure can be administered according to a schedule, for example, administering a priming dose of the antigenic composition and subsequently administering one or more booster doses of the antigenic composition. In some embodiments, a first booster dose of the antigenic composition can be administered anywhere from about two weeks to about 10 years after the priming dose. In some embodiments, a first booster dose of the antigenic composition can be administered anywhere from about two weeks, 1 month, 2 months, 3 months, 4 months, 6 months, 9 months, 1 year, 2 years, 3 years, or 5 years after the priming dose. A second booster dose of the antigenic composition can be administered after the first booster dose and anywhere from about 3 months to about 10 years after the priming dose. In some embodiments, a second booster dose of the antigenic composition can be administered after the first booster dose and from about 3 months, 4 months, 6 months, 9 months, 1 year, 2 years, 3 years, or 5 years after the priming dose. The third booster dose may be optionally administered when no or low levels of specific immunoglobulins are detected in the serum and/or other bodily fluids of the subject after the second booster dose.

In some embodiments, antigenic compositions other than the oral antigenic compositions of the present disclosure can be administered prior to the administration of the present compositions to prime the subject's immune response. In these embodiments, methods of the present disclosure comprise administering an antigenic composition other than the present oral antigenic composition as a priming dose and subsequently administering one or more booster doses of the present oral antigenic composition.

Oral antigenic compositions of the present disclosure can be used to induce an immune response to and/or prevent or reduce the severity of a disease or an infection caused by a virus, bacterium, parasite, or fungus.

In some embodiments, oral antigenic compositions can be used as a vaccine for, or to induce an immune response to and/or reduce the severity of malaria.

In some embodiments, oral antigenic compositions can be used as a vaccine for, or to induce an immune response to and/or reduce the severity of an infection such as tetanus, diphtheria, pertussis, pneumonia, meningitis, campylobacteriosis, mumps, measles, rubella, polio, flu, hepatitis, chickenpox, malaria, toxoplasmosis, giardiasis, or leishmaniasis.

In some embodiments, oral antigenic compositions described herein can be used to induce an immune response to and/or reduce the severity of an infection caused by a virus including, but not limited to, bacteriophage, RNA bacteriophage (e.g. MS2, AP205, PP7 and Qβ), Helicobacter pylori, infectious haematopoietic necrosis virus (IHNV), parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, Poliovirus, Norovirus, Zika Virus, Denge Virus, Rabies Virus, Newcastle Disease Virus, and White Spot Syndrome Virus.

In some embodiments, oral antigenic compositions described herein can be used to induce an immune response to and/or reduce the severity of an infection caused by IHNV.

In some embodiments, oral antigenic compositions described herein can be used to induce an immune response to and/or reduce the severity of an infection caused by a parvovirus, e.g., canine parvovirus.

In some embodiments, oral antigenic compositions described herein can be used to induce an immune response to and/or reduce the severity of an infection caused by a bacterium including, but not limited to, Mycobacterium, Streptococcus, Staphylococcus, Shigella, Campylobacter, Salmonella, Clostridium, Corynebacterium, Pseudomonas, Neisseria, Listeria, Vibrio, Bordetella, and Legionella.

In some embodiments, oral antigenic compositions described herein can be used to induce an immune response to and/or reduce the severity of an infection caused by a parasite including, but not limited to, Plasmodium, Trypanosoma, Toxoplasma, Giardia, and Leishmania, Cryptosporidium, helminthic parasites: Trichuris spp. (whipworms), Enterobius spp. (pinworms), Ascaris spp. (roundworms), Ancylostoma spp. and Necator spp. (hookworms), Strongyloides spp. (threadworms), Dracunculus spp. (Guinea worms), Onchocerca spp. and Wuchereria spp. (filarial worms), Taenia spp., Echinococcus spp., and Diphyllobothrium spp. (human and animal cestodes), Fasciola spp. (liver flukes) and Schistosoma spp. (blood flukes).

In some embodiments, oral antigenic compositions described herein can be used to induce an immune response to and/or reduce the severity of an infection caused by Plasmodium. In some embodiments, oral antigenic compositions of the present disclosure can be used to induce an immune response to and/or reduce the severity of an infection caused by a Plasmodium selected from the group consisting of: P. falciparum, P. malariae, P. ovale and P. vivax.

In some embodiments, oral antigenic compositions described herein can be used to induce an immune response to and/or reduce the severity of an infection caused by a fungus including but not limited to Aspergillus, Candida, Blastomyces, Coccidioides, Cryptococcus, and Histoplasma. In some embodiments, oral antigenic compositions can be used to induce an immune response to and/or reduce the severity of a Candida albicans or a Candida auris infection.

In some embodiments, oral antigenic compositions described herein can be used to induce an immune response to a tumor antigen. In some embodiments, the oral antigenic compositions can be used to induce an immune response to a tumor antigen expressed on a cancer cell including but not limited to breast cancer cell, colon cancer cell, brain cancer cell, pancreatic cancer cell, lung cancer cell, cervical cancer cell, uterine cancer cell, prostate cancer cell, ovarian cancer cell, melanoma cancer cell, lymphoma cancer cell, myeloma cancer cell, and leukemic cancer cell.

In some embodiments, oral antigenic compositions described herein can be used to induce an immune response to a self-antigen. In some embodiments, the oral antigenic compositions can be used to induce an immune response to a self-antigen associated with an autoimmune disease including but not limited to ulcerative colitis, rheumatoid arthritis, systemic lupus erythematosus (SLE), celiac disease, inflammatory bowel disease, Hashimoto's disease, Addison's disease, Grave's disease, type I diabetes, autoimmune thrombocytopeni purpura (ATP), idiopathic pulmonary fibrosis, idiopathic thrombocytopenia purpura (ITP), Crohn's disease, multiple sclerosis, and myasthenia gravis.

It is understood that antigenic compositions of the present disclosure are administered orally.

The dosage of the oral antigenic composition can be determined readily by the skilled artisan, for example, by first identifying doses effective to elicit a prophylactic or therapeutic immune response, e.g., by measuring the serum titer of specific immunoglobulins or by measuring the inhibitory ratio of antibodies in serum samples, or bodily fluid samples. Said dosages can be determined from animal studies. A non-limiting list of animals used to study the efficacy of vaccines include the guinea pig, hamster, ferrets, chinchilla, mouse and cotton rat. Study animals may not be the natural hosts to infectious agents but can still serve in studies of various aspects of the disease. For example, any of the above animals can be dosed with an oral antigenic composition of the present disclosure, e.g. a recombinant Spirulina comprising a VLP comprising Plasmodium antigen/antigenic epitope, to partially characterize the immune response induced, and/or to determine if any neutralizing antibodies have been produced.

In addition, human clinical studies can be performed to determine the preferred effective dose for humans by a skilled artisan. Such clinical studies are routine and well known in the art. Effective doses may be extrapolated from dose-response curves derived from in vitro studies, animal studies, and/or clinical studies.

### Methods of Making Oral Antigenic Compositions

Provided are methods of making oral antigenic compositions described herein. Methods of making oral antigenic compositions comprise introducing into a Spirulina a a nucleic acid sequence encoding the at least one exogenous antigenic epitope. In some embodiments, the nucleic acid sequence encodes for an exogenous antigen comprising the at least one exogenous antigenic epitope. In some embodiments, the nucleic acid sequence encodes for a fusion protein comprising the at least one exogenous antigenic epitope.

In some embodiments, methods of making oral antigenic compositions comprise introducing into a Spirulina a nucleic acid sequence comprising a sequence selected from Table 4.

TABLE 4

| Description of the | |
|---|---|
| fusion protein | Sequence |
| P. yoelii CSP B cell | atggacatagatccctataaagaatttggttcatcttatcagttgttgaattttcttcctttggacttctt |
| epitope in monomer | tcctgaccttaatgctttggtggacactgctactgccttgtatgaagaagagctaacaggtaggg |
| WHcAg | aacattgctctccgcaccatacagctattagacaagctttagtatgctgggatgaattaactaaatt |
| | gatagcttggatgagctctaacataacttctCAGGGTCCAGGTGCTCCACAA |
| | GGACCTGGAGCACCTCAGGGCCCTGGCGCTCCCCAAGGG |
| | CCTGGAGCCCCTCAGGGACCCGGTGCACCGCAAGGTCCG |
| | GGCGCTCCCCAAGAACCACCTCAACAGCCTCCACAGCAA |
| | CCTCCTCAGCAGCCACCACAACAGCCCCCTCAGCAAgaaca |
| | agtaagaacaatcatagtaaatcatgtcaatgatacctggggacttaaggtgagacaaagtttat |
| | ggtttcatttgtcatgtctcacttttggacaacatacagttcaagaatttttagtaagttttggagtatg |
| | gatcagaactccagctccatatagacctcctaatgcacccattctctcgactcttccggaacatac |
| | agtcattAATGAAGATAGTTATGTTCCTTCTGCTGAACAAATT |
| | TTAGAATTTGAGCAAAAATTAATTAGCGAGGAAGACCTA |
| | GAACAAAAACTGATCTCTGAAGAGGATCTGTAA (SEQ ID |
| | NO: 46) |
| P. falciparum CSP | ATGGACATAGATCCCTATAAAGAATTTGGTTCATCTTATC |
| B cell epitope in | AGTTGTTGAATTTTCTACCTTTGGACTTCTTTCCTGACCTA |
| tandem WHcAg | AATGCTTTGGTGGACACTGCTACTGCCTTGTATGAAGAAG |
| dimer | AGCTAACAGGTCGAGAACATTGCTCTCCGCACCATACAG |
| | CTATTAGACAAGCTTTAGTATGCTGGGATGAATTAACTAA |
| | ATTGATAGCTTGGATGAGCTCTAACATAACTTCTGAACAA |
| | GTAAGAACAATCATAGTAAATCATGTCAATGATACCTGG |
| | GGATTAAAGGTGAGACAAAGTTTATGGTTTCATTTGTCAT |
| | GTTTGACTTTTGGACAACATACAGTTCAAGAATTTTTAGT |
| | AAGTTTTGGAGTATGGATCAGAACTCCAGCTCCATATAG |
| | ACCTCCTAATGCACCCATTCTCTCCACTCTTCCCGAACAT |
| | ACAGTCATTGGTGGAAGTGGAGGGTCTGGTGGGTCCGGG |
| | GGTAGTGGTGGGTCTGATATCGATCCCTACAAAGAATTC |
| | GGCAGTTCTTATCAGTTACTAAATTTCCTGCCGCTGGATT |
| | TTTTTCCCGATCTGAACGCCTTGGTCGATACTGCCACCGC |
| | CTTGTACGAGGAAGAGCTAACCGGGCGAGAGCATTGTAG |
| | TCCACATCATACTGCTATCCGCCAGGCTCTGGTCTGCTGG |
| | GACGAATTGACCAAGTTAATTGCATGGATGAGCTCCAAT |
| | ATTACTAGTgaagagggtAAtGCaAAcCCtAATGCgAACCCgAAc |
| | GCtAACCCtAAtGCcAAtCCtAACGCTAAtCCcAAtGCcAAcCCg |
| | AATGCaAAcCCaAAtGCgAATCCgAATGCtAAcCCgAAcGCtA |
| | AcCCgAATGCgAATCCaAACGCgAAcCCcAAcGCaAATCCgA |
| | AtGCaAACCCtAATGCaAAtCCaggtgaagagGAGCAGGTCCGCA |
| | CGATCATTGTTAACCACGTCAACGATACCTGGGGCCTAA |
| | AGGTTCGCCAATCTTTGTGGTTCCATCTGTCGTGCCTGAC |
| | CTTTGGGCAACACACCGTCCAGGAGTTCCTGGTGAGCTTC |
| | GGCGTTTGGATCCGCACCCCAGCACCCTACCGCCCGCCA |
| | AATGCTCCCATTTTAAGTACCTTGCCCGAACACACCGTGA |
| | TTCACCACCATCATCACCACTAA (SEQ ID NO: 47) |
| P. falciparum CSP | ATGGACATAGATCCCTATAAAGAATTTGGTTCATCTTATC |
| B cell epitope + | AGTTGTTGAATTTTCTACCTTTGGACTTCTTTCCTGACCTA |
| Salmonella fliC | AATGCTTTGGTGGACACTGCTACTGCCTTGTATGAAGAAG |
| sequences in tandem | AGCTAACAGGTCGAGAACATTGCTCTCCGCACCATACAG |
| WHcAg dimer | CTATTAGACAAGCTTTAGTATGCTGGGATGAATTAACTAA |
| | ATTGATAGCTTGGATGAGCTCTAACATAACTTCTGAACAA |
| | GTAAGAACAATCATAGTAAATCATGTCAATGATACCTGG |
| | GGATTAAAGGTGAGACAAAGTTTATGGTTTCATTTGTCAT |
| | GTTTGACTTTTGGACAACATACAGTTCAAGAATTTTTAGT |
| | AAGTTTTGGAGTATGGATCAGAACTCCAGCTCCATATAG |
| | ACCTCCTAATGCACCCATTCTCTCCACTCTTCCCGAACAT |
| | ACAGTCATTGGTGGAAGTGGAGGGTCTGGTGGGTCCGGG |
| | GGTAGTGGTGGGTCTGATATCGATCCCTACAAAGAATTC |
| | GGCAGTTCTTATCAGTTACTAAATTTCCTGCCGCTGGATT |
| | TTTTTCCCGATCTGAACGCCTTGGTCGATACTGCCACCGC |
| | CTTGTACGAGGAAGAGCTAACCGGGCGAGAGCATTGTAG |
| | TCCACATCATACTGCTATCCGCCAGGCTCTGGTCTGCTGG |
| | GACGAATTGACCAAGTTAATTGCATGGATGAGCTCCAAT |
| | ATTACTAGTgaagagggtGCGCAGGTCATTAACACTAATTCGC |
| | TGAGTTTACTAACACAGAATAATCTAAACAAGAGCCAAT |
| | CCGCCCTTGGCACGGCGATCGAGCGGCTGAGTTCGGGGC |
| | TGCGTATCAATAGTGCCAAAGATGACGCGGCCGGCCAGG |
| | CGATTGCAAATCGTTTTACGGCCAATATAAAGGGGCTTAC |
| | ACAGGCTTCTCGTAATGCCAACGACGGTATTTCCATTGCC |
| | CAAACAACGGAAGGCGCGCTGAATGAAATCAATAATAAT |
| | CTGCAGCGGGTCCGGGAGTTAGCGGTGCAGTCTGCCAAC |
| | TCAACAAATTCTCAATCAGATCTGGATTCTATCCAGGCAG |
| | AAATAACTCAGAGGCTTAATGAAATCGATCGTGTTTCTGG |
| | ACAAACCCAGTTTAATGGTGTCAAGGTCCTTGCTCAGGAC |
| | AACACCCTGACCATCCAGGTAGGCGCGAACGATGGAGAA |
| | ACCATTGATATTGATCTGAAACAGATTAATTCTCAGACTC |
| | TAGGTCTTGACACCTTGAATGTGCAGGGTTCTAAtGCaAAc |
| | CCtAATGCgAACCCgAAcGCtAACCCtAAtGCcAAtCCtAACGC |
| | TAAtCCcAAtGCcAAcCCgAATGCaAAcCCaAAtGCgAATCCgA |
| | ATGCtAAcCCgAAcGCtAAcCCgAATGCgAATCCaAACGCgAA |
| | CCCcAAcGCaAATCCgAAtGCaAACCCtAATGCaAAtCCaGGTT |
| | CTACCACAACCGAGAATCCTCTGCAGAAAATCGATGCTG |
| | CTCTCGCGCAAGTGGACACTTTGCGTTCAGATTTGGGAGC |
| | TGTGCAAAATCGTTTCAACAGCGCGATTACAAACCTGGG |
| | TAACACCGTAAACAATCTGACTAGTGCCCGGAGTCGGAT |
| | TGAAGATAGCGATTATGCGACCGAAGTGTCTAACATGAG |
| | CCGGGCCCAAATCTTGCAGCAAGCCGGCACTAGTGTTCT |
| | GGCGCAAGCAAATCAGGTCCCCCAAAACGTTCTCAGCCT |
| | TCTGCGGggtgaagagGAGCAGGTCCGCACGATCATTGTTAA |
| | CCACGTCAACGATACCTGGGGCCTAAAGGTTCGCCAATC |
| | TTTGTGGTTCCATCTGTCGTGCCTGACCTTTGGGCAACAC |
| | ACCGTCCAGGAGTTCCTGGTGAGCTTCGGCGTTTGGATCC |
| | GCACCCCAGCACCCTACCGCCCGCCAAATGCTCCCATTTT |
| | AAGTACCTTGCCCGAACACACCGTGATTCACCACCATCAT |
| | CACCACTAA (SEQ ID NO: 48) |
| Canine parvovirus | ATGGACATAGATCCCTATAAAGAATTTGGTTCATCTTATC |
| 2L21 peptide | AGTTGTTGAATTTTCTACCTTTGGACTTCTTTCCTGACCTA |
| epitope in 2x | AATGCTTTGGTGGACACTGCTACTGCCTTGTATGAAGAAG |
| configuration; | AGCTAACAGGTCGAGAACATTGCTCTCCGCACCATACAG |
| monomer WhcAg | CTATTAGACAAGCTTTAGTATGCTGGGATGAATTAACTAA |
| | ATTGATAGCTTGGATGAGCTCTAACATAACTTCTgaagagggt |
| | TCTGACGGTGCTGTGCAGCCCGATGGCGGTCAACCCGCT |
| | GTTCGTAATGAACGTGCTACTGGTGGTGGCTCTAGTGATG |
| | GTGCTGTTCAGCCTGACGGTGGTCAACCTGCTGTGCGCAA |
| | CGAGCGTGCAACAGGAggtgaagagGAACAAGTAAGAACAA |
| | TCATAGTAAATCATGTCAATGATACCTGGGGATTAAAGG |
| | TGAGACAAAGTTTATGGTTTCATTTGTCATGTTTGACTTTT |
| | GGACAACATACAGTTCAAGAATTTTTAGTAAGTTTTGGAG |
| | TATGGATCAGAACTCCAGCTCCATATAGACCTCCTAATGC |
| | ACCCATTCTCTCCACTCTTCCCGAACATACAGTCATTCAC |
| | CACCATCATCACCACTAA (SEQ ID NO: 49) |
| Canine parvovirus | ATGGACATAGATCCCTATAAAGAATTTGGTTCATCTTATC |
| 3L17 peptide | AGTTGTTGAATTTTCTACCTTTGGACTTCTTTCCTGACCTA |
| epitope in 4x | AATGCTTTGGTGGACACTGCTACTGCCTTGTATGAAGAAG |
| configuration; | AGCTAACAGGTCGAGAACATTGCTCTCCGCACCATACAG |
| monomer WhcAg | CTATTAGACAAGCTTTAGTATGCTGGGATGAATTAACTAA |
| | ATTGATAGCTTGGATGAGCTCTAACATAACTTCTgaagagggt |
| | GACGGTGCTGTGCAGCCCGATGGCGGTCAACCCGCTGTT |
| | CGTAATGAACGTGGTGGCTCTGATGGTGCTGTTCAGCCTG |
| | ACGGTGGTCAACCTGCTGTGCGCAACGAGCGTGGTGGTT |
| | CCGACGGTGCCGTTCAACCCGACGGTGGCCAACCCGCCG |
| | TGCGTAATGAGCGCGGTGGTTCTGACGGCGCTGTGCAAC |
| | CTGACGGCGGTCAGCCCGCCGTTCGTAACGAGCGTggtgaag |
| | agGAACAAGTAAGAACAATCATAGTAAATCATGTCAATGA |
| | TACCTGGGGATTAAAGGTGAGACAAAGTTTATGGTTTCAT |
| | TTGTCATGTTTGACTTTTGGACAACATACAGTTCAAGAAT |
| | TTTTAGTAAGTTTTGGAGTATGGATCAGAACTCCAGCTCC |
| | ATATAGACCTCCTAATGCACCCATTCTCTCCACTCTTCCC |
| | GAACATACAGTCATTCACCACCATCATCACCACTAA (SEQ |
| | ID NO: 50) |
| IHNV vaccine; | ATGGACATAGATCCCTATAAAGAATTTGGTTCATCTTATC |
| E1 + E2 epitopes in | AGTTGTTGAATTTTCTACCTTTGGACTTCTTTCCTGACCTA |
| tandem WHcAg | AATGCTTTGGTGGACACTGCTACTGCCTTGTATGAAGAAG |
| dimer | AGCTAACAGGTCGAGAACATTGCTCTCCGCACCATACAG |
| | CTATTAGACAAGCTTTAGTATGCTGGGATGAATTAACTAA |
| | ATTGATAGCTTGGATGAGCTCTAACATAACTTCTGAACAA |
| | GTAAGAACAATCATAGTAAATCATGTCAATGATACCTGG |
| | GGATTAAAGGTGAGACAAAGTTTATGGTTTCATTTGTCAT |
| | GTTTGACTTTTGGACAACATACAGTTCAAGAATTTTTAGT |
| | AAGTTTTGGAGTATGGATCAGAACTCCAGCTCCATATAG |
| | ACCTCCTAATGCACCCATTCTCTCCACTCTTCCCGAACAT |
| | ACAGTCATTGGTGGAAGTGGAGGGTCTGGTGGGTCCGGG |
| | GGTAGTGGTGGGTCTGATATCGATCCCTACAAAGAATTC |
| | GGCAGTTCTTATCAGTTACTAAATTTCCTGCCGCTGGATT |
| | TTTTTCCCGATCTGAACGCCTTGGTCGATACTGCCACCGC |
| | CTTGTACGAGGAAGAGCTAACCGGGCGAGAGCATTGTAG |
| | TCCACATCATACTGCTATCCGCCAGGCTCTGGTCTGCTGG |
| | GACGAATTGACCAAGTTAATTGCATGGATGAGCTCCAAT |
| | ATTACTAGTCCTGGTGGAAGTGGAGACGATGAAAATCGT |
| | GGCTTGATCGCTTATCCTACCAGTATCCGTTCCTTGAGTG |
| | TCGGCGGAAGTGGAGGGTCTGATCTGATTAGCGTGGTTT |
| | ACAACAGTGGAAGCGAGATCCTGTCGTTTCCTGGTGGAT |
| | CAGGGGAGCAGGTCCGCACGATCATTGTTAACCACGTCA |
| | ACGATACCTGGGGCCTAAAGGTTCGCCAATCTTTGTGGTT |
| | CCATCTGTCGTGCCTGACCTTTGGGCAACACACCGTCCAG |
| | GAGTTCCTGGTGAGCTTCGGCGTTTGGATCCGCACCCCAG |
| | CACCCTACCGCCCGCCAAATGCTCCCATTTTAAGTACCTT |
| | GCCCGAACACACCGTGATTGAGCAAAAATTAATTAGCGA |
| | GGAAGACCTAGAACAAAAACTGATCTCTGAAGAGGATCT |
| | GTAA (SEQ ID NO: 51) |
| IHNV vaccine; DIII | ATGGACATAGATCCCTATAAAGAATTTGGTTCATCTTATC |
| epitope in tandem | AGTTGTTGAATTTTCTACCTTTGGACTTCTTTCCTGACCTA |
| WHcAg dimer | AATGCTTTGGTGGACACTGCTACTGCCTTGTATGAAGAAG |
| | AGCTAACAGGTCGAGAACATTGCTCTCCGCACCATACAG |
| | CTATTAGACAAGCTTTAGTATGCTGGGATGAATTAACTAA |
| | ATTGATAGCTTGGATGAGCTCTAACATAACTTCTGAACAA |
| | GTAAGAACAATCATAGTAAATCATGTCAATGATACCTGG |
| | GGATTAAAGGTGAGACAAAGTTTATGGTTTCATTTGTCAT |
| | GTTTGACTTTTGGACAACATACAGTTCAAGAATTTTTAGT |
| | AAGTTTTGGAGTATGGATCAGAACTCCAGCTCCATATAG |
| | ACCTCCTAATGCACCCATTCTCTCCACTCTTCCCGAACAT |
| | ACAGTCATTGGTGGAAGTGGAGGGTCTGGTGGGTCCGGG |
| | GGTAGTGGTGGGTCTGATATCGATCCCTACAAAGAATTC |
| | GGCAGTTCTTATCAGTTACTAAATTTCCTGCCGCTGGATT |
| | TTTTTCCCGATCTGAACGCCTTGGTCGATACTGCCACCGC |
| | CTTGTACGAGGAAGAGCTAACCGGGCGAGAGCATTGTAG |
| | TCCACATCATACTGCTATCCGCCAGGCTCTGGTCTGCTGG |
| | GACGAATTGACCAAGTTAATTGCATGGATGAGCTCCAAT |
| | ATTACTAGTCCTGGTGGAAGTGGAgacgatgagaacagggggctaatt |
| | gcctatcccacatccatccggtccctgtcagtcggaaacgacGGTGGCAGTGGAG |
| | GGTCTagccaagagataaaagctcacctctttgttgataaaatctccaatcgagtcgtgaag |
| | gcaacgagctacggacaccacccctggggactgcatcaggcctgtatgattgaattctgtggg |
| | caacagtggatacggacagatctcggtgacctaatatctgtcgtatacaattctggatcagaaat |
| | cctctcgttcccgaagtgtgaagacaagaccgtgggaCCAGCAGAGGGTGGCG |
| | GTCCAGCAGGTGGATCAGGGGAGCAGGTCCGCACGATCA |
| | TTGTTAACCACGTCAACGATACCTGGGGCCTAAAGGTTCG |
| | CCAATCTTTGTGGTTCCATCTGTCGTGCCTGACCTTTGGG |
| | CAACACACCGTCCAGGAGTTCCTGGTGAGCTTCGGCGTTT |
| | GGATCCGCACCCCAGCACCCTACCGCCCGCCAAATGCTC |
| | CCATTTTAAGTACCTTGCCCGAACACACCGTGATTGAGCA |
| | AAAATTAATTAGCGAGGAAGACCTAGAACAAAAACTGAT |
| | CTCTGAAGAGGATCTGTAA (SEQ ID NO: 52) |

Any appropriate means for transforming Spirulina may be used in the present disclosure. Exemplary methods for transforming Spirulina to express a heterologous protein are described in U.S. Pat. No. 10,131,870, which is incorporated by reference herein in its entirety.

In some embodiments, methods of making an oral antigenic composition comprising introducing an expression vector having a nucleic acid sequence encoding the at least one exogenous antigenic epitope into a Spirulina cell. In some embodiments, the vector is not integrated into the Spirulina genome. In some embodiments, the vector is a high copy or a high expression vector. In some embodiments the nucleic acid sequence encoding the at least one exogenous antigenic epitope is under the control of a strong promoter. In some embodiments the nucleic acid sequence encoding the at least one exogenous antigenic epitope is under the control of a constitutive promoter. In some embodiments the nucleic acid sequence encoding the at least one exogenous antigenic epitope is under the control of an inducible promoter.

In some embodiments, methods of making an oral antigenic composition comprise introducing a vector having homology arms and a nucleic acid sequence encoding the at least one exogenous antigenic epitope into a Spirulina cell. Upon homologous recombination, the nucleic acid sequence encoding the at least one exogenous antigenic epitope is integrated into the Spirulina genome.

In some embodiments, a vector having homology arms and a nucleic acid sequence encoding the at least one exogenous antigenic epitope can be introduced into Spirulina using electroporation. The electroporation is preferably carried out in the presence of an appropriate osmotic stabilizer.

Prior to introduction of the vector into Spirulina, Spirulina may be cultured in any suitable media for growth of cyanobacteria such as SOT medium. SOT medium includes NaHCO₃ 1.68 g, K₂HPO₄ 50 mg, NaNO₃ 250 mg, K₂50₄ 100 mg, NaCl 100 mg, MgSO₄·7H₂O, 20 mg, CaCl₂·2H₂O 4 mg, FeSO₄·7H₂O 1 mg, Na₂EDTA.2H₂O 8 mg, A₅ solution 0.1 mL, and distilled water 99.9 mL. A₅ solution includes H₃BO₃ 286 mg, MnSO₄·5H₂O) 217 mg, ZnSO₄·7H₂O 22.2 mg, CuSO₄·5H₂O 7.9 mg, Na₂MoO₄·2H₂O 2.1 mg, and distilled water 100 mL. Cultivation may occur with shaking (e.g., 100-300 rpm) at a temperature higher than room temperature (e.g. 25-37° C.) and under continuous illumination (e.g. 20-2,000, 50-500, or 100-200 μmol photon m⁻²s⁻¹). The growing cells may be harvested when the optical density at 750 nm reaches a predetermined threshold (e.g., OD₇₅₀ of 0.3-2.0, 0.5-1.0, or 0.6-0.8). A volume of the harvested cells may be concentrated by centrifugation then resuspended in a solution of pH balancer and salt. The pH balancer may be any suitable buffer that maintains viability of Spirulina while keeping pH of the media between 6 and 9 pH, between 6.5 and 8.5 pH, or between 7 and 8 pH. Suitable pH balancers include HEPES, HEPES-NaOH, sodium or potassium phosphate buffer, and TES. The salt solution may be NaCl at a concentration of between 50 mM and 500 mM, between 100 mM and 400 mM, or between 200 mM and 300 mM. In an embodiment between 1-50 mL of 1-100 mM pH balance may be used to neutralize the pH.

Cells collected by centrifugation may be washed with an osmotic stabilizer and optionally a salt solution (e.g. 1-50 mL of 0.1-100 mM NaCl). Any amount of the culture may be concentrated by centrifugation. In an embodiment between 5-500 mL of the culture may be centrifuged. The osmotic stabilizer may be any type of osmotic balancer that stabilizes cell integrity of Spriulina during electroporation. In an embodiment, the osmotic stabilizer may be a sugar (e.g. w/v 0.1-25%) such as glucose or sucrose. In an embodiment the osmotic stabilizer may be a simple polyol (e.g. v/v 1-25%) including glycerine, glycerin, or glycerol. In an embodiment the osmotic stabilizer may be a polyether including (e.g. w/v 0.1-20%) polyethylene glycol (PEG), poly(oxyethylene), or poly(ethylene oxide) (PEO). The PEG or PEO may have any molecular weight from 200 to 10,000, from 1000 to 6000, or from 2000 to 4000. In an embodiment the pH balancer or buffer may be used instead of or in addition to the osmotic stabilizer.

A vector having homology arms and a nucleic acid sequence encoding the at least one exogenous antigenic epitope can be introduced into Spirulina cells that are cultured and washed with an osmotic stabilizer as described above. Electroporation can be used to introduce the vector.

Electroporation may be performed in a 0.1-, 0.2- or 0.4-cm electroporation cuvette at between 0.6 and 10 kV/cm, between 2.5 and 6.5 kV/cm, or between 4.0 and 5.0 kV/cm; between 1 and 100 μF, between 30 and 70 μF, or between 45 and 55 μF; and between 10 and 500 mΩ, between 50 and 250 mΩ, or between 90 and 110 mΩ. In some embodiments, electroporation may be performed at 4.5 kV/cm, 50 μf, and 100 mΩ.

Following electroporation the cells may be grown in the presence of one or more antibiotics selected based on resistance conferred through successful transformation with the plasmid. Post-electroporation culturing may be performed at reduced illumination levels (e.g. 5-500, 10-100, or 30-60 μmol photon m⁻² s⁻¹). The culturing may also be performed with shaking (e.g. 100-300 rpm). The level of antibiotics in the media may be between 5 and 100 μg/mL. Post-electroporation culturing may be continued for 1-5 days or longer. Successful transformants identified by antibiotic resistance may be selected over a time course of 1 week to 1 month on plates or in 5-100 mL of SOT medium supplemented with 0.1-2.0 μg of appropriate antibiotics.

A vector used in the methods can be a plasmid, bacteriophage, or a viral vector into which a nucleic acid sequence encoding the at least one exogenous antigen can be inserted or cloned. A vector may comprise one or more specific sequences that allow recombination into a particular, desired site of the Spirulina's chromosome. These specific sequences may be homologous to sequences present in the wild-type Spirulina. A vector system can comprise a single vector or plasmid, two or more vectors or plasmids, some of which increase the efficiency of targeted mutagenesis, or a transposition. The choice of the vector will typically depend on the compatibility of the vector with the Spirulina cell into which the vector is to be introduced. The vector can include a reporter gene, such as a green fluorescent protein (GFP), which can be either fused in frame to one or more of the encoded antigenic epitopes, or expressed separately. The vector can also include a positive selection marker such as an antibiotic resistance gene that can be used for selection of suitable transformants. The vector can also include a negative selection marker such as the type II thioesterase (tesA) gene or the Bacillus subtilis structural gene (sacB). Use of a reporter or marker allows for identification of those cells that have been successfully transformed with the vector.

In some embodiments, the vector includes one or two homology arms that are homologous to DNA sequences of the Spirulina genome that are adjacent to the targeted locus. The sequence of the homology arms can be partially or fully complementary to the regions of Spirulina genome adjacent to the targeted locus.

The homology arms can be of any length that allows for site-specific homologous recombination. A homology arm may be any length between about 2000 bp and 500 bp. For example, a homology arm may be about 2000 bp, about 1500 bp, about 1000 bp, or about 500 bp. In some embodiments having two homology arms, the homology arms may be the same or different length. Thus, each of the two homology arms may be any length between about 2000 bp and 500 bp. For example, each of the two homology arms may be about 2000 bp, about 1500 bp. about 1000 bp, or about 500 bp.

A portion of the vector adjacent to one homology arm or flanked by two homology arms modifies the targeted locus in the Spirulina genome by homologous recombination. The modification may change a length of the targeted locus including a deletion of nucleotides or addition of nucleotides. The addition or deletion may be of any length. The modification may also change a sequence of the nucleotides in the targeted locus without changing the length. The targeted locus may be any portion of the Spirulina genome including coding regions, non-coding regions, and regulatory sequences.

### EXAMPLES

### Example 1: Spirulina Engineered to Express WHcAg VLPS With Plasmodium CSP Antigens

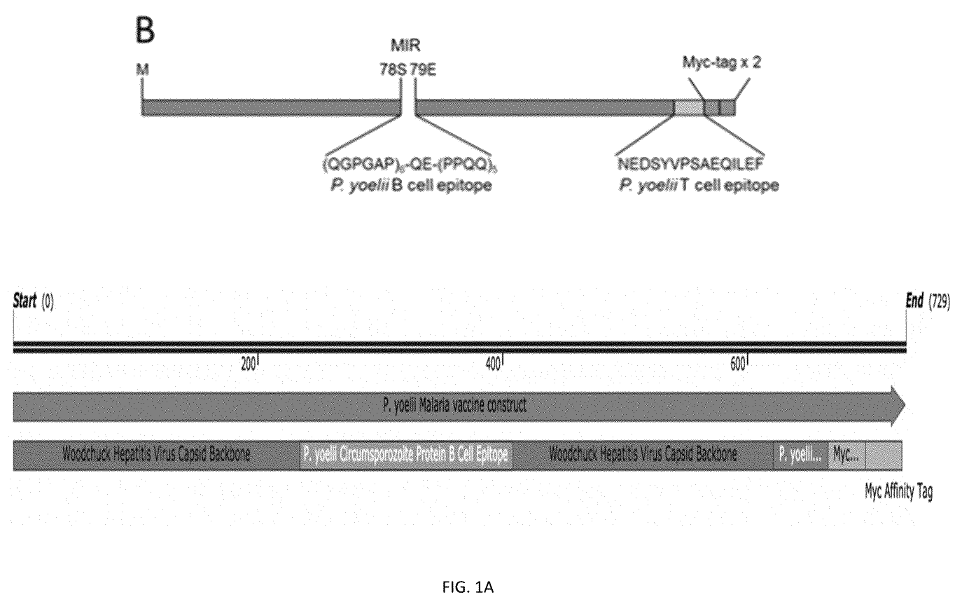

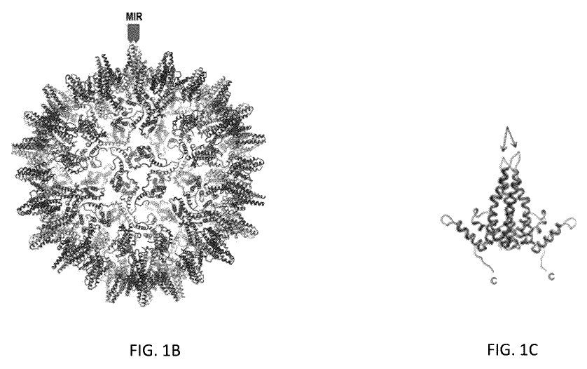

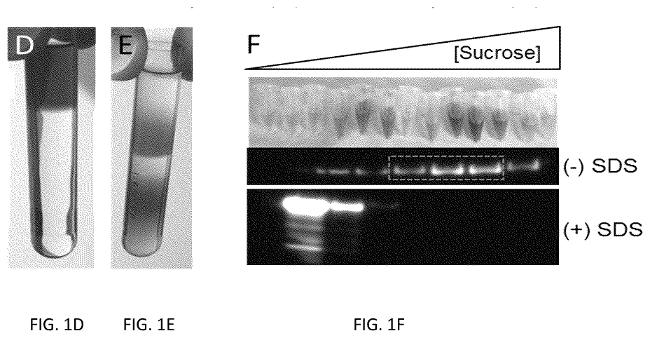

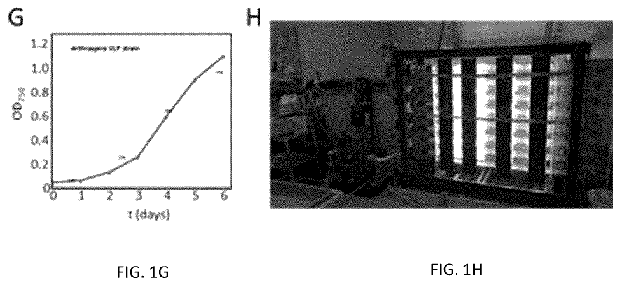

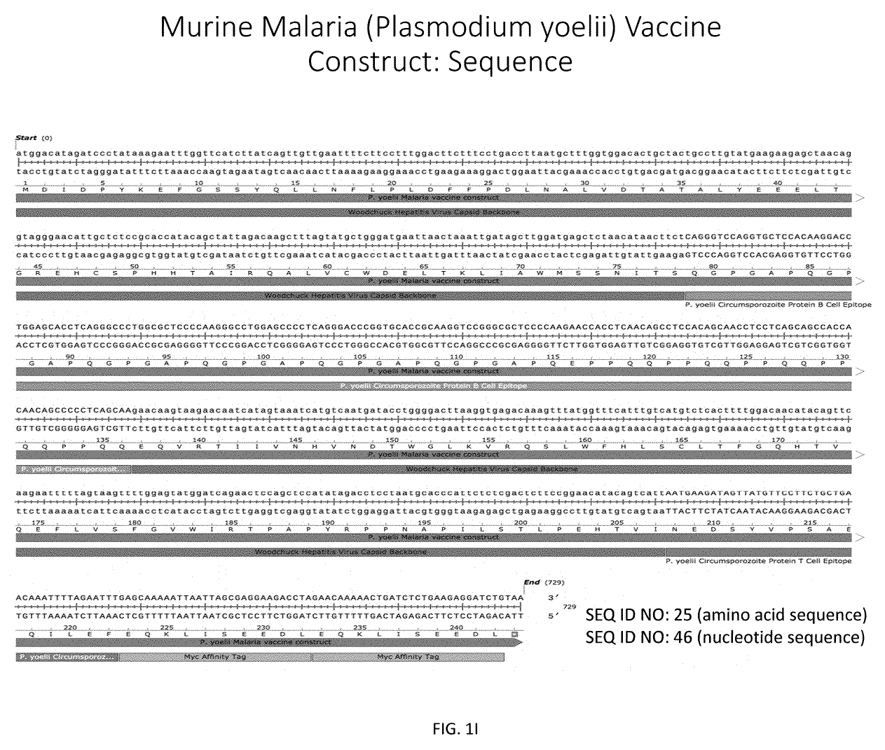

A DNA construct comprising a sequence encoding a homodimeric woodchuck hepadnavirus core antigen (WHcAg) fusion protein containing tandem repeats of P. yoelii circumsporozoite protein (CSP) B cell epitopes, and a CSP T cell epitope at the C-terminus followed by tandem myc tags was synthesized. FIG. 1A shows a schematic of the construct. As shown in the schematic, tandem repeats of P. yoelii CSP B cell epitopes were inserted at the Major Insertion Region (MIR), which is the region between the amino acid residues S78 and E79 of the WHcAg. The CSP T cell epitope was added at the C-terminus of WHcAg followed by tandem myc tags. FIG. 1I shows the sequence of the construct. The DNA construct encoding the above-described WHcAg fusion protein was introduced into Spirulina using a homologous recombination method. WHcAg homodimers assemble into VLPs of 90 or 120 units (FIG. 1B) and form a ‘spike’ containing the MIR (FIG. 1C) where the CSP B cell epitopes were inserted (arrows). The recombinant Spirulina expressing the construct was cultivated.

The Spirulina culture was sonicated and subjected to bioanalytical discontinuous sucrose density ultracentrifugation and fractionation (FIGS. 1D-F). Sonicated Spirulina culture before (FIG. 1D) and after (FIG. 1E) bioanalytical discontinuous sucrose density ultracentrifugation. After the centrifugation. the culture showed an orange carotenoid fraction (near the rim of the test tube), a blue phycocyanin fraction (in the middle) and a green chlorophyll pigments fraction (in the lower half of the test tube) with bottom drop fractions resolved by SDS-PAGE and Western blotted using anti-myc-HRP (FIG. IF). Detergent-free CSP-containing VLPs sediment at 60% sucrose (dashed box) and are abolished by SDS pre-treatment. These VLPs could be detected by spz hyperimmune sera (not shown). Recombinant Spirulina grew with wild-type growth kinetics (FIG. 1G). The process is scalable from the 1.5 L scale used here to 200 L scale using Lumen Bioscience's culture facilities (FIG. 1H).

### Example 2: Oral Spirulina Vaccination Induces Protective Anti-CSP IgG

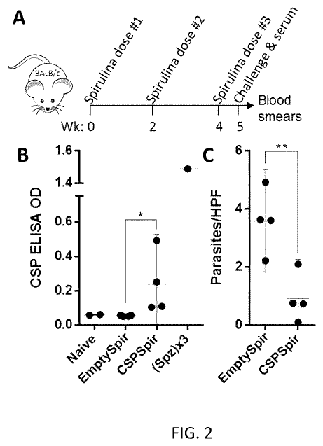

Naïve BALB/cj mice were vaccinated at 0, 2 and 4 weeks by oral gavage with 200 μL of a slurry containing 10 mg of lyophilized whole Spirulina biomass carrying 40 μg of the repeat domain of PyCSP in WHcAg VLPs described in Example 1 or empty WHcAg VLPs with Montanide IMS 1313 VG adjuvant in 0.2 M sodium bicarbonate. FIG. 2A shows the timeline for the experiment. Serum was collected at baseline and before each dose. One week after the third dose. serum was collected and mice intravenously (i.v.) challenged with 125 purified wild-type P. yoelii spz. Mice were followed post-challenge by thin blood smears for blood stage infection. A CSP ELISA assay was carried out on the serum. FIG. 2B shows the CSP ELISA data showing optical densities (OD) for serum from naïve mice or those immunized 3× with Spirulina carrying empty VLPs. Spirulina carrying CSP VLPs or attenuated spz. Half of Spirulina CSP-immunized mice seroconverted (FIG. 2B). FIG. 2C shows the Day 5 blood smear data showing mean parasites per high powered field; 50 HPF per mouse; *p<0.05; **p<0.01 (t-tests). CSP-immunized mice had lower onset parasite densities than control mice, indicating partial liver stage protection (FIG. 2C). Spirulina-primed antibodies were predominantly IgG (data not shown). These data is promising for two reasons. First, IgG was induced using an inert algae-based oral vaccine. Second, partial protection was observed despite using an i.v. challenge route that bypasses the opportunity for CSP-specific antibodies to block spz invasion of dermal blood vessels. Thus, this vaccination could be even more effective against intradermal or mosquito bite challenge.

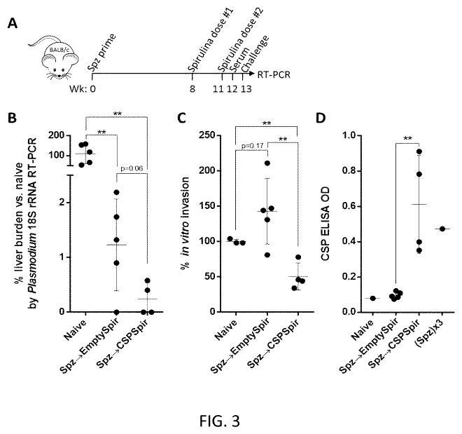

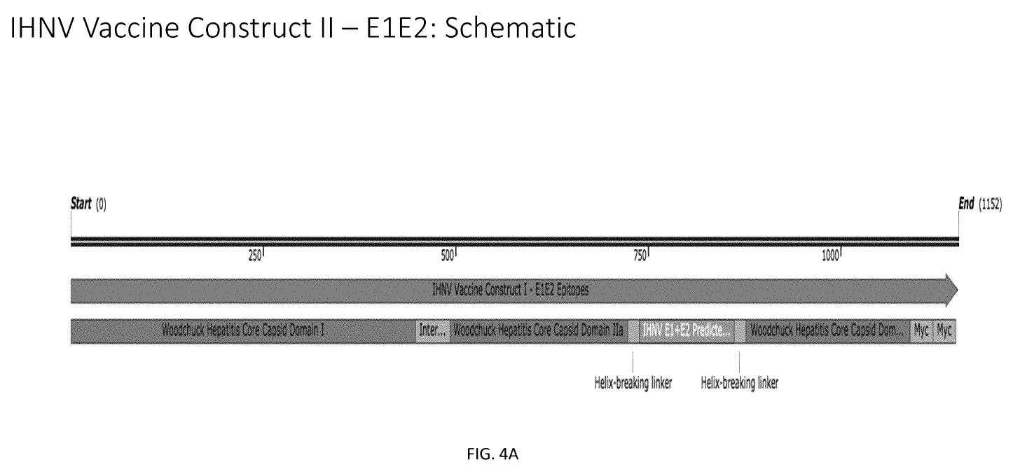

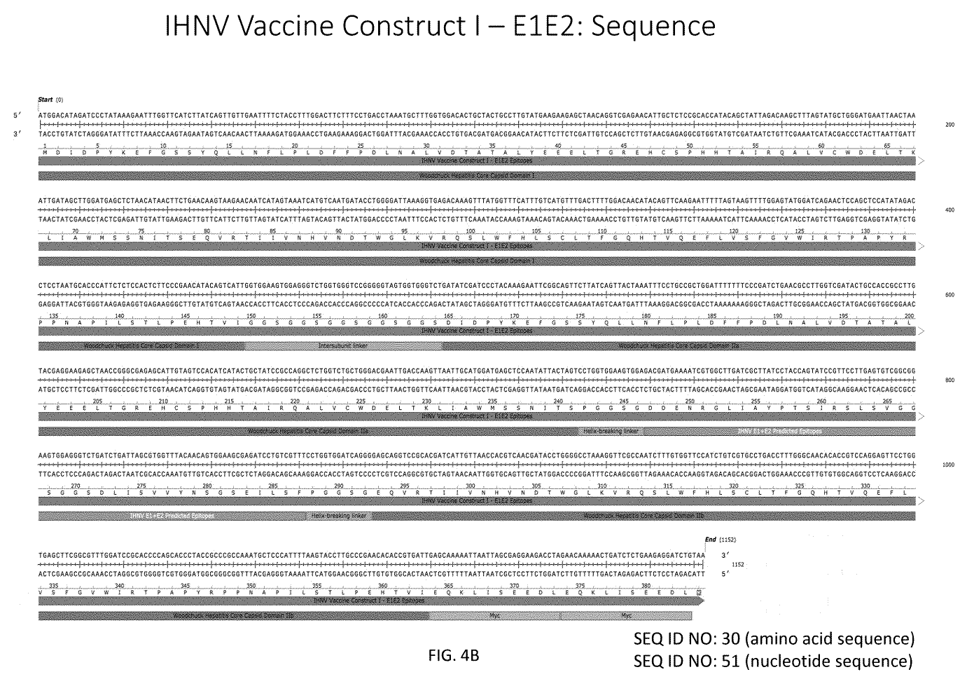

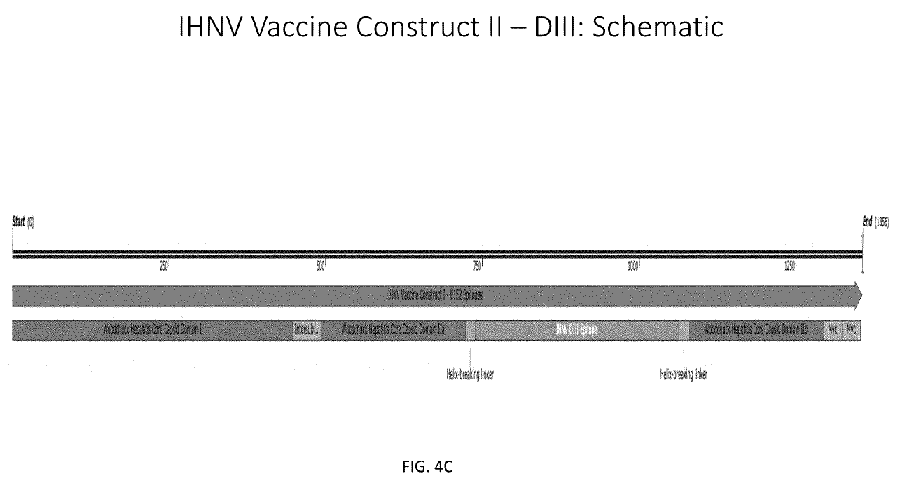

Since the seroconversion was not obtained in all Spirulina CSP VLP-immunized mice. a second experiment was performed to examine Spirulina-mediated boosting of spz-primed CSP-specific antibodies (FIG. 3A). BALB/cj mice were primed with 2×10⁴ purified irradiated P. yoelii spz and orally vaccinated with CSP—or empty Spirulina VLPs 8 and 11 wks later. Serum was collected throughout. Two weeks after the final Spirulina booster, mice (including a group of naïve infectivity control mice) were challenged i.v. with 2×10⁴ purified wild-type P. yoelii spz and protection was assessed two days later by liver RT-PCR for Plasmodium 18S rRNA. Compared to naïve mice, all spz-primed mice showed substantial reductions in liver burden as expected (FIG. 3B). Mice boosted with Spirulina CSP VLPs contained 6.3-fold less Plasmodium 18S rRNA than control VLP-boosted mice (p=0.06) and two mice showed undetectable Plasmodium 18S rRNA (the “RT-PCR equivalent” of sterile protection). In addition, serum from one week after the final booster dose showed potent activity in in vitro inhibition of spz invasion (ISI) assays (FIG. 3C) that was comparable to that seen in mice that can be protected against mosquito-bite challenge (data not shown) although such studies were not performed in our proof-of-concept work. CSP-specific titers were significantly increased in all spz-primed/Spirulina CSP VLP-boosted mice compared to spz-primed/Spirulina or control Spirulina-boosted mice and were comparable to CSP titers achieved in mice repeatedly exposed to attenuated spz. (FIG. 3D). As above, the boosted CSP-specific antibodies included IgG (not shown).

### Example 3: Spirulina Vaccine Comprising Canine Parvovirus Epitopes Induces Immune Response in Mice

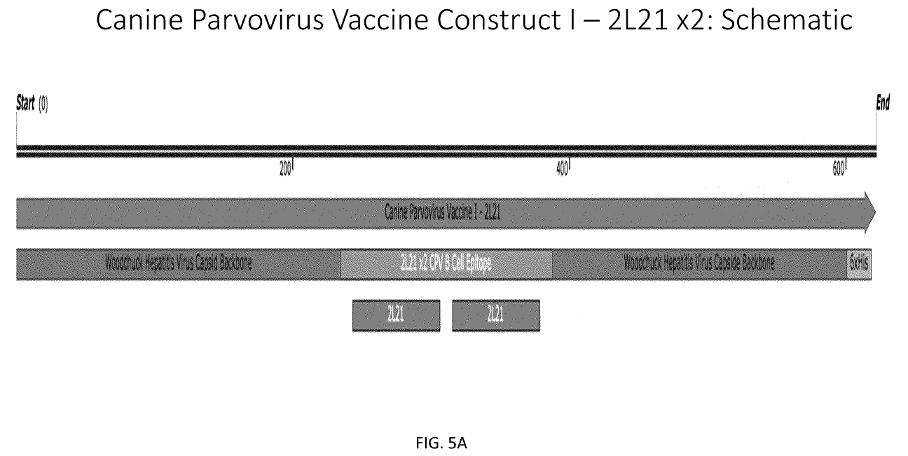

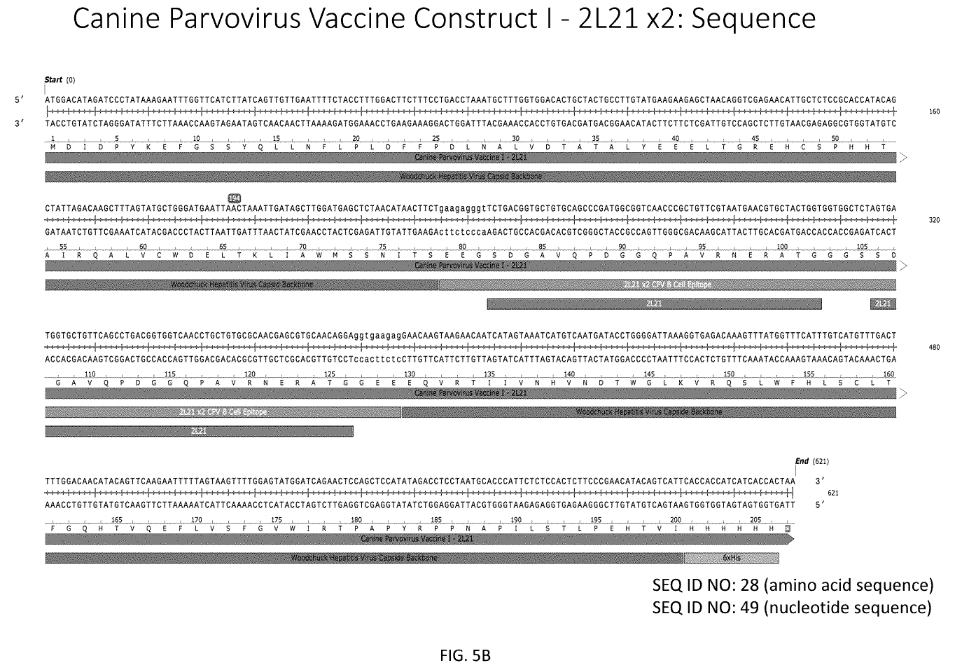

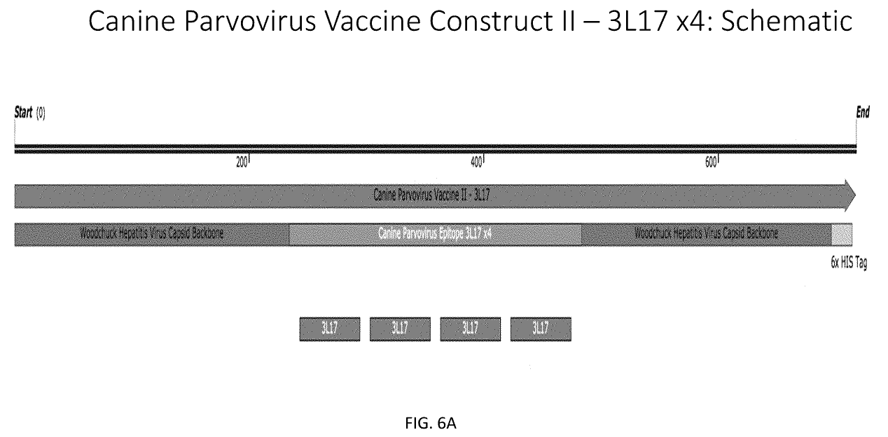

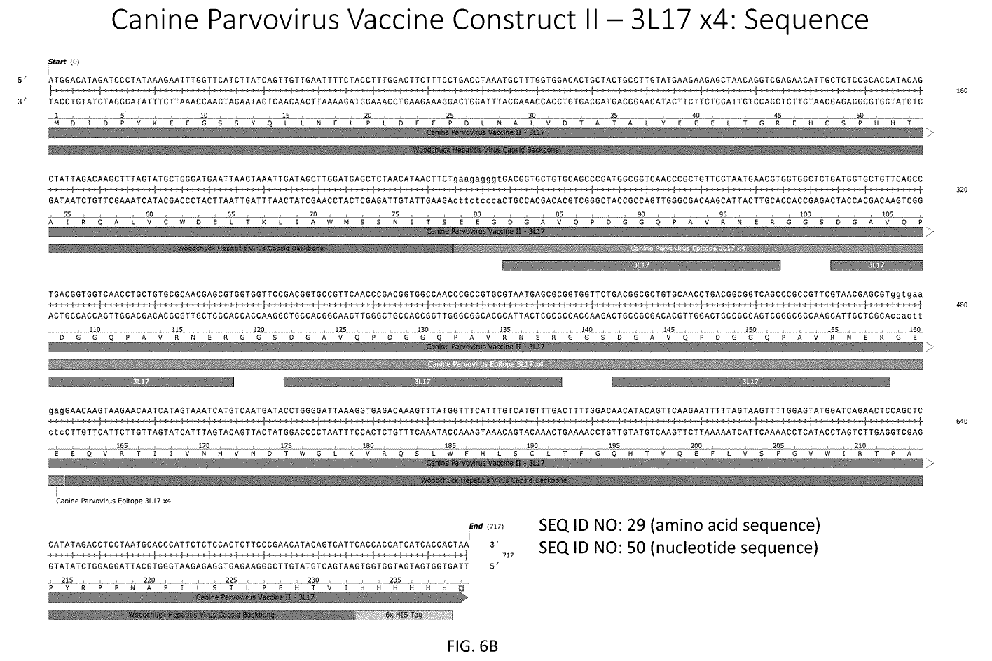

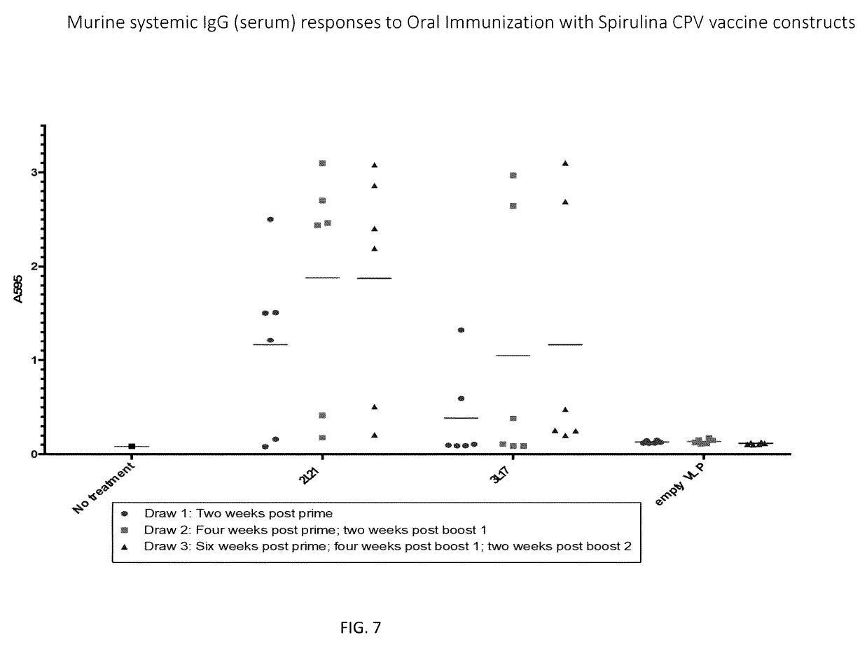

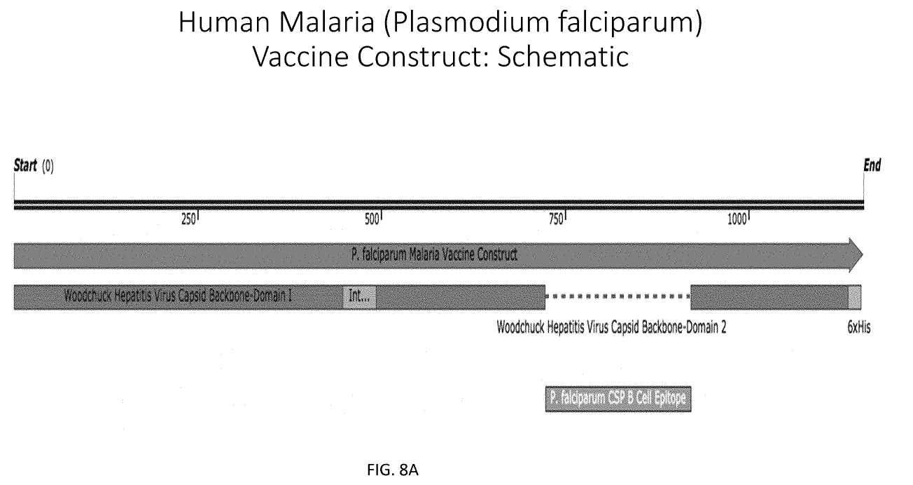

Spirulina were transformed with a vector comprising WHcAg and 2L21 B cell epitopes or WHcAg and 3L17 canine parovivirus epitopes. See FIGS. 5 and 6. Mice were orally vaccinated with a recombinant Spirulina slurry as taught in Example 2. Blood was drawn and tested for the presence of anti-canine parvovirus antibodies in the serum at two weeks post-priming (Draw 1); four weeks post-priming (Draw 2); and six weeks post-priming (Draw 3). FIG. 7 shows the murine systemic IgG responses to these Spriulina CPV vaccine constructs. Both constructs containing canine parvoviruses induced the production of serum IgG antibodies. No serum IgG antibodies were detected in either the “no treatment” or “empty VLP” groups.

### Example 4: Spirulina Vaccine Comprising Plasmodium Falciparum Epitopes Induces Immune Response in Mice

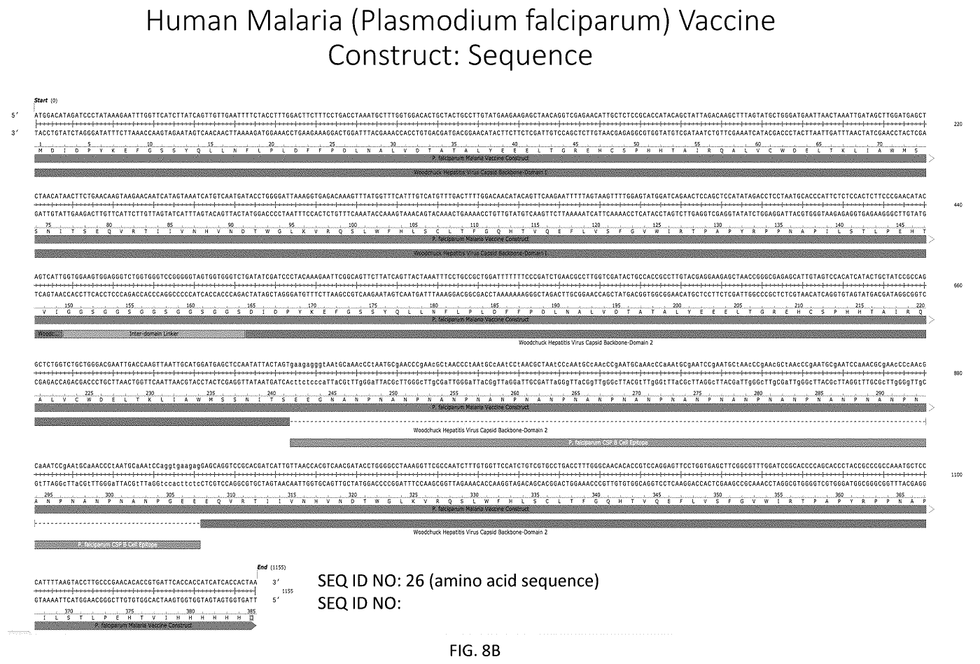

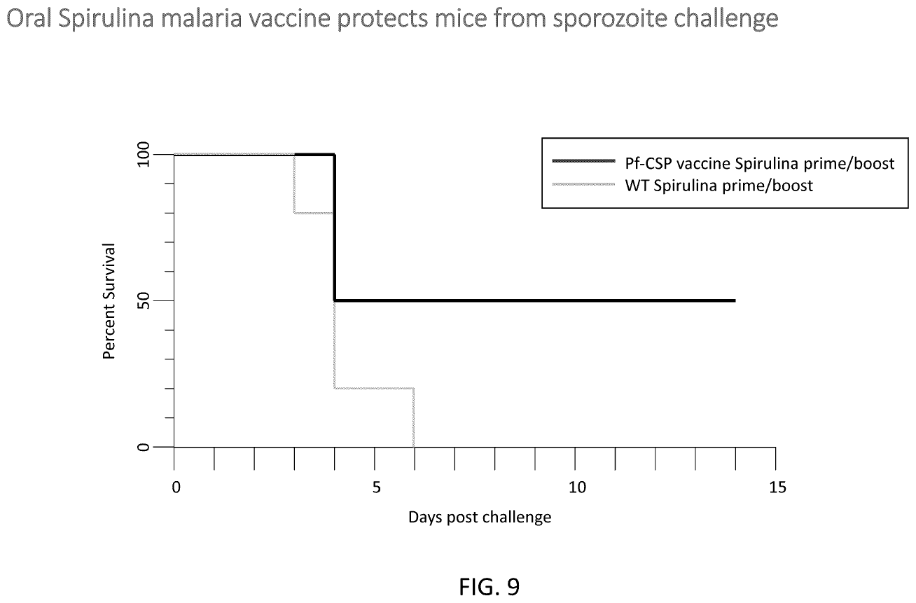

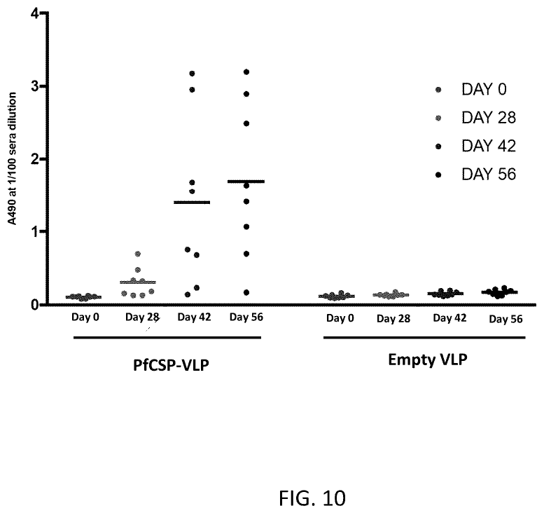

Spirulina were transformed with a vector comprising a nucleic acid sequence encoding a fusion protein comprising WHcAg domains and CSP B cell epitopes from Plasmodium falciparum. See FIG. 8. Mice were orally vaccinated with a wild type Spirulina or a recombinant Spirulina slurry as taught in Example 2. After the final boost, the mice were challenged (iv) with P. falciparum sporozoites, and the percent survival was measured up to 15 days post-challenge. The mice administered a wild type Spirulina were all dead by day 6. In contrast, those administered a Pf-CSP vaccine Spirulina showed greater survival, with 50% living for at least 15 days post-challenge. See FIG. 9. FIG. 10 shows that 7 out of 8 mice orally dosed with a Spirulina containing WHcAg particles with P. falciparum (NANPx) epitopes developed systemic IgG against P. falciparum, whereas mice orally dosed with a Spirulina containing empty WHcAg particles (without P. falciparum epitopes) did not develop any IgG response.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A method of reducing severity of an infection in a subject in need thereof, comprising administering to the subject an oral antigenic composition comprising:
a recombinant Spirulina, wherein the recombinant Spirulina comprises an exogenous nucleic acid sequence encoding a chimeric protein comprising an antigenic epitope translationally fused to a viral protein or a virus-like particle (VLP)-forming protein, wherein the exogenous nucleic acid sequence is stably integrated into an endogenous locus of the Spirulina;
wherein the antigenic epitope in the chimeric protein is from an infectious microorganism causing the infection.

2. The method of claim 1, wherein the viral protein or VLP-forming protein comprises a capsid protein of a virus.

3. The method of claim 2, wherein the capsid protein is hepatitis B core antigen (HbcAg) or woodchuck hepadnaviridae core antigen (WhcAg).

4. The method of claim 1, wherein the chimeric protein comprises a scaffold protein, and wherein the antigenic epitope is linked to the scaffold protein at the N-terminus or the C-terminus, or in the body of the scaffold protein.

5. The method of claim 1, wherein the recombinant Spirulina is non-living, dried, spray dried, freeze-dried, or lyophilized.

6. The method of claim 1, wherein the infection is selected from the group consisting of malaria, tetanus, diphtheria, pertussis, pneumonia, meningitis, campylobacteriosis, mumps, measles, rubella, polio, flu, hepatitis, chickenpox, malaria, toxoplasmosis, giardiasis, and leishmaniasis.

7. The method of claim 1, wherein the infectious microorganism is a virus selected from the group consisting of: bacteriophage, RNA bacteriophage, Infectious Haematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, and Poliovirus, Norovirus, Zika virus, Dengue Virus, Rabies Virus, Newcastle Disease Virus, and White Spot Syndrome Virus.

8. The method of claim 1, wherein the infectious microorganism is a parasite selected from the group consisting of: Plasmodium, Trypanosoma, Toxoplasma, Giardia, Leishmania, Cryptosporidium, helminthic parasites: Trichuris spp., Enterobius spp., Ascaris spp., Ancylostoma spp., Necator spp., Strongyloides spp., Dracunculus spp., Onchocerca spp. And Wuchereria spp., Taenia spp., Echinococcus spp., and Diphyllobothrium spp., Fasciola spp., and Schistosoma spp.

9. The method of claim 8, wherein the infectious microorganism is a Plasmodium and the antigenic epitope is selected from the group consisting of: circumsporozoite protein, thrombospondin-related anonymous protein (TRAP), Apical Membrane Antigen 1 (AMA1), major merozoite surface proteins 1-3 (MSP1-3), sexual stage antigen 25 (s25), sexual stage antigen s230, and a sequence of NANP (SEQ ID NO: 6), NVDP (SEQ ID NO:7), or NPDP (SEQ ID NO: 8).

10. The method of claim 1, wherein the antigenic epitope is from a circumsporozoite protein of a Plasmodium.

11. The method of claim 1, wherein the antigenic epitope comprises the sequence of NANP (SEQ ID NO: 6).

12. The method of claim 1, comprising administering a priming dose of the oral antigenic composition and subsequently administering one or more booster doses of the oral antigenic composition.


SEQUENCE LISTING (70 sequences)

SEQ ID NO: 1 - protein, 65 aa, synthetic construct
  REGION 1..65: note = oligomerization domain
SAGAHAGWETPEGCEQVLTGKRLMQCLPNPEDVKMALEVYKLSLEIEQLELQRDSARQSTLDKEL

SEQ ID NO: 2 - protein, 55 aa, synthetic construct
  REGION 1..55: note = oligomerization domain
WVIPEGCGHVLAGRKVMQCLPNPEDVKMALEVYKLSLEIELLEIQRDKARDPAMD

SEQ ID NO: 3 - protein, 52 aa, synthetic construct
  REGION 1..52: note = oligomerization domain
WEYAEGCEQVVKGKKLMQCLPTPEEVRLALEVYKLYLEIQKLELQKDEAKQA

SEQ ID NO: 4 - protein, 58 aa, synthetic construct
  REGION 1..58: note = oligomerization domain
WVVPAGCEQVIAGRELTQCLPSVEDVKMALELYKLSLEIELLELQKDKAKKSTLESPL

SEQ ID NO: 5 - protein, 16 aa, synthetic construct
  REGION 1..16: note = tandem linked antigenic epitope
NANPNANPNANPNANP

SEQ ID NO: 6 - protein, 4 aa, Plasmodium falciparum
NANP

SEQ ID NO: 7 - protein, 4 aa, Plasmodium falciparum
NVDP

SEQ ID NO: 8 - protein, 4 aa, Plasmodium falciparum
NPDP

SEQ ID NO: 9 - protein, 40 aa, Plasmodium falciparum
KLKQPGDGNPDPNANPNVDPNANPNVDPNANPNVDPNANP

SEQ ID NO: 10 - protein, 20 aa, Plasmodium falciparum
EYLKKIQNSLSTEWSPCSVT

SEQ ID NO: 11 - protein, 60 aa, Plasmodium falciparum
NANPNANPNANPNANPNANPNANPNANPNANPNANPNANPNANPNANPNANPNANPNANP

SEQ ID NO: 12 - protein, 35 aa, Plasmodium falciparum
GDGNPDPNANPNVDPNANPNVDPNANPNVDPNANP

SEQ ID NO: 13 - protein, 91 aa, Plasmodium falciparum
GDGNPDPNANPNVDPNANPNVDPNANPNVDPNANPNANPNANPNANPNANPNANPNANPNANPNANPNANPNANPNANPNANPNANPNANP

SEQ ID NO: 14 - protein, 112 aa, Plasmodium falciparum
DGNNEDNEKLRKPKHKKLKQPGDGNPDPNANPNVDPNANPNVDPNANPNVDPNANPNANPNANPNANPNANPNANPNANPNANPNANPNANPNANPNANPNANPNANPNANP

SEQ ID NO: 15 - protein, 7 aa, Plasmodium falciparum
HPSDGKC

SEQ ID NO: 16 - protein, 4 aa, Plasmodium falciparum
DRYI

SEQ ID NO: 17 - protein, 6 aa, Plasmodium falciparum
TRPHGR

SEQ ID NO: 18 - protein, 58 aa, Plasmodium yoelii
QGPGAPQGPGAPQGPGAPQGPGAPQGPGAPQGPGAPQEPPQQPPQQPPQQPPQQPPQQ

SEQ ID NO: 19 - protein, 15 aa, Plasmodium yoelii
NEDSYVPSAEQILEF

SEQ ID NO: 20 - protein, 66 aa, Plasmodium falciparum
EEGNANPNANPNANPNANPNANPNANPNANPNANPNANPNANPNANPNANPNANPNANPNANPGEE

SEQ ID NO: 21 - protein, 21 aa, unidentified
  source 1..21: note = Canine parvovirus
SDGAVQPDGGQPAVRNERATG

SEQ ID NO: 22 - protein, 17 aa, unidentified
  source 1..17: note = Canine parvovirus
DGAVQPDGGQPAVRNER

SEQ ID NO: 23 - protein, 41 aa, synthetic construct
  REGION 1..41: note = IHNV E1+E2 epitopes
DDENRGLIAYPTSIRSLSVGGSGGSDLISVVYNSGSEILSF

SEQ ID NO: 24 - protein, 108 aa, synthetic construct
  REGION 1..108: note = IHNV DIII epitope
DDENRGLIAYPTSIRSLSVGNDGGSGGSSQEIKAHLFVDKISNRVVKATSYGHHPWGLHQACMIEFCGQQWIRTDLGDLISVVYNSGSEILSFPKCEDKTVGPAEGGG

SEQ ID NO: 25 - protein, 242 aa, synthetic construct
  REGION 1..242: note = P. yoelii CSP B cell epitope in monomer WHcAg
MDIDPYKEFGSSYQLLNFLPLDFFPDLNALVDTATALYEEELTGREHCSPHHTAIRQALVCWDELTKLIAWMSSNITSQGPGAPQGPGAPQGPGAPQGPGAPQGPGAPQGPGAPQEPPQQPPQQPPQQPPQQPPQQEQVRTIIVNHVNDTWGLKVRQSLWFHLSCLTFGQHTVQEFLVSFGVWIRTPAPYRPPNAPILSTLPEHTVINEDSYVPSAEQILEFEQKLISEEDLEQKLISEEDL

SEQ ID NO: 26 - protein, 384 aa, synthetic construct
  REGION 1..384: note = P. falciparum CSP B cell epitope in tandem WHcAg dimer
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIGGSGGSGGSGGSGGSDIDPYKEFGSSYQLLNFLPLDFFPDLNALVDTATALYEEELTGREHCSPHHTAIRQALVCWDELTKLIAWMSSNITSEEGNANPNANPNANPNANPNANPNANPNANPNANPNANPNANPNANPNANPNANPNANPNANPGEEEQVRTIIVNHVNDTWGLKVRQSLWFHLSCLTFGQHTVQEFLVSFGVWIRTPAPYRPPNAPILSTLPEHTVIHHHHHH

SEQ ID NO: 27 - protein, 656 aa, synthetic construct
  REGION 1..656: note = P. falciparum CSP B cell epitope + Salmonella fliC sequences in tandem WHcAg dimer
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIGGSGGSGGSGGSGGSDIDPYKEFGSSYQLLNFLPLDFFPDLNALVDTATALYEEELTGREHCSPHHTAIRQALVCWDELTKLIAWMSSNITSEEGAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQGSNANPNANPNANPNANPNANPNANPNANPNANPNANPNANPNANPNANPNANPNANPNANPGSTTTENPLQKIDAALAQVDTLRSDLGAVQNRFNSAITNLGNTVNNLTSARSRIEDSDYATEVSNMSRAQILQQAGTSVLAQANQVPQNVLSLLRGEEEQVRTIIVNHVNDTWGLKVRQSLWFHLSCLTFGQHTVQEFLVSFGVWIRTPAPYRPPNAPILSTLPEHTVIHHHHHH

SEQ ID NO: 28 - protein, 206 aa, synthetic construct
  REGION 1..206: note = Canine parvovirus 2L21 peptide epitope in 2x configuration; monomer WhcAg
MDIDPYKEFGSSYQLLNFLPLDFFPDLNALVDTATALYEEELTGREHCSPHHTAIRQALVCWDELTKLIAWMSSNITSEEGSDGAVQPDGGQPAVRNERATGGGSSDGAVQPDGGQPAVRNERATGGEEEQVRTIIVNHVNDTWGLKVRQSLWFHLSCLTFGQHTVQEFLVSFGVWIRTPAPYRPPNAPILSTLPEHTVIHHHHHH

SEQ ID NO: 29 - protein, 238 aa, synthetic construct
  REGION 1..238: note = Canine parvovirus 3L17 peptide epitope in 4x configuration; monomer WhcAg
MDIDPYKEFGSSYQLLNFLPLDFFPDLNALVDTATALYEEELTGREHCSPHHTAIRQALVCWDELTKLIAWMSSNITSEEGDGAVQPDGGQPAVRNERGGSDGAVQPDGGQPAVRNERGGSDGAVQPDGGQPAVRNERGGSDGAVQPDGGQPAVRNERGEEEQVRTIIVNHVNDTWGLKVRQSLWFHLSCLTFGQHTVQEFLVSFGVWIRTPAPYRPPNAPILSTLPEHTVIHHHHHH

SEQ ID NO: 30 - protein, 383 aa, synthetic construct
  REGION 1..383: note = IHNV E1+E2 epitopes in tandem WHcAg dimer
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIGGSGGSGGSGGSGGSDIDPYKEFGSSYQLLNFLPLDFFPDLNALVDTATALYEEELTGREHCSPHHTAIRQALVCWDELTKLIAWMSSNITSPGGSGDDENRGLIAYPTSIRSLSVGGSGGSDLISVVYNSGSEILSFPGGSGEQVRTIIVNHVNDTWGLKVRQSLWFHLSCLTFGQHTVQEFLVSFGVWIRTPAPYRPPNAPILSTLPEHTVIEQKLISEEDLEQKLISEEDL

SEQ ID NO: 31 - protein, 451 aa, synthetic construct
  REGION 1..451: note = IHNV DIII epitope in tandem WHcAg dimer
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIGGSGGSGGSGGSGGSDIDPYKEFGSSYQLLNFLPLDFFPDLNALVDTATALYEEELTGREHCSPHHTAIRQALVCWDELTKLIAWMSSNITSPGGSGDDENRGLIAYPTSIRSLSVGNDGGSGGSSQEIKAHLFVDKISNRVVKATSYGHHPWGLHQACMIEFCGQQWIRTDLGDLISVVYNSGSEILSFPKCEDKTVGPAEGGGPAGGSGEQVRTIIVNHVNDTWGLKVRQSLWFHLSCLTFGQHTVQEFLVSFGVWIRTPAPYRPPNAPILSTLPEHTVIEQKLISEEDLEQKLISEEDL

SEQ ID NO: 32 - protein, 431 aa, Plasmodium falciparum
MRKLAILSVSSFLFVEALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNDVKNNNNEEPSDKHIEEYLKKIQNSLSTEWSPCSVTCGNGIQVRIKPGSANKPKDELNYENDIEKKICKMEKCSSVFNVVNSSIGLIMVLSFLFLN

SEQ ID NO: 33 - protein, 434 aa, Plasmodium falciparum
MRKLAILSVSSFLFVEALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNDENNNNEEPSDKHIEQYLKKIQYSLSTEWSPCSVTCGNGIQVRIKPGSADKPKDQLDYENDIEKKICKMEKCSSVFNVVNSSIGLIMVLSFLFLN

SEQ ID NO: 34 - protein, 423 aa, Plasmodium falciparum
MRKLAILSVSSFLFVEALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIQNSLSTEWSPCSVTCGNGIQVRIKPGSANKPKDELDYENDIEKKICKMEKCSSVFNVVNSSIGLIMVLSFLFLN

SEQ ID NO: 35 - protein, 405 aa, Plasmodium falciparum
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 36 - protein, 427 aa, Plasmodium falciparum
MRKLAILSVSSFLFVEALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIQNSLSTEWSPCSVTCGNGIQVRIKPGSANKPKDELDYENDIEKKICKMEKCSSVFNVVNSSIGLIMVLSFLFLN

SEQ ID NO: 37 - protein, 428 aa, Plasmodium falciparum
MMRKLAILSVSSFLFVEALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNVDPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIQNSLSTEWSPCSVTCGNGIQVRIKPGSANKPKDELDYENDIEKKICKMEKCSSVFNVVNSSIGLIMVLSFLFLN

SEQ ID NO: 38 - protein, 416 aa, Plasmodium falciparum
MMRKLAILSVSSFLFVEALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIQNSLSTEWSPCSVTCGNGIQVRIKPGSANKPKDELDYENDIEKKICKMEKCSSVFNVVNSSIGLIMVLSFLFLN

SEQ ID NO: 39 - protein, 416 aa, Plasmodium falciparum; identical to SEQ ID NO: 38

SEQ ID NO: 40 - protein, 412 aa, Plasmodium falciparum
MMRKLAILSVSSFLFVEALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIQNSLSTEWSPCSVTCGNGIQVRIKPGSADKPKDQLDYENDIEKKICKMEKCSSVFNVVNSSIGLIMVLSFLFLN

SEQ ID NO: 41 - protein, 564 aa, Plasmodium falciparum
MNHLGNVKYLVIVFLIFFDLFLVNGRDVQNNIVDEIKYREEVCNDEVDLYLLMDCSGSIRRHNWVKHAVPLAMKLIQQLNLNENAIHLYLNIFSNAREIIRLHSDASKNKEKALIIIKSLLNTNLPYGRTNLTDALLQVRKHLNDRINRENANQLVVILTDGIPDSIQDSLKESRKLNDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCPPKREPLDVPDEPEDDQPRPRGDNFAVEKPEENIIDNNPQEPSPNPEEGKGENPNGFDLDENPENPPNPPNPPNPDIPEQEPNIPEDSEKEVPSDVPKNPEDDREENFDIPKKPENKHDNQNNLPNDKSDRYIPYSPLPPKVLDNERKQSDPQSQDNNGNRHVPNSEDRETRPHGRNNENRSYNRKHNNTPKHPEREEHEKPDNNKKKGGSDNKYKIAGGIAGGLALLACAGLAYKFVVPGAATPYAGEPAPFDETLGEEDKDLDEPEQFRLPEENEWN

SEQ ID NO: 42 - protein, 559 aa, Plasmodium falciparum
MNHLGNVKYLVIVFLIFFDLFLVNGRDVQNNIVDEIKYREEVCNDEVDLYLLMDCSGSIRRHNWVKHAVPLAMKLIQQLNLNENAIHLYVNIFSNNAKEIIRLHSDASKNKEKALIIIKSLLSTNLPYGRTNLSDALLQVRKHLNDRINRENANQLVVILTDGIPDSIQDSLKESRKLNDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCPPKREPLDVPDEPEDDQPRPRGDNFAVEKPEENIIDNNPQEPSPNPEEGKGENPNGFDLDENPENPPNPDIPEQEPNIPEDSEKEVPSDVPKNPEDDREENFDIPKKPENKHDNQNNLPNDKSDRSIPYSPLPPKVLDNERKQSDPQSQDNNGNRHVPNSEDRETRPHGRNNENRSYNRKYNDTPKHPEREEHEKPDNNKKKGGSDNKYKIAGGIAGGLALLACAGLAYKFVVPGAATPYAGEPAPFDETLGEEDKDLDEPEQFRLPEENEWN

SEQ ID NO: 43 - protein, 559 aa, Plasmodium falciparum
MNHLGNVKYLVIVFLIFFDLFLVNGRDVQNNIVDEIKYSEEVCNDQVDLYLLMDCSGSIRRHNWVNHAVPLAMKLIQQLNLNDNAIHLYVNVFSNNAKEIIRLHSDASKNKEKALIIIRSLLSTNLPYGRTNLTDALLQVRKHLNDRINRENANQLVVILTDGIPDSIQDSLKESRKLSDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSEIQEQCEEERCPPKWEPLDVPDEPEDDQPRPRGDNSSVQKPEENIIDNNPQEPSPNPEEGKDENPNGFDLDENPENPPNPDIPEQKPNIPEDSEKEVPSDVPKNPEDDREENFDIPKKPENKHDNQNNLPNDKSDRNIPYSPLPPKVLDNERKQSDPQSQDNNGNRHVPNSEDRETRPHGRNNENRSYNRKYNDTPKHPEREEHEKPDNNKKKGESDNKYKIAGGIAGGLALLACAGLAYKFVVPGAATPYAGEPAPFDETLGEEDKDLDEPEQFRLPEENEWN

SEQ ID NO: 44 - protein, 559 aa, Plasmodium falciparum
MNHLGNVKYLVIVFLIFFDLFLVNGRDVQNNIVDEIKYREEVCNDEVDLYLLMDCSGSIRRHNWVKHAVPLAMKLIQQLNLNESAIHLYVNIFSNNAREIIRLHSDASKNKEKALIIIKSLLSTNLPYGRTNLTDALLQVRKHLNDRINRENASQLVVILTDGIPDSIQDSLKESRKLNDLGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCPPKREPLDVPHEPEDDQPRPRGDNFAVEKPKENIIDNNPQEPSPNPEEGKGENPNGFDLDENPENPPNPDIPEQEPNIPEDSEKEVPSDVPKNPEDDREENFDIPKKPENKHDNQNNLPNDKSDRSIPYSPLPPKVLDNERKQSDPQSQDNNGNRHVPNSEDRETRPHGRNNENRSYNRKYNDTPKHPEREEHEKPDNNKKKGGSDNKYKIAGGIAGGLALLACAGLAYKFVVPGAATPYAGEPAPFDETLGEEDKDLDEPEQFRLPEENEWN

SEQ ID NO: 45 - protein, 568 aa, Plasmodium falciparum
MNHLGNVKYLVIVFLIFFDLFLVNGRDVQNNIVDEIKYREEVCNDEVDLYLLMDCSGSIRRHNWVNHAVPLAMKLIQQLNLNESAIHLYLNIFSNNAREIIRLHSDASKNKEKALIIIKSLLNTNLPYGRTNLTDALLQVRKHLNDRINRENANQLVVILTDGIPDSIQDSLKESRKLNDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCPPKREPLDVPHEPEDDQPRPRGDNFVVEKPEENIIDNNPQEPSPNPEEGKGENPNGFDLDENPENPPNPPNPPNPPNPDIPEQEPNIPEDSEKEVPSDVPKNPEDDREENFDIPKKPENKHDNQNNLPNDKSDRYIPYSPLPPKVLDNERKQSDPQSQDNNGNRHVPNSEDRETRPHGRNNENRSYNRKHNNTPKHPEREEHEKPDNNKKKGGSDNKYKIAGGIAGGLALLACAGLAYKFVVPGAATPYAGEPAPFDETLGEEDKDLDEPEQFRLPEENEWN

SEQ ID NO: 46 - DNA, 729 bp, synthetic construct
  misc_feature 1..729: note = P. yoelii CSP B cell epitope in monomer WHcAg
  source 1..729: mol_type = other DNA
atggacatagatccctataaagaatttggttcatcttatcagttgttgaattttcttcctttggacttctttcctgaccttaatgctttggtggacactgctactgccttgtatgaagaagagctaacaggtagggaacattgctctccgcaccatacagctattagacaagctttagtatgctgggatgaattaactaaattgatagcttggatgagctctaacataacttctcagggtccaggtgctccacaaggacctggagcacctcagggccctggcgctccccaagggcctggagcccctcagggacccggtgcaccgcaaggtccgggcgctccccaagaaccacctcaacagcctccacagcaacctcctcagcagccaccacaacagccccctcagcaagaacaagtaagaacaatcatagtaaatcatgtcaatgatacctggggacttaaggtgagacaaagtttatggtttcatttgtcatgtctcacttttggacaacatacagttcaagaatttttagtaagttttggagtatggatcagaactccagctccatatagacctcctaatgcacccattctctcgactcttccggaacatacagtcattaatgaagatagttatgttccttctgctgaacaaattttagaatttgagcaaaaattaattagcgaggaagacctagaacaaaaactgatctctgaagaggatctgtaa

SEQ ID NO: 47 - DNA, 1155 bp, synthetic construct
  misc_feature 1..1155: note = P. falciparum CSP B cell epitope in tandem WHcAg dimer
  source 1..1155: mol_type = other DNA
atggacatagatccctataaagaatttggttcatcttatcagttgttgaattttctacctttggacttctttcctgacctaaatgctttggtggacactgctactgccttgtatgaagaagagctaacaggtcgagaacattgctctccgcaccatacagctattagacaagctttagtatgctgggatgaattaactaaattgatagcttggatgagctctaacataacttctgaacaagtaagaacaatcatagtaaatcatgtcaatgatacctggggattaaaggtgagacaaagtttatggtttcatttgtcatgtttgacttttggacaacatacagttcaagaatttttagtaagttttggagtatggatcagaactccagctccatatagacctcctaatgcacccattctctccactcttcccgaacatacagtcattggtggaagtggagggtctggtgggtccgggggtagtggtgggtctgatatcgatccctacaaagaattcggcagttcttatcagttactaaatttcctgccgctggatttttttcccgatctgaacgccttggtcgatactgccaccgccttgtacgaggaagagctaaccgggcgagagcattgtagtccacatcatactgctatccgccaggctctggtctgctgggacgaattgaccaagttaattgcatggatgagctccaatattactagtgaagagggtaatgcaaaccctaatgcgaacccgaacgctaaccctaatgccaatcctaacgctaatcccaatgccaacccgaatgcaaacccaaatgcgaatccgaatgctaacccgaacgctaacccgaatgcgaatccaaacgcgaaccccaacgcaaatccgaatgcaaaccctaatgcaaatccaggtgaagaggagcaggtccgcacgatcattgttaaccacgtcaacgatacctggggcctaaaggttcgccaatctttgtggttccatctgtcgtgcctgacctttgggcaacacaccgtccaggagttcctggtgagcttcggcgtttggatccgcaccccagcaccctaccgcccgccaaatgctcccattttaagtaccttgcccgaacacaccgtgattcaccaccatcatcaccactaa

SEQ ID NO: 48 - DNA, 1971 bp, synthetic construct
  misc_feature 1..1971: note = P. falciparum CSP B cell epitope + Salmonella fliC sequences in tandem WHcAg dimer
  source 1..1971: mol_type = other DNA
atggacatagatccctataaagaatttggttcatcttatcagttgttgaattttctacctttggacttctttcctgacctaaatgctttggtggacactgctactgccttgtatgaagaagagctaacaggtcgagaacattgctctccgcaccatacagctattagacaagctttagtatgctgggatgaattaactaaattgatagcttggatgagctctaacataacttctgaacaagtaagaacaatcatagtaaatcatgtcaatgatacctggggattaaaggtgagacaaagtttatggtttcatttgtcatgtttgacttttggacaacatacagttcaagaatttttagtaagttttggagtatggatcagaactccagctccatatagacctcctaatgcacccattctctccactcttcccgaacatacagtcattggtggaagtggagggtctggtgggtccgggggtagtggtgggtctgatatcgatccctacaaagaattcggcagttcttatcagttactaaatttcctgccgctggatttttttcccgatctgaacgccttggtcgatactgccaccgccttgtacgaggaagagctaaccgggcgagagcattgtagtccacatcatactgctatccgccaggctctggtctgctgggacgaattgaccaagttaattgcatggatgagctccaatattactagtgaagagggtgcgcaggtcattaacactaattcgctgagtttactaacacagaataatctaaacaagagccaatccgcccttggcacggcgatcgagcggctgagttcggggctgcgtatcaatagtgccaaagatgacgcggccggccaggcgattgcaaatcgttttacggccaatataaaggggcttacacaggcttctcgtaatgccaacgacggtatttccattgcccaaacaacggaaggcgcgctgaatgaaatcaataataatctgcagcgggtccgggagttagcggtgcagtctgccaactcaacaaattctcaatcagatctggattctatccaggcagaaataactcagaggcttaatgaaatcgatcgtgtttctggacaaacccagtttaatggtgtcaaggtccttgctcaggacaacaccctgaccatccaggtaggcgcgaacgatggagaaaccattgatattgatctgaaacagattaattctcagactctaggtcttgacaccttgaatgtgcagggttctaatgcaaaccctaatgcgaacccgaacgctaaccctaatgccaatcctaacgctaatcccaatgccaacccgaatgcaaacccaaatgcgaatccgaatgctaacccgaacgctaacccgaatgcgaatccaaacgcgaaccccaacgcaaatccgaatgcaaaccctaatgcaaatccaggttctaccacaaccgagaatcctctgcagaaaatcgatgctgctctcgcgcaagtggacactttgcgttcagatttgggagctgtgcaaaatcgtttcaacagcgcgattacaaacctgggtaacaccgtaaacaatctgactagtgcccggagtcggattgaagatagcgattatgcgaccgaagtgtctaacatgagccgggcccaaatcttgcagcaagccggcactagtgttctggcgcaagcaaatcaggtcccccaaaacgttctcagccttctgcggggtgaagaggagcaggtccgcacgatcattgttaaccacgtcaacgatacctggggcctaaaggttcgccaatctttgtggttccatctgtcgtgcctgacctttgggcaacacaccgtccaggagttcctggtgagcttcggcgtttggatccgcaccccagcaccctaccgcccgccaaatgctcccattttaagtaccttgcccgaacacaccgtgattcaccaccatcatcaccactaa

SEQ ID NO: 49 - DNA, 621 bp, synthetic construct
  misc_feature 1..621: note = Canine parvovirus 2L21 peptide epitope in 2x configuration; monomer WhcAg
  source 1..621: mol_type = other DNA
atggacatagatccctataaagaatttggttcatcttatcagttgttgaattttctacctttggacttctttcctgacctaaatgctttggtggacactgctactgccttgtatgaagaagagctaacaggtcgagaacattgctctccgcaccatacagctattagacaagctttagtatgctgggatgaattaactaaattgatagcttggatgagctctaacataacttctgaagagggttctgacggtgctgtgcagcccgatggcggtcaacccgctgttcgtaatgaacgtgctactggtggtggctctagtgatggtgctgttcagcctgacggtggtcaacctgctgtgcgcaacgagcgtgcaacaggaggtgaagaggaacaagtaagaacaatcatagtaaatcatgtcaatgatacctggggattaaaggtgagacaaagtttatggtttcatttgtcatgtttgacttttggacaacatacagttcaagaatttttagtaagttttggagtatggatcagaactccagctccatatagacctcctaatgcacccattctctccactcttcccgaacatacagtcattcaccaccatcatcaccactaa

SEQ ID NO: 50 - DNA, 717 bp, synthetic construct
  misc_feature 1..717: note = Canine parvovirus 3L17 peptide epitope in 4x configuration; monomer WhcAg
  source 1..717: mol_type = other DNA
atggacatagatccctataaagaatttggttcatcttatcagttgttgaattttctacctttggacttctttcctgacctaaatgctttggtggacactgctactgccttgtatgaagaagagctaacaggtcgagaacattgctctccgcaccatacagctattagacaagctttagtatgctgggatgaattaactaaattgatagcttggatgagctctaacataacttctgaagagggtgacggtgctgtgcagcccgatggcggtcaacccgctgttcgtaatgaacgtggtggctctgatggtgctgttcagcctgacggtggtcaacctgctgtgcgcaacgagcgtggtggttccgacggtgccgttcaacccgacggtggccaacccgccgtgcgtaatgagcgcggtggttctgacggcgctgtgcaacctgacggcggtcagcccgccgttcgtaacgagcgtggtgaagaggaacaagtaagaacaatcatagtaaatcatgtcaatgatacctggggattaaaggtgagacaaagtttatggtttcatttgtcatgtttgacttttggacaacatacagttcaagaatttttagtaagttttggagtatggatcagaactccagctccatatagacctcctaatgcacccattctctccactcttcccgaacatacagtcattcaccaccatcatcaccactaa

SEQ ID NO: 51 - DNA, 1152 bp, synthetic construct
  misc_feature 1..1152: note = IHNV vaccine; E1+E2 epitopes in tandem WHcAg dimer
  source 1..1152: mol_type = other DNA
atggacatagatccctataaagaatttggttcatcttatcagttgttgaattttctacctttggacttctttcctgacctaaatgctttggtggacactgctactgccttgtatgaagaagagctaacaggtcgagaacattgctctccgcaccatacagctattagacaagctttagtatgctgggatgaattaactaaattgatagcttggatgagctctaacataacttctgaacaagtaagaacaatcatagtaaatcatgtcaatgatacctggggattaaaggtgagacaaagtttatggtttcatttgtcatgtttgacttttggacaacatacagttcaagaatttttagtaagttttggagtatggatcagaactccagctccatatagacctcctaatgcacccattctctccactcttcccgaacatacagtcattggtggaagtggagggtctggtgggtccgggggtagtggtgggtctgatatcgatccctacaaagaattcggcagttcttatcagttactaaatttcctgccgctggatttttttcccgatctgaacgccttggtcgatactgccaccgccttgtacgaggaagagctaaccgggcgagagcattgtagtccacatcatactgctatccgccaggctctggtctgctgggacgaattgaccaagttaattgcatggatgagctccaatattactagtcctggtggaagtggagacgatgaaaatcgtggcttgatcgcttatcctaccagtatccgttccttgagtgtcggcggaagtggagggtctgatctgattagcgtggtttacaacagtggaagcgagatcctgtcgtttcctggtggatcaggggagcaggtccgcacgatcattgttaaccacgtcaacgatacctggggcctaaaggttcgccaatctttgtggttccatctgtcgtgcctgacctttgggcaacacaccgtccaggagttcctggtgagcttcggcgtttggatccgcaccccagcaccctaccgcccgccaaatgctcccattttaagtaccttgcccgaacacaccgtgattgagcaaaaattaattagcgaggaagacctagaacaaaaactgatctctgaagaggatctgtaa

SEQ ID NO: 52 - DNA, 1356 bp, synthetic construct
  misc_feature 1..1356: note = IHNV vaccine; DIII epitope in tandem WHcAg dimer
  source 1..1356: mol_type = other DNA
atggacatagatccctataaagaatttggttcatcttatcagttgttgaattttctacctttggacttctttcctgacctaaatgctttggtggacactgctactgccttgtatgaagaagagctaacaggtcgagaacattgctctccgcaccatacagctattagacaagctttagtatgctgggatgaattaactaaattgatagcttggatgagctctaacataacttctgaacaagtaagaacaatcatagtaaatcatgtcaatgatacctggggattaaaggtgagacaaagtttatggtttcatttgtcatgtttgacttttggacaacatacagttcaagaatttttagtaagttttggagtatggatcagaactccagctccatatagacctcctaatgcacccattctctccactcttcccgaacatacagtcattggtggaagtggagggtctggtgggtccgggggtagtggtgggtctgatatcgatccctacaaagaattcggcagttcttatcagttactaaatttcctgccgctggatttttttcccgatctgaacgccttggtcgatactgccaccgccttgtacgaggaagagctaaccgggcgagagcattgtagtccacatcatactgctatccgccaggctctggtctgctgggacgaattgaccaagttaattgcatggatgagctccaatattactagtcctggtggaagtggagacgatgagaacagggggctaattgcctatcccacatccatccggtccctgtcagtcggaaacgacggtggcagtggagggtctagccaagagataaaagctcacctctttgttgataaaatctccaatcgagtcgtgaaggcaacgagctacggacaccacccctggggactgcatcaggcctgtatgattgaattctgtgggcaacagtggatacggacagatctcggtgacctaatatctgtcgtatacaattctggatcagaaatcctctcgttcccgaagtgtgaagacaagaccgtgggaccagcagagggtggcggtccagcaggtggatcaggggagcaggtccgcacgatcattgttaaccacgtcaacgatacctggggcctaaaggttcgccaatctttgtggttccatctgtcgtgcctgacctttgggcaacacaccgtccaggagttcctggtgagcttcggcgtttggatccgcaccccagcaccctaccgcccgccaaatgctcccattttaagtaccttgcccgaacacaccgtgattgagcaaaaattaattagcgaggaagacctagaacaaaaactgatctctgaagaggatctgtaa

SEQ ID NO: 53 - protein, 58 aa, Plasmodium yoelii; identical to SEQ ID NO: 18

SEQ ID NO: 54 - protein, 15 aa, Plasmodium yoelii
NEDSYVPSAEQILEF

SEQ ID NO: 55 - DNA, 99 bp, Escherichia coli
  source 1..99: mol_type = other DNA
cgtatgaagcagatagaggacaagatcgaggaaatcctgtcgaaaatctatcacattgaaaatgagattgcacggataaagaaattgattggtgaaaga

SEQ ID NO: 56 - DNA, 99 bp, unidentified
  misc_feature 1..99: note = Arthrospira sp.
  source 1..99: mol_type = other DNA
cgcatgaaacaaattgaagataaaatcgaagagattctgagcaagatctatcacattgaaaacgagattgctcgtatcaaaaaattgatcggtgaacgt

SEQ ID NO: 57 - protein, 33 aa, unidentified
  REGION 1..33: note = 1GCM (GCN4-pII coiled-coil trimerization domain)
RMKQIEDKIEEILSKIYHIENEIARIKKLIGER

SEQ ID NO: 58 - DNA, 108 bp, Escherichia coli
  source 1..108: mol_type = other DNA
cgtttactgcagcggatcaagcagcaggaggataaactggaagagacactgtctaagatctaccatctggagaatgaaatagcaagagttaaaaaactgggagagcgc

SEQ ID NO: 59 - DNA, 108 bp, unidentified
  misc_feature 1..108: note = Arthrospira sp.
  source 1..108: mol_type = other DNA
cgtttgctgcaacgtattaagcaacaggaagataagttggaggaaactctgagcaagatctaccacttggaaaacgaaatcgctcgtgttaaaaaactgggcgagcgt

SEQ ID NO: 60 - protein, 36 aa, unidentified
  REGION 1..36: note = 1ce0 (GCN4-pII coiled-coil trimerization domain with mutations from HIV gp41)
RLLQRIKQQEDKLEETLSKIYHLENEIARVKKLGER

SEQ ID NO: 61 - DNA, 132 bp, Escherichia coli
  source 1..132: mol_type = other DNA
gtgtcggggcttgtacagcaacagaacaatatactacgcgcgttagaagcaacccagcatgccgtgcaggccctggtgtggggcgtaaagcagcttcaggccagagtattagcactggagcggtacattaag

SEQ ID NO: 62 - DNA, 132 bp, unidentified
  misc_feature 1..132: note = Arthrospira sp.
  source 1..132: mol_type = other DNA
gtgtccggcttggtgcaacaacaaaacaacatcctgcgtgctctggaagctacccaacacgctgttcaagccctggtgtggggtgttaaacaactgcaagctcgtgttttggctttggaacgctacattaag

SEQ ID NO: 63 - protein, 49 aa, unidentified
  REGION 1..49: note = 2zfc (N-terminal HIV gp41-derived)
QARQLVSGLVQQQNNILRALEATQHAVQALVWGVKQLQARVLALERYIK

SEQ ID NO: 64 - protein, 102 aa, synthetic construct
  REGION 1..102: note = IHNV DIII epitope
DDENRGLIAYPTSIRSLSVGNDGGSGGSSQEIKGHLFVDKISNRVVKATSYGHHPWGLHQACMIEFCGQQWIRTDLGDLISVVYNSGSEILSFPKCEDKTVG

SEQ ID NO: 65 - protein, 8 aa, synthetic construct
  REGION 1..8: note = HIV gp41 fusion peptide A epitope
AVGIGAVF

SEQ ID NO: 66 - protein, 8 aa, synthetic construct
  REGION 1..8: note = HIV gp41 fusion peptide B epitope
AVGLGAVF

SEQ ID NO: 67 - protein, 8 aa, synthetic construct
  REGION 1..8: note = HIV gp41 fusion peptide C epitope
AVGIGAMI

SEQ ID NO: 68 - protein, 508 aa, unidentified
  source 1..508: note = Infectious hematopoietic necrosis virus
MDTMITTPLILILITCGANSQTVKPDTASESDQPTWSNPLFTYPEGCTLDKLSKVNASQLRCPRIFDDENRGLIAYPTSIRSLSVGNDLGEIHTQGNHIHKVLYRTICSTGFFGGQTIEKALVEMKLSTKEAGAYDTTTAAALYFPAPRCQWYTDNVQNDLIFYYTTQKSVLRDPYTRDFLDSDFIGGKCTKSPCQTHWSNVVWMGDAGIPACDSSQEIKGHLFVDKISNRVVKATSYGHHPWGLHQACMIEFCGQQWIRTDLGDLISVVYNSGSEILSFPKCEDKTVGMRGNLDDFAYLDDLVKASESREECLEAHAEIISTNSVTPYLLSKFRSPHPGINDVYAMHKGSIYHGMCMTVAVDEVSKDRTTYRAHRATSFTKWERPFGDEWEGFHGLHGNNTTIIPDLEKYVAQYKMSMMEPMSIKSVPHPSILALYNETDVSGISIRKLDSFDLQSLHWSFWPTISALGGIPFVLLLAVAACCCWSGRPPTPSVPQSIPMYHLANRS

SEQ ID NO: 69 - protein, 584 aa, unidentified
  source 1..584: note = Canine parvovirus
MSDGAVQPDGGQPAVRNERATGSGNGSGGGGGGGSGGVGISTGTFNNQTEFKFLENGWVEITANSSRLVHLNMPESENYRRVVVNNLDKTAVNGNMALDDTHAQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNIYHGTDPDDVQFYTIENSVPVHLLRTGDEFATGTFFFDCKPCRLTHTWQTNRALGLPPFLNSLPQAEGGTNFGYIGVQQDKRRGVTQMGNTNIITEATIMRPAEVGYSAPYYSFEASTQGPFKTPIAAGRGGAQTDENQAADGDPRYAFGRQHGQKTTTTGETPERFTYIAHQDTGRYPEGDWIQNINFNLPVTNDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPSNIGGMKIVYEKSQLAPRKLY

SEQ ID NO: 70 - protein, 839 aa, Human immunodeficiency virus 1
MRVRGMQRNWQHLGKWGFLFLGILIICNAEDNLWVTVYYGVPVWKEATTTLFCASDAKGYEREVHNVWATHACVPTDPSPQEMVLENVTENFNMWKNEMVEQMHTDIISLWDQSLKPCVKLTPLCVTLNCTDVDTNRTQNDNMTEERGVLKNCSFNMTTEVKDKRLKVSALFYRLDVVPISNNSNSSEYRLINCNTSTIKQACPKVSWDPIPIHYCAPAGYAILQCRDKQFNGTGPCKNVSTVQCTHGIKPVVSTQLLLNGSLAEKDIIIRCQNISDNTKTIIVHLNESVQINCTRPNNNVVESIHLGPGQAFYATRRITGNIRKAYCNINGTQWNNTLERVKTKLKTYFNKTITFNSASGGDLEVTMHSFNCRGEFFYCNTSELFKNAITPNVTIILQCRIKQIINMWQGVGQAMYASPIAGSITCNSSITGLLLTRDGGNDNVSTEEIFRPGGGNMKDNWRSELYKYKVVKIEPLGVAPTRAKRQVVKRDKRAVGLGAVFLGFLGAAGSTMGAASITLMVQARQLLSGIVQQQNNLLRAIEAQQHMLQLTVWGIKQLQARLLAVERYLKDQQLLGIWGCSGKLICTTNVPWNSSWSNKSQEEIWGNMTWMEWEKEISNYSSTIYSLIEQSQNQQEKNEQELLALDKWTSLWNWFDISNWLWYIKIFIMIVGGLIGLRIVFAVLSIVNRVRKGYSPLSLQTLIPSPRGPDRPEGIEEGGGEQNRDRSVRLVNGFLALVWDDLRNLCLFSYRHLRDFILIAARTVDRGLRRGWEALKYLWNLIQYWSRELKNSTTSLLDTTAVVVAEGTDRVIEALQRAGRAVLNVPRRIRQGAERALL